(19) United States
(12) Patent Application Publication
(10) Pub. No.: US 2026/0055463 A1
(43) Pub. Date: Feb. 26, 2026

(54) Mapping CpG Sites to Quantify Aging Traits

(71) Applicant: The Brigham and Women's Hospital, Inc., Boston, MA (US)

(72) Inventors: Vadim N. Gladyshev, Brookline, MA (US); Kejun Ying, Boston, MA (US)

(21) Appl. No.: 19/104,861

(22) PCT Filed: Aug. 21, 2023
(86) PCT No.: PCT/US2023/030726

§ 371 Date: Feb. 19, 2025

Related U.S. Application Data
(60) Provisional application No. 63/371,877, filed on Aug. 19, 2022.

(51) Int. Cl. C12Q 1/6883; G16B 20/00; G16B 40/20; G16H 10/40; G16H 50/30

(52) U.S. Cl. CPC C12Q 1/6883; G16B 20/00; G16B 40/20; G16H 10/40; G16H 50/30; C12Q 2600/106; C12Q 2600/154

(57) ABSTRACT

Provided herein are methods that use methylation of causal CpG sites to quantify aging and predict whether an intervention will be protective or damaging to the aging process.

### CLAIM OF PRIORITY

This application claims the benefit of U.S. Provisional Application Ser. No. 63/371,877, filed on Aug. 19, 2022. The entire contents of the foregoing are incorporated herein by reference.

### FEDERALLY SPONSORED RESEARCH OR DEVELOPMENT

This invention was made with Government support under Grant No. AG065403 awarded by the National Institutes of Health. The Government has certain rights in the invention.

### TECHNICAL FIELD

Provided herein are methods that use methylation of causal CpG sites to quantify aging and predict whether an intervention will be protective or damaging to the aging process.

### BACKGROUND

Aging is a complex biological process characterized by a buildup of deleterious molecular changes that result in a gradual decline of function of various organs and systems and ultimately lead to death¹. Although the underlying mechanisms of aging are not well understood, various studies indicate that aging is strongly associated with changes in the epigenome, quantified as a set of chemical modifications to DNA and histones that affect gene expression and chromatin structure². DNA methylation is one of the best studied epigenetic modifications. In mammals, 5-methylcytosine (5mC) is the most common form of DNA methylation, which is achieved by the action of DNA methyltransferases (DNMTs)^{3,4}. Studies have shown that DNA methylation patterns change with age, wherein the global level of DNA methylation decreases slightly during adulthood, while some local areas may be hypomethylated or hypermethylated^{2,5-9}. Furthermore, the level of methylation of some specific CpG sites shows a strong correlation with age, which can be used to build machine learning-based models that can accurately predict the age of biological samples^{8,10}. As models can quantify age with very high accuracy, researchers termed these models epigenetic aging clocks (e.g., Horvath pan tissue epigenetic clock and Hannum blood based epigenetic clock)^{11,12}. The predicted age based on various epigenetic aging clocks appears to have a higher association with health-related measurements than chronological age^{13,14}. Therefore, it is believed that they could be used to better represent the biological age of samples than chronological age¹⁵.

### SUMMARY

Provided herein are methods comprising providing a biological test system, optionally a cell, tissue, organ, or organism; and determining methylation of one or more causal CpG sites identified in Tables A, B, and/or C. As used herein, determining can include performing an assay (or causing an assay to be performed) on a test system, or can include using existing methylation data.

In some embodiments, the methods include determining methylation of 5, 10, 20, 30, 40, 50, 60, 70, 80, 90, 100, 125, 150, or more of the causal CpG sites. In some embodiments, the methods include determining methylation of up to 100, 125, 150, 175, 200, 250, 300, 350, 400, 500, or 1,000 CpG sites, including determining methylation of at least 5, 10, 20, 30, 40, 50, 60, 70, 80, 90, 100, 125, 150, or all of the causal CpG sites.

In some embodiments, the methods also include applying an intervention to the system and determining methylation of the one or more causal CpG sites during and/or after an application of an intervention.

In some embodiments, the methods further include comparing the methylation of the one or more causal CpG sites to a reference pattern of methylation. In some embodiments, the methods include the reference pattern is a baseline methylation pattern obtained in the same test system before application of an intervention, or a level obtained earlier in time in the same test system, or a level or range in a reference system that represents a level or range of methylation in the absence of an intervention.

In some embodiments, the methods include determining methylation of a plurality of causal CpG sites, and calculating a score using an algorithm to calculate the cumulative effect on aging or aging-related outcomes, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; or neural networks.

In some embodiments, the methods include calculating a predicted age using the determined methylation and applying an algorithm to the levels.

In some embodiments, the algorithm comprises:

$$PredictedAge = intercept + b 1 * CpG 1 + b 2 * CpG 2 + … + bn * CpGn$$

Where b1−bn are the model coefficient ‘estimate’ from Tables A, B, and/or C and CpG1−CpGn are the methylation level of given CpG sites (on a scale of 0-1).

In some embodiments, the methods include identifying an intervention as having a protective effect when changes in methylation are observed that are consistent with protection, and/or identifying an intervention as having a damaging effect when changes in methylation are observed that are consistent with damage.

In some embodiments, the methods also include: selecting an intervention that has been identified as having a protective effect as a candidate intervention; applying the candidate intervention to an in vivo model of a disorder or condition associated with aging, optionally wherein the model is a non-human test animal or a human subject in a clinical trial; and determining whether the candidate intervention has a protective effect on the disorder or condition related to aging.

Also provided herein are methods of predicting an effect of an intervention on aging. The methods include: providing a biological test system, optionally a cell, tissue, organ, or organism; and determining methylation of one or more causal CpG sites identified in Tables A, B, and/or C; applying an intervention to the system, determining methylation of the one or more causal CpG sites during and/or after an application of an intervention; comparing the methylation of the one or more causal

CpG sites to a reference pattern of methylation; and identifying an intervention as having a protective effect on aging when changes in methylation are observed that are consistent with protection, and/or identifying an intervention as having a damaging effect on aging when changes in methylation are observed that are consistent with damage.

In some embodiments, the methods include determining methylation of 5, 10, 20, 30, 40, 50, 60, 70, 80, 90, 100, 125, 150, or more of the causal CpG sites.

In some embodiments, the methods include determining methylation of up to 100, 125, 150, 175, 200, 250, 300, 350, 400, 500, or 1,000 CpG sites, including determining methylation of at least 5, 10, 20, 30, 40, 50, 60, 70, 80, 90, 100, 125, 150, or all of the causal CpG sites.

In some embodiments, the reference pattern is a baseline methylation pattern obtained in the same test system before application of an intervention, or a level obtained earlier in time in the same test system, or a level in a reference system that represents the level of methylation in the absence of an intervention.

In some embodiments, the methods include determining methylation of a plurality of causal CpG sites, and calculating a score using an algorithm to calculate the cumulative effect on aging or age-related outcomes, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; or neural networks.

In some embodiments, the methods include calculating a predicted age using the determined methylation and applying an algorithm to the levels.

In some embodiments, the algorithm comprises:

$$PredictedAge = intercept + b 1 * CpG 1 + b 2 * CpG 2 + … + bn * CpGn$$

where b1−bn are the model coefficient ‘estimate’ from Tables A, B, and/or C and CpG1−CpGn are the methylation level of given CpG sites (on a scale of 0-1).

In some embodiments, the methods include selecting an intervention that has been identified as having a protective effect as a candidate intervention; applying the candidate intervention to an in vivo model of a disorder or condition associated with aging, optionally wherein the model is a non-human test animal or a human subject in a clinical trial; and determining whether the candidate intervention has a protective effect on the disorder or condition related to aging.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Epigenome-wide Mendelian Randomization on various aging-related phenotypes. a. Schematic diagram shows the principle of MR using meQTLs as exposures and aging-related traits as outcomes to identify putative causal CpG sites. b. Flow chart shows the procedure for epigenome-wide MR and sensitivity analysis. c. Number of significant putative causal CpG sites identified for each trait after adjusting for multiple tests using the Bonferroni correction. Red regions of the bars indicate the number of putative causal CpG sites supported by the colocalization analysis with conditional PP-H4>0.7. d. Spearman correlation of the estimated causal effects of CpGs in twelve traits. Only CpGs with significant MR signals across at least six traits are included in the analysis. Color scheme reflects Spearman correlation coefficients, * adjusted P<0.05, ** adjusted P<0.01, *** adjusted P<0.001. e. Modified Mississippi plot shows significant MR signals for Aging-GIP1. X-axis corresponds to the genomic positions of CpG sites; Y-axis represents the size of the causal effect adjusted by colocalization probability (PP-H4). CpG sites with top adjusted causal effects are annotated with the name and nearest gene. Only CpG sites with adjusted P<0.05 are included in the plot.

FIGS. 2A-F. CpG sites causal to aging are enriched in specific genetic regulatory regions. a. Bar plot shows enrichment of putative causal CpG sites in 14 Roadmap genomic annotations. Y axis shows-log 10 (FDR) based on Fisher's exact test, signed by log 2(Odds ratio). Putative causal CpG sites identified for different traits are annotated with different colors. Two dotted horizontal lines show the FDR threshold of 0.05. TssA, active transcription start site. Prom, upstream/downstream TSS promoter. Tx, actively transcribed state. TxWk, weak transcription. TxEn, transcribed and regulatory Prom/Enh. EnhA, active enhancer. EnhW, weak enhancer. DNase, primary DNase. ZNF/Rpts, state associated with zinc finger protein genes. Het, constitutive heterochromatin. PromP, Poised promoter. PromBiv, bivalent regulatory states. ReprPC, repressed polycomb states. Quies, quiescent state. b, c. Box plot shows distribution of conservation scores in causal and non-putative causal CpG sites for Aging-GIP1. Conservation scores are obtained by Learning Evidence of Conservation from Integrated Functional genomic annotations (LECIF, b) and phastCons (c). * P<0.05, ** P<0.01, *** P<0.001, **** P<0.0001. d. Enrichment of putative causal CpG sites for 12 aging-related traits against transcription-factor-binding sites. Each horizontal bar represents an enriched term. The X-axis shows the −log 10 (P-value), signed by log 2 (Odds ratio). The top 10 enriched terms that passed the FDR threshold of 0.05 for each direction are annotated. e. Scatter plot showing the mediation analysis of Aging-GIP1. The total causal effects are shown on X-axis and the direct effects of DNA methylation are shown on Y-axis. The color shows the proportion of DNA methylation (DNAm) causal effect that is mediated by gene expression. Top CpG-gene pairs are annotated. f. Enrichment of the top mediator gene for Aging-GIP1 in GO terms (above dashed line) and KEGG pathways (below the dashed line). The X-axis shows −log(P) for the fisher exact test.

FIGS. 3A-G. MR on epigenetic age successfully recovers clock sites as putative causal CpG sites. a. For epigenetic age measurements, true causal sites are the clock sites and the sites upstream of clock sites. We used these traits as a positive control to validate the MR approach. b. Forest plot shows enrichment of clock sites for each model in putative causal CpG sites. For each clock trait, putative CpG sites are identified by MR using corresponding clock traits as outcome. X-axis shows the log 2(Odds Ratio). P-values calculated by Fisher's exact test are annotated. Error bars show 95% confidence intervals. Different colors represent different thresholds for putative causal CpGs. c-e. Correlation between ground truth causal effects (clock coefficients, X-axis) and causal effects estimated by MR (Y-axis, using GWAS of corresponding clocks as outcome traits) for Hannum age (c), Horvath age (d) and PhenoAge (e). Different colors represent different thresholds for putative causal CpGs. Pearson's correlation coefficients and P-values are annotated. f. Receiver operating characteristic (ROC) curves show sensitivity (Y-axis) and 1-specificity (X-axis) of MR in identifying putative causal CpG sites for clock traits, with the area under the ROC curve (AUC) annotated. g. Forest plot shows enrichment of clock sites for six aging clock models in putative causal CpG sites identified by MR for each trait. X-axis shows the log 2(Odds Ratio). P-values calculated by Fisher's exact test are annotated if P<0.05. Error bars show 95% confidence intervals. Different colors represent the different thresholds for putative causal CpGs.

FIGS. 4A-C. Integration of causal information and age-associated differential methylation to separate protective and damaging epigenetic changes. a. Schematic diagram showing the method to identify protective and damaging epigenetic changes by integrating MR results and age-related differential methylation. b. Relationship between MR-estimated causal effects (X-axis) and age-related differential methylation (Y-axis) for each significant putative causal CpG identified in Aging-GIP1. The color scheme highlights the expected impact of age-related differential methylation on aging. Error bars show the standard error of b. The size reflects the PP-H4. Only CpG sites with adjusted P-values<0.05 and relative PP-H4>0.7 are plotted. The CpG sites with the top 10 largest effect sizes are annotated. c. Area plots show the total cumulative effect of changes in DNA methylation on Aging-GIP1. X-axis shows the rank of top 3,000 CpG sites based on the magnitude of age-associated differential methylation. Y-axis and the color scheme show the P-value estimated by 10,000 permutation tests.

FIGS. 5A-E. Construction and application of causality-informed epigenetic clocks. a. Schematic diagram shows the procedure of constructing causality-informed epigenetic clocks. b. Scatter plots show the accuracy of causal clocks on the test set. The X-axis shows the real age of each sample, and the Y-axis shows the predicted age of the same sample based on each clock model. Median absolute error (MAE) and Pearson's R are annotated. c. Line plot showing the relationship between causality factor (t) and clock accuracy measured by MAE and Pearson's R. d. Line plot shows the relationship between the causality factor (τ) and −log 10 (p) for the association with mortality risk (signed by log 2(hazard ratio)) estimated from the meta-analysis of FHS and WHI cohorts. Yellow dashed line shows the P threshold of 0.05. Hazard ratio of mortality risk for every 10-year increase in age for each clock model and the 95% confidential interval for τ=0.3 is annotated. Results based on Horvath age, Hannum age, and PhenoAge are also shown by arrows for comparison. e. Scatter plots show the application of causal clocks and five other aging clocks to reprogramming of fibroblasts to iPSCs. X-axis shows days after initiating reprogramming. Pearson's R and P values are annotated.

FIGS. 6A-C. Causality-informed epigenetic clocks can better capture aging-related effects. a. Box plots show the association between epigenetic age and aging-related conditions, including atherosclerosis, prostate cancer prognosis, and hypertensive heart disease. b. Box plots show the association between epigenetic age and damaging conditions, including smoking, Progeroid Syndromes, and Sun exposure. Scatter plots show the correlation between epigenetic age and blood PON1 activity. Epigenetic age prediction is rescaled to a 0-1 scale for better comparison. The color scheme shows the PON1 genotype in subjects. Linear regression is performed, and Pearson's R and P values are annotated. c. Box plots show the association between epigenetic age and short-term treatments, including the umbilical cord blood plasma treatment, 15 months of cigarette smoke condensate (CSC) treatment, and 6-week supplementation of overweight subjects with omega-3 fatty acids. For umbilical cord blood plasma treatment, paired sign-test was performed, and the color scheme and the pie chart indicate whether the subject is rejuvenated after treatment based on the corresponding clock. For unpaired box plots, significant pairs based on two-tail t-test are annotated with stars. * P<0.05, ** P<0.01, *** P<0.001, **** P<0.0001.

FIG. 7. Genetic correlation between 12 lifespan-related phenotypes. Genetic correlations were calculated using LDSC. Areas of the squares represent absolute values of corresponding genetic correlations. Genetic correlations that could not be estimated are shown as blanks. P values are corrected using Bonferroni correction for the number of tests, * P nominal <0.05, ** P adjusted <0.05, *** P adjusted <0.01.

FIGS. 8A-B. Relationship between meSNPs and causal CpGs. Forest plot shows enrichment of meSNP among causal CpGs. Error bar shows the 95% confidential interval. P-value of significant results is annotated (8A). Scatter plot shows Pearson's correlation between the effect of a single CpG site estimated by MR and a single meSNP (8B). Correlation coefficient and P-value are annotated at the top.

FIG. 9. Relationship between estimated causal effects and evolutionary conservation. Box plot shows the distribution of conservation scores in causal and non-causal CpG sites. Conservation scores were obtained by Learning Evidence of Conservation from Integrated Functional genomic annotations (LECIF), phastCons, and phyloP. * P<0.05, ** P<0.01, *** P<0.001, **** P<0.0001.

FIG. 10. Enrichment analysis. Bar plot shows enrichment of causal CpG sites in genomic annotations. Y-axis shows-log 10 (FDR) based on Fisher's exact test, signed by log 2(Odds Ratio). Causal CpG sites identified for different traits are annotated with different colors. Two dotted horizontal lines show the FDR threshold of 0.05.

FIG. 11. Enrichment of causal CpG sites among CpG sites that show age-related changes. Error bar indicates the 95% confidence interval. Bar plot shows the signed-log 10 (P-value) of Spearman's correlation between age-related change and causal effect size. The orange dotted line shows the threshold of P<0.05.

### DETAILED DESCRIPTION

Although epigenetic aging clocks provide a useful tool for profiling biological aging, they should be used with caution, as they are built based on pure correlations 16. It is unclear whether differential DNA methylation used to predict age is causal to aging-related phenotypes or simply represents byproducts of the aging process that do not influence aging themselves. To establish a causal relationship, the gold standard approach is the application of randomized controlled trials (RCT), where participants are randomly assigned to the intervention arm that receives the treatment or the control arm. As the randomization step balances all confounding factors between two arms, the differences observed in the outcome between two groups are purely driven by the intervention; thus, the causal effect can be estimated¹⁷. However, given the large number of CpG sites across the genome, it is inefficient and infeasible to perform the perturbation on each of them and assess the aging-related outcomes.

Mendelian randomization (MR) is a genetic approach to causal inference that recapitulates the principle of RCT. Instead of perturbing an exposure through treatment, the MR uses the genetic variants that are robustly associated with the exposure as instrumental variables^{18,19}. As genetic variants of parental DNA are naturally randomly passed on to the offspring, the effect estimated by MR is not affected by environmental confounders and thus can be considered as an estimation of a causal effect, similar to the RCTs. In recent years, several studies have shown that MR can be applied to molecular traits by using the genetic variants associated with molecular levels as instruments (also known as molecular quantitative trait loci, molQTL)²⁰. These molecular QTLs include gene expression (eQTL)²¹, RNA splicing (sQTL)²², plasma protein (pQTL)²³, metabolites (mQTL)²⁴, as well as DNA methylation (meQTL)²⁵. A previous study showed that it is feasible to use meQTLs as instruments to identify putative causal CpG sites for diseases²⁶. By integrating molQTLs with genome-wide association studies for traits such as lifespan, healthspan, extreme longevity, and other measurements related to aging, it is biologically plausible to perform two-sample MR to estimate the causal effects of molecular changes on the aging process.

Here, we leveraged large-scale genetic data and performed epigenome-wide Mendelian Randomization (EWMR) on 420,509 CpG sites to identify CpG sites that are causal to twelve aging-related traits. We found that none of the existing clocks are enriched for putative causal CpG sites. We further constructed a causality-informed clock based on this inferred causal knowledge, as well as clocks that separately measure damaging and protective changes. Their applications provide direct insights into the aging process. Thus, our results offer a comprehensive map of human CpG sites causal to aging traits, which can be used to build causal biomarkers of aging and assess novel anti-aging interventions and aging-accelerating events.

Many existing epigenetic aging clock models accurately predict the age of samples⁸, and there are numerous CpG sites that are differentially methylated during aging²⁷. DNA methylation levels affect the structure of chromatin and the expression of neighboring genes^{28,29}, through which they can causally affect aging-related phenotypes. A recent study also suggested that DNA methylation may play a causal role in the rejuvenation effect observed during iPSC reprogramming³⁰. However, it is important to understand whether age-related differential DNA methylation causes aging-related phenotypes and which of its components do it. A previous transcriptome-wide MR study revealed that differentially expressed genes in human diseases mainly reflect gene expression caused by disease rather than disease-causing genes³¹. Similarly, differential DNA methylations during aging may primarily reflect the downstream effects of aging phenotypes rather than causing them. Our EWMR findings support this notion as we found no significant overlap between CpG sites causal to healthy longevity and those differentially methylated during aging.

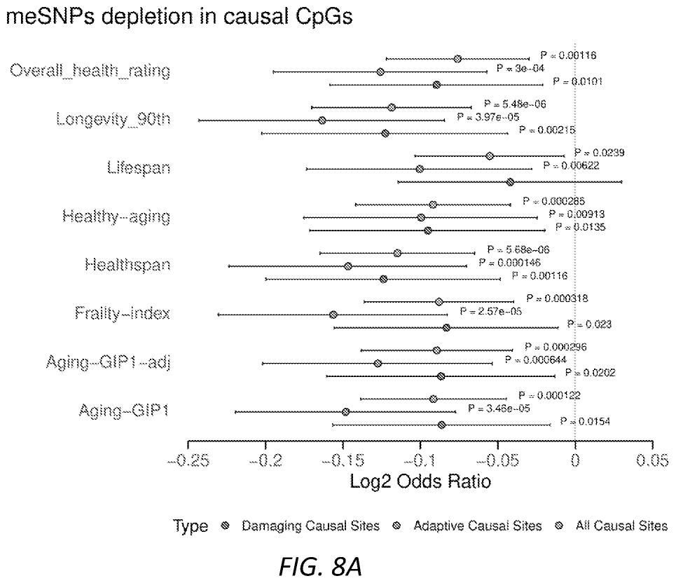

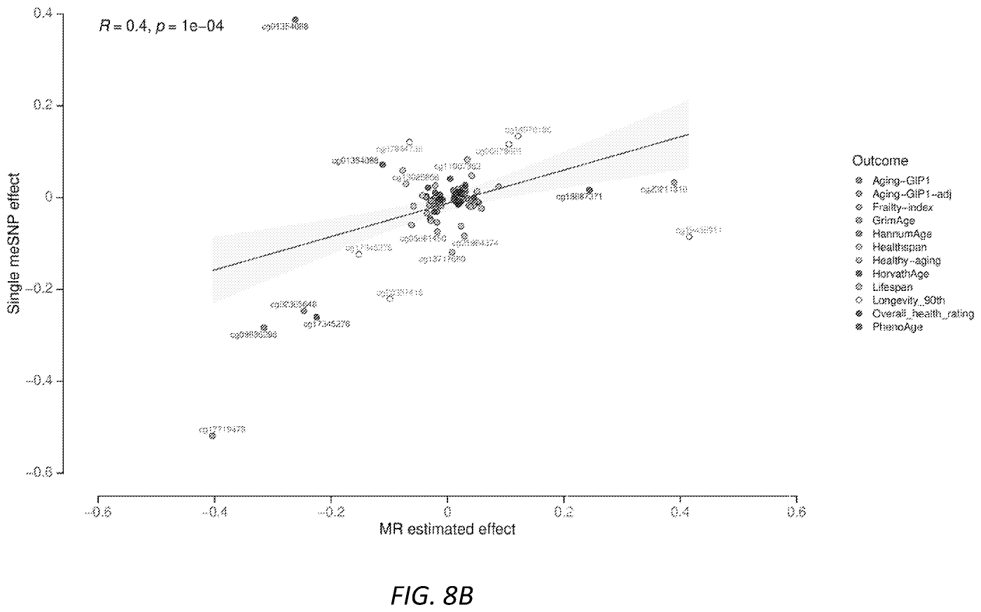

MR is a powerful method to identify causal relationships between exposure traits and phenotypes³². However, it is limited by the availability of genetic instruments for the exposure traits. In our study, we utilized the DNA meQTLs of 420,509 CpG sites from the Illumina 450K methylation array as instrumental variables to infer their causal relationship with aging-related phenotypes. However, there are many unmeasured CpG sites across the genome, and the methylation patterns of nearby CpG sites are highly correlated²⁸. Therefore, it is not possible to fully separate the causal effect of a single CpG and its neighbors. Analysis of point mutations at putative causal CpG sites (meSNPs) suggests that the epimutation of a single causal CpG site identified by MR may be sufficient to alter the phenotype (FIGS. 8A-B). However, due to the lack of abundance of meSNPs on putative causal CpG sites, this hypothesis is difficult to test across all causal CpG sites identified. Therefore, we tend to reach a more conservative conclusion and think that the putative causal CpG sites identified in our study serve as tagging CpG sites for causal regulatory regions in aging-related phenotypes. Future genome-wide meQTL studies may facilitate further analyses of causal effects of CpG sites at base-pair resolution.

The genetic instruments of CpG sites for our study were selected from the currently largest meQTL study in whole blood (GoDMC, 36 cohorts, including 27,750 European subjects). Therefore, the CpG sites we identified are valid in blood. However, a previous study showed that up to 73% cis-meQTLs are shared across tissues (including blood, brain, and saliva)³³. This suggests that the identified putative causal CpG sites also act in other tissues to affect lifespan and healthspan.

We found that TF-binding sites of BRD4 and CREB1 are enriched with CpG sites whose methylation levels promote healthy longevity, and TF-binding sites for HDAC1 are enriched with CpG sites whose methylation levels decrease healthy longevity. BRD4 is known to contribute to cell senescence and promote inflammation³⁴. Therefore, our findings suggest that higher DNA methylation at BRI)+binding sites may inhibit the downstream effects of BRD4 and promote healthy longevity. Similarly, previous studies showed that CREB1 is related to type II diabetes and neurodegeneration 35 and mediates the effect of calorie restriction 36. However, how DNA methylation may affect CREB1 binding is not well understood. Our data suggest that higher methylation at CREB1-binding sites may support its longevity effects. HDAC1 is a histone deacetylase, and its activity increases with aging and may promote age-related phenotypes^{30,37}. HDAC1 has been shown to specifically bind to methylated sites. Our data, therefore, support the hypothesis that HDAC1 plays a damaging role during aging, as increased DNA methylation at HDAC1 binding sites may causally inhibit healthy longevity.

One general approach for developing anti-aging interventions is to identify molecular changes during aging and use these changes as targets to modulate the aging process^{38,39}. A similar idea has also been applied to evaluate potential longevity interventions. However, this logic is intrinsically flawed, as correlation does not imply causation and age-associated differential methylation are not necessarily causal to age-associated declines. As living organisms are complex systems with various adaptive mechanisms, many molecular changes during aging are potentially neutral downstream effects of fundamental damaging changes or even adaptive mechanisms that protect against aging phenotypes. This notion is usually underappreciated as age-associated differential methylation are assumed to be damaging. As a result, adaptive mechanisms of aging are largely understudied. However, there is evidence to suggest that at least some age-associated differential methylation is protective against aging phenotypes.

An example of age-related protective changes is the Insulin and IGF-1 signaling (IIS) pathway. Attenuation of IIS signaling intensity through multiple genetic manipulations has been shown to consistently extend the lifespan of worms, flies, mice, and potentially humans^{40,41}. This pathway also mediates pro-longevity effects of dietary restriction⁴⁰. Growth hormone is produced by the anterior pituitary gland and can induce the production of IGF-1, thus increasing IIS signaling. Both growth hormone and IGF-1 levels decline during aging⁴², which is considered to be a defensive response that extends lifespan⁷. Another example of an age-related adaptation is protein aggregation. It has been shown in C. elegans that the protein aggregation events are increased during aging. Although it may look like a result of losing proteostasis, it turns out to be a protective mechanism that drives aberrant proteins into insoluble aggregates to improve overall proteostasis, and has been observed in long-lived mutants⁴³. Similar protective mechanisms are also observed in mouse nerves at the transcriptomic level⁴⁴.

The present results suggest that adaptive mechanisms at the epigenetic level are nearly as common as damaging changes and that simply following age-associated differential methylation in DNA methylation does not allow us to infer positive, neutral, or negative effects on age-related traits. However, the identified damaging and protective CpG sites are extremely useful both for understanding aging and quantifying it, and the same applies to rejuvenation. Together, the identified CpGs represent causal epigenetic changes, and their combined effect on health-related phenotypes is negative.

The framework we described for epigenetic changes in this study may be applied to any other age-related change, e.g., changes in the transcriptome, metabolome, and proteome. While all age-related features may be used to construct aging clocks, some of them are expected to be negative, some neutral, and some protective. Neither the direction nor the degree of age-associated differential methylation is important, and inferring the need to bring these changes to those observed in the young state as a way to rejuvenate an organism is equally incorrect. Instead, the focus should be on the causal effects of age-associated differential methylation, as well as on the direction of their effect. The present causal analysis was conducted using blood samples because large meQTL studies are only available in blood up-to-date⁴⁵. However, previous studies suggest that the cis-meQTLs are conserved across tissues³³, therefore the present findings are also likely applicable to other tissues.

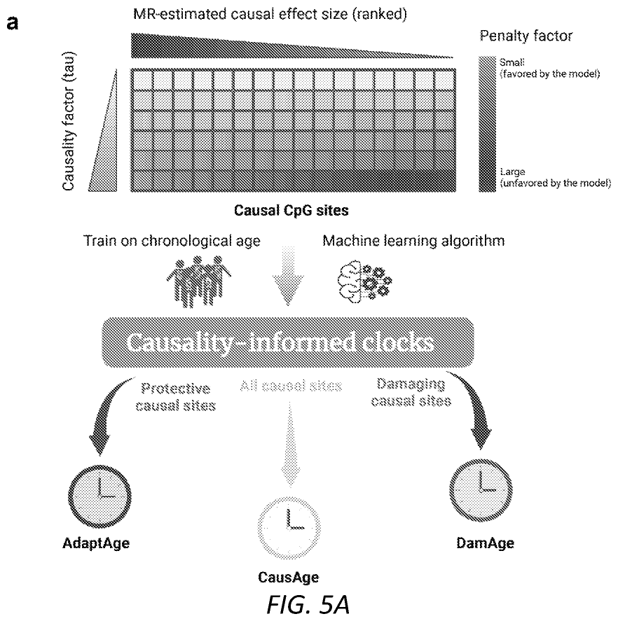

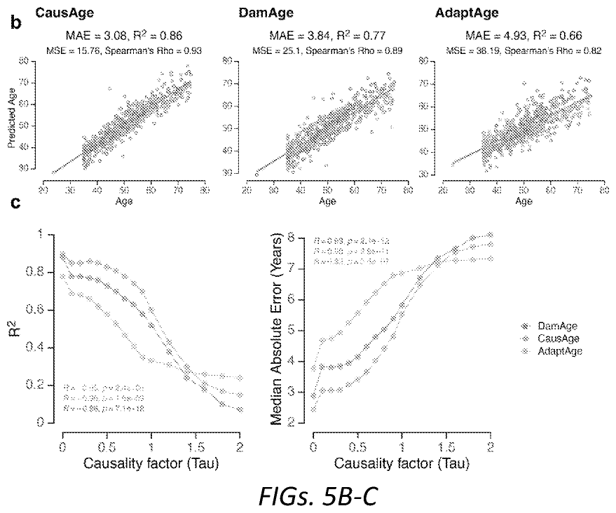

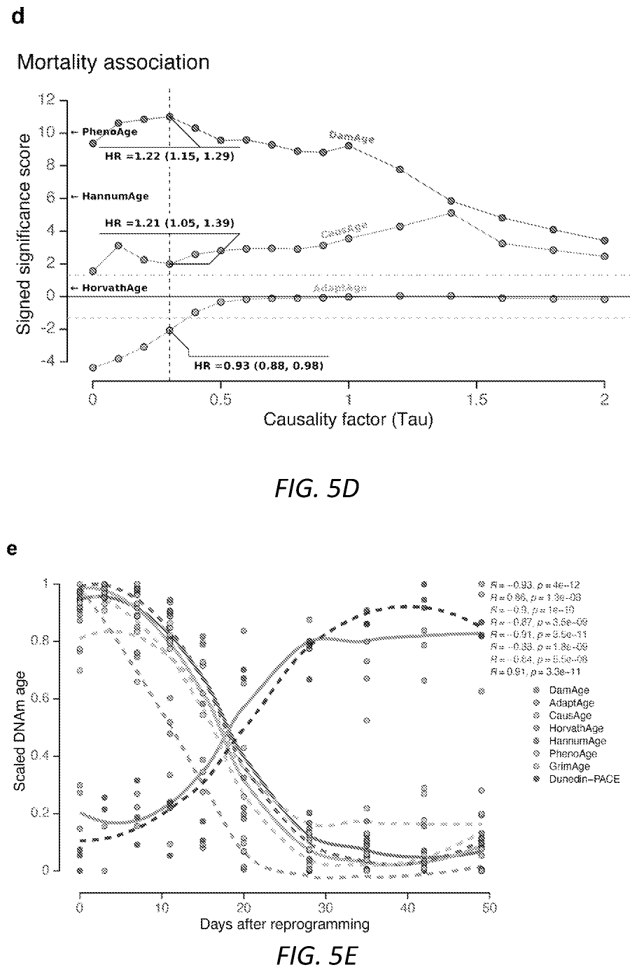

The causal epigenetic clock models, CausAge, AdaptAge, and DamAge, could help separate protective changes from damaging events. We also showed that by preselecting the CpG sites that show protective adaptation during aging, it is possible to build an aging clock showing an inverse relationship with mortality. Specifically, subjects with elevated protective adaptation are predicted to be age-accelerated by AdaptAge and have a lower risk of mortality (FIG. 5c). Similarly, AdaptAge shows an inverse relationship with rejuvenation (e.g., iPSC reprogramming) and aging acceleration. Note that both DamAge and AdaptAge show similar accuracy in predicting chronological age, but their delta-age term reflects an opposite biological meaning. Although we observed a weak positive correlation between DamAge and AdaptAge in the general population, this correlation may be due to collider bias and survival bias^{46,47}, e.g., both DamAge and AdaptAge contribute to mortality and the individuals with high DamAge and low AdaptAge are removed from the population due to higher mortality risk, thus resulting in an apparent positive correlation. The causality-informed clock models described herein provide novel insights into the mechanisms of aging and provide methods for testing interventions to delay aging and reverse biological age.

### Test Systems

Thus, provided herein are methods for identifying compounds or conditions that can be used to monitor effects of various interventions on methylation of CpG sites that affect aging, and to identify interventions that can delay or reverse the aging process in a tissue or a subject.

The methods can be practiced using a biological test system, including one or more human cells, all or part of a human tissue, or all or part of an human organ. The cell can be, e.g., a mammalian cell, such as a primary cell (including erythrocytes; platelets; peripheral blood mononuclear cells (PBMC), e.g., lymphocytes, monocytes, or macrophages; bone marrow cells; endothelial cells, e.g., vascular or bronchial endothelial cells; pancreatic islet beta cells; renal cells; hepatocytes; neurons and glia; epidermal cells; respiratory interstitial cells; adipocytes; dermal fibroblasts; muscle cells; cells of the eye (e.g., photoreceptors, RPE cells, retinal ganglia cells) or ear (e.g., hair cells or supporting cells); or hair follicles. Primary or cultured cells including stem cells and immortalized cells can also be used, e.g., induced pluripotent stem cells (iPSCs), embryonic stem cells (ES cells), hematopoietic stem cells (HSCs), mesenchymal stem cells (MSCs), pre-adipocytes, and neural progenitor cells. Cultured cells such as HEK293 and fibroblasts can also be used.

The tissues can be, e.g., connective tissue, epithelial tissue, muscle tissue, and nervous tissue. The organs can be, e.g., capillaries; joints; nerves; skin; tendons; arteries; cerebellum; liver; nasal cavity; spleen; tongue; appendix; diaphragm; lungs; ovaries; scrotum; thyroid; adrenal glands; ears; larynx; esophagus; stomach; trachea; brain; eyes; ligaments; penis; spinal cord; thymus gland; bones; fallopian tubes; lymph nodes; pancreas; small intestine; ureters; bronchi; genitals; large intestine; pharynx; salivary glands; urethra; bladder; gallbladder; lymphatic vessel; placenta; skeletal muscles; uterus; bone marrow; heart; mouth; prostate; seminal vesicles; vulva; bulbourethral glands; hair follicle; mesentery; pineal gland; subcutaneous tissue; veins; colon; hypothalamus; mammary glands; pituitary gland; teeth; vagina; cervix; interstitium; nose; parathyroid glands; tonsils; vas deferens; clitoris; kidneys; nails; anus; rectum; or testes.

In some embodiments, the biological test system is whole blood, or a cell from an embryo, e.g., a human embryo.

In some embodiments, a whole organism is used; the organism can be, e.g., a human, optionally a human subject in a clinical trial or a veterinary subject in a clinical trial, or a non-human model animal, e.g., a non-human mammal such as a mouse, rat, or rabbit, or can be a nematode, insect (e.g., drosophila), yeast, or bacterium.

### Interventions

The present methods can include applying one or more interventions to the test system. Interventions can include, for example, administration of one or more compounds, e.g., polypeptides, polynucleotides, or inorganic or organic large or small molecule test compounds. The intervention can also be, e.g., alteration of an environmental factor, e.g., food (e.g., quality or quantity of nutrition, calories, or type); exposure to toxic or potentially toxic environments (e.g., to mimic exposure to pollution or smoking); oxygen levels; and so on. When more than one intervention is applied, the more than one can include multiple applications over time of the same intervention, or application of multiple interventions, e.g., at the same time or consecutively or over time.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. Natural compounds such as vitamins and neutraceuticals can also be tested using the present methods.

### Methods for Determining Effects on Aging

The present methods include determining methylation of one or more causal CpG sites identified herein, i.e., in Tables A, B, and/or C. In some embodiments, the methods include determining methylation of 5, 10, 20, 30, 40, 50, 60, 70, 80, 90, 100, 125, 150, or more causal CpG sites described herein; in some embodiments, the methods include determining methylation of up to 100, 125, 150, 175, 200, 250, 300, 350, 400, 500, or 1,000 CpGs, including at least 5, 10, 20, 30, 40, 50, 60, 70, 80, 90, 100, 125, 150, or all of the causal CpG sites described herein. The methods can include applying an intervention to the system and determining methylation of the one or more CpG sites during and/or after application of the intervention.

As used herein, determining can include performing an assay (or causing an assay to be performed) on a test system, or can include using existing methylation data. Methods (assays) for determining methylation of a specific site are known in the art, and include sodium bisulfite conversion and sequencing (e.g., next-generation sequencing (NGS)), differential enzymatic cleavage of DNA, CpG DNA methyltransferase, and affinity capture of methylated DNA; DNA affinity capture methods include methylated DNA immunoprecipitation (Me-DIP) that uses a methyl DNA specific antibody, or methyl capture using methyl-CpG binding domain (MBD) proteins. See, e.g., Tang et al., Methods Mol Biol. 2015; 1238:653-75; Chatterjee et al., Methods Mol Biol. 2017; 1537:249-277; Beck, Nat Biotechnol. 2010 October; 28(10):1026-8; Nair et al., Epigenetics. 2011 January; 6(1):34-44; Hsu et al., Methods Mol Biol. 2020; 2102:225-234; Feng and Lou, Methods Mol Biol. 2019; 1894:181-227.

In some embodiments, the methods include comparing methylation of one or more causal CpG sites identified herein to a reference pattern of methylation. The reference pattern can be, e.g., a baseline obtained in the same test system, or a level or range obtained earlier in time in the same test system, or a level or range in a reference system that represents the level or range of methylation in the absence of an intervention. The reference system is typically the same type as the test system (i.e., a matched control) and be as identical to the test system as possible.

A test compound can be identified as having a protective effect when changes in methylation are observed that are consistent with protection as shown herein, i.e., reduce the age predicted by DamAge; conversely, a test compound can be identified as having a damaging effect when changes in methylation are observed that are consistent with damage as shown herein, i.e., increase the age predicted by DamAge. A change in methylation associated with a damaging effect will have the same directionality as shown in Table B, and a change in methylation associated with a protective effect will have the same directionality as shown in Table C. Where a plurality (more than one) level of methylation is determined, an algorithm can be used to calculate the cumulative effect on aging, e.g., manual or software-based modeling algorithms such as a linear algorithms, e.g., a rank-based linear algorithm; principal component analysis (PCA); classification or decision trees; elastic net analysis; linear and polynomial support vector machines (SMV); shrunken centroids; random forest algorithms; support vector machines; or neural networks.

For example, the methods can include calculating a predicted age using the determined levels of methylation and applying an algorithm to the levels. An exemplary algorithm is as follows:

$$PredictedAge = intercept + b 1 * CpG 1 + b 2 * CpG 2 + … + bn * CpGn$$

Where b1−bn are the model coefficient ‘estimate’ from Table A and CpG1−CpGn are the methylation level of given CpG sites (on a scale of 0-1, e.g., 0.7 means 70% methylated).

A similar algorithm can be used to quantify the age-related damage effect or protective effect of interventions using the model from tables B and C, respectively.

In some embodiments, the methods include summing the product of methylation difference and causal effect estimate for each cPG site, and determining if the sum is positive (i.e., more adaptation, thus protective) or negative (i.e., more damage, thus damaging). In some embodiments, the difference of the predicted age before and after treatment is used. For example, DamAge measures age-related damage, and if it is increased means that there are damage accumulated (usually bad). AdaptAge measure age-related adaptation/protection, if it is increased means that the adaptation is increased (usually good but can be bad or neutral)

An intervention that has been screened by a method described herein and determined to have a protective effect on aging can be considered a candidate compound. A candidate compound that has been screened, e.g., in an in vivo model of a disorder such as a non-human test animal or a human subject in a clinical trial, and determined to have a protective effect and/or a desirable effect on aging, e.g., on one or more symptoms of aging, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

The methods can also be used to identify interventions that are damaging, e.g., that can speed aging or cause premature aging; such interventions can be identified for avoidance or exclusion, e.g., in food, cosmetics, or pharmaceuticals.

Test compounds identified as protective hits can be considered candidate therapeutic compounds, useful in slowing, delaying, or even reversing aging. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test interventions identified as candidate protective interventions compounds can be further screened by administration to a test system in an animal model of aging, e.g., as described herein. The animal can be monitored for a change in aging, e.g., for an improvement in a parameter of aging, e.g., a parameter related to health or clinical outcome. In some embodiments, the parameter is development of age-related conditions such as hearing loss, cataracts and refractive errors, back and neck pain and osteoarthritis, chronic obstructive pulmonary disease, diabetes, and dementia, and an improvement would be a delay or decrease in risk of development of one or more age-related conditions. In some embodiments, the test system is epidermis, and the parameter is development of age-related skin conditions such as thinning, sagging, wrinkling, xerosis, pruritis, eczematic dermatitis, purpura, and chronic venous insufficiency, and an improvement would be a delay or decrease in risk of development of one or more age-related conditions.

TABLE A
CausAge CpG sites and estimates

| term | estimate |
|---|---|
| (Intercept) | 86.8081638 |
| cg00027162 | 1.66785269 |
| cg00048759 | 5.41958522 |
| cg00200653 | −0.26977 |
| cg00347863 | 4.10387211 |
| cg00505045 | 12.0066436 |
| cg00563845 | −0.54509 |
| cg00603274 | 0.1468829 |
| cg00614360 | 1.188062 |
| cg00655552 | −0.9658713 |
| cg00663739 | 3.54837844 |
| cg00715290 | −10.219189 |
| cg00879155 | 0.61301692 |
| cg00910168 | −1.1934856 |
| cg00962755 | 1.0630155 |
| cg01035616 | 1.81673898 |
| cg01048752 | −1.1464102 |
| cg01105058 | 5.10947032 |
| cg01274524 | 0.29682433 |
| cg01321673 | 0.84088792 |
| cg01329511 | 3.69864743 |
| cg01334432 | −1.8983001 |
| cg01399860 | −0.3860917 |
| cg01421252 | −0.8981966 |
| cg01454752 | 5.88142509 |
| cg01503516 | 1.83103817 |
| cg01538166 | −4.8332151 |
| cg01557754 | −4.61235 |
| cg01579218 | −2.4021833 |
| cg01597480 | −2.8061322 |
| cg01762785 | 0.2491522 |
| cg01791648 | −5.1331644 |
| cg01835620 | −4.7227796 |
| cg01902704 | 0.30891281 |
| cg01971089 | −5.7406146 |
| cg01988129 | −0.2660887 |
| cg02059055 | 6.14037906 |
| cg02088403 | −0.3011643 |
| cg02153490 | −0.0921455 |
| cg02161761 | 2.86628373 |
| cg02204442 | −1.3815779 |
| cg02225085 | 2.25807193 |
| cg02232751 | 4.2091458 |
| cg02254885 | −14.127701 |
| cg02256105 | −0.020307 |
| cg02306162 | −0.0499936 |
| cg02339392 | −0.8155854 |
| cg02361878 | 6.17892437 |
| cg02462416 | 1.28635049 |
| cg02462487 | −2.7352981 |
| cg02493740 | −1.6079316 |
| cg02501978 | −1.5130389 |
| cg02722637 | 0.46587786 |
| cg02729030 | −5.2153005 |
| cg02763536 | 0.61200038 |
| cg02767634 | 1.97373793 |
| cg02867102 | −10.428584 |
| cg02870946 | −1.2303554 |
| cg02942825 | −2.7979306 |
| cg02965178 | 2.52384557 |
| cg03046819 | −7.3084773 |
| cg03092551 | −0.1670602 |
| cg03155027 | −0.0080934 |
| cg03164928 | −3.5957803 |
| cg03167948 | −0.1844817 |
| cg03203114 | −1.2488165 |
| cg03227963 | −0.0943263 |
| cg03277049 | 5.124818 |
| cg03283486 | 0.20334065 |
| cg03438101 | 2.0009373 |
| cg03446427 | −0.3279279 |
| cg03520471 | −1.506224 |
| cg03552151 | 2.4012138 |
| cg03573179 | −2.7746358 |
| cg03588998 | −0.4807417 |
| cg03604424 | 5.43833432 |
| cg03664992 | 31.8703656 |
| cg03823084 | −3.1743293 |
| cg03834467 | −0.9860636 |
| cg03839949 | −10.265735 |
| cg03844971 | −1.5663285 |
| cg03848890 | −1.9066197 |
| cg03869874 | 4.16917666 |
| cg03883502 | 10.5911797 |
| cg03887528 | −0.3733555 |
| cg03950166 | −1.558685 |
| cg03982897 | 0.4693096 |
| cg03986400 | −1.2139964 |
| cg04088674 | −0.7946084 |
| cg04129308 | 1.85528241 |
| cg04154465 | 3.06782338 |
| cg04157658 | −1.7570169 |
| cg04229059 | −6.4451535 |
| cg04267526 | 0.73174734 |
| cg04270358 | 1.03213167 |
| cg04338863 | −3.3058175 |
| cg04407388 | −1.6827815 |
| cg04445851 | 0.04240196 |
| cg04451175 | −1.2048828 |
| cg04508114 | −0.4335248 |
| cg04512892 | 1.47577894 |
| cg04531704 | 0.91127887 |
| cg04673465 | 2.22081179 |
| cg04742397 | −4.6831153 |
| cg04753583 | 0.41593537 |
| cg04760708 | 0.70927954 |
| cg04785213 | 0.70161645 |
| cg04786857 | 0.17092297 |
| cg04838627 | −1.1294458 |
| cg04911050 | 4.68390918 |
| cg04998671 | −30.960837 |
| cg05001334 | 1.03380228 |
| cg05003422 | 5.60587721 |
| cg05034363 | 2.92504981 |
| cg05059607 | 2.60469202 |
| cg05070268 | −1.2215682 |
| cg05087948 | 1.1991173 |
| cg05090759 | −1.397405 |
| cg05172940 | 2.42024622 |
| cg05238695 | −3.3973555 |
| cg05260372 | −1.7723352 |
| cg05265042 | −0.2320468 |
| cg05280698 | 4.0511649 |
| cg05310309 | 1.55600268 |
| cg05360774 | 1.25613217 |
| cg05376617 | 0.55323002 |
| cg05395210 | −4.1241958 |
| cg05455729 | −0.3270445 |
| cg05463027 | −14.37923 |
| cg05470939 | 2.07754589 |
| cg05561193 | −1.3461417 |
| cg05726118 | 2.53588419 |
| cg05861879 | −4.0260888 |
| cg05874888 | −0.8083869 |
| cg05900234 | 4.74763611 |
| cg05922911 | 0.43707176 |
| cg05966235 | 0.13642299 |
| cg05980111 | −1.7713828 |
| cg05991454 | 7.601516 |
| cg06007201 | −12.449463 |
| cg06024411 | 1.52945724 |
| cg06089468 | 3.46444719 |
| cg06156376 | 4.91061933 |
| cg06179486 | −1.1384817 |
| cg06275642 | 0.8324924 |
| cg06449934 | −0.175352 |
| cg06470822 | 0.70317313 |
| cg06493612 | 0.59124635 |
| cg06574296 | −0.0119487 |
| cg06594770 | 4.51135658 |
| cg06639733 | −4.3949057 |
| cg06658468 | −2.1231767 |
| cg06670463 | −4.5280541 |
| cg06672696 | 10.5030019 |
| cg06675483 | −0.9069373 |
| cg06713116 | −0.135586 |
| cg06734510 | −8.9034084 |
| cg06739520 | 5.13696838 |
| cg06799422 | 5.01718809 |
| cg06851000 | 0.93928715 |
| cg06882058 | 3.6706885 |
| cg06885782 | −2.5846568 |
| cg06916725 | 2.37227624 |
| cg06933824 | 3.45347204 |
| cg06980387 | 5.82627257 |
| cg06984176 | 0.14159407 |
| cg07155455 | 1.56473154 |
| cg07155684 | 2.89603282 |
| cg07186576 | −0.3358436 |
| cg07286682 | 0.68887153 |
| cg07360805 | −0.773523 |
| cg07390013 | 0.81041383 |
| cg07495704 | −7.6333551 |
| cg07495811 | 2.41235283 |
| cg07560510 | 2.49582803 |
| cg07657357 | 3.3756577 |
| cg07671586 | 1.07640333 |
| cg07725123 | 1.05559048 |
| cg07736657 | −0.0639274 |
| cg07809027 | −3.4112001 |
| cg07833467 | −0.8148068 |
| cg07850154 | −24.234776 |
| cg07910813 | −1.0729767 |
| cg07984980 | −1.1511983 |
| cg08017858 | 0.82022065 |
| cg08025960 | 0.29773201 |
| cg08046569 | 1.11084516 |
| cg08081725 | −0.9547729 |
| cg08108311 | 8.88841119 |
| cg08122369 | −10.534933 |
| cg08129490 | 1.09065667 |
| cg08166232 | −0.428682 |
| cg08170837 | 2.04109329 |
| cg08173606 | 1.06622431 |
| cg08190615 | 2.1362206 |
| cg08274097 | 2.64302111 |
| cg08301612 | −2.729191 |
| cg08317738 | 0.26269708 |
| cg08332662 | −0.9323952 |
| cg08402963 | 0.65459904 |
| cg08415508 | −0.6785189 |
| cg08462924 | 2.21313438 |
| cg08529529 | −8.9566255 |
| cg08627089 | 0.12873143 |
| cg08637514 | 2.15114206 |
| cg08671671 | 1.33511191 |
| cg08688335 | −3.7960263 |
| cg08733522 | −0.2023862 |
| cg08762484 | 4.64062708 |
| cg08797606 | 14.2895947 |
| cg08826281 | −2.6358023 |
| cg08841511 | 2.65823287 |
| cg08863440 | −1.9901855 |
| cg08916461 | 0.8140773 |
| cg08931376 | −1.0839649 |
| cg08965235 | −1.7659968 |
| cg09012544 | 11.0253743 |
| cg09063262 | −0.2829372 |
| cg09164168 | 0.45031532 |
| cg09185587 | −0.4486915 |
| cg09278098 | −0.8205643 |
| cg09279566 | −1.0027833 |
| cg09361966 | −0.9050816 |
| cg09415366 | 1.02554887 |
| cg09450197 | 1.77830844 |
| cg09550397 | −5.8053873 |
| cg09573389 | 1.64146009 |
| cg09607276 | 0.94574236 |
| cg09662798 | −2.7371179 |
| cg09896106 | 0.84267082 |
| cg09906309 | 1.65079676 |
| cg09937438 | −2.2196985 |
| cg09974041 | 6.62067597 |
| cg10046620 | 4.54479927 |
| cg10078511 | −0.0416903 |
| cg10110474 | 0.80717511 |
| cg10243676 | −1.6251679 |
| cg10245988 | 1.55349551 |
| cg10253371 | 3.96217418 |
| cg10406027 | −3.2117192 |
| cg10421002 | 1.3635941 |
| cg10489614 | −0.2639964 |
| cg10515671 | −6.0152328 |
| cg10529555 | −3.6603521 |
| cg10547057 | −1.1200554 |
| cg10557683 | 5.05242507 |
| cg10577534 | −5.5688057 |
| cg10616300 | 3.87926028 |
| cg10619644 | −0.8571093 |
| cg10693071 | 0.47751499 |
| cg10695490 | −3.8320014 |
| cg10715265 | 0.21336365 |
| cg10750934 | −1.9992943 |
| cg10755878 | −0.0094494 |
| cg10809491 | 4.26757702 |
| cg10923036 | 0.77527103 |
| cg10951117 | −0.8091901 |
| cg10958002 | −0.2601732 |
| cg10960709 | 3.34339293 |
| cg10975001 | 5.13128448 |
| cg10999479 | −0.0700323 |
| cg11053663 | 0.51629203 |
| cg11180122 | 0.38160951 |
| cg11229399 | 5.00155235 |
| cg11244402 | 2.8480487 |
| cg11326793 | −4.2479654 |
| cg11369071 | −0.4989324 |
| cg11524642 | −0.3645636 |
| cg11545887 | −0.3978629 |
| cg11573608 | −0.5950011 |
| cg11792186 | −2.8525112 |
| cg11835347 | −6.0889257 |
| cg11846333 | −6.070178 |
| cg11946583 | −1.2843143 |
| cg11954355 | −1.3845087 |
| cg11960655 | −0.0902339 |
| cg12003463 | −4.6515317 |
| cg12007048 | −3.949833 |
| cg12023170 | −2.7268671 |
| cg12027899 | 1.58172957 |
| cg12042659 | −1.3789434 |
| cg12148898 | −0.384638 |
| cg12172441 | 2.25939625 |
| cg12179288 | 9.03260201 |
| cg12211856 | 5.53003549 |
| cg12212060 | −1.8165233 |
| cg12226009 | −0.2716606 |
| cg12257692 | 1.93368443 |
| cg12283398 | 1.16841617 |
| cg12316010 | −2.4446777 |
| cg12387865 | 0.08803323 |
| cg12414301 | 1.42398185 |
| cg12419195 | 9.72263643 |
| cg12419685 | 6.52999266 |
| cg12419863 | −1.7071248 |
| cg12614395 | 1.49723017 |
| cg12666263 | 5.00932125 |
| cg12788037 | 4.95628137 |
| cg12833018 | 1.64883965 |
| cg12908607 | −0.4231708 |
| cg12978308 | 1.6373962 |
| cg13001893 | 3.04127886 |
| cg13098855 | 0.4802255 |
| cg13202122 | 5.57624045 |
| cg13224583 | 4.62226563 |
| cg13258563 | −1.1869121 |
| cg13444538 | 0.06770674 |
| cg13483882 | −1.7886747 |
| cg13485809 | 12.169601 |
| cg13511324 | 0.46275927 |
| cg13561879 | 3.62771253 |
| cg13569146 | 4.98657616 |
| cg13665684 | −3.7799892 |
| cg13690424 | 23.2094324 |
| cg13721134 | 3.62308515 |
| cg13798745 | −0.0001335 |
| cg13813086 | −0.1890568 |
| cg13817265 | −4.0834617 |
| cg13826452 | 2.5553349 |
| cg13956645 | 3.95818583 |
| cg13983063 | −0.0171946 |
| cg14018471 | −0.7749422 |
| cg14067761 | 0.19897087 |
| cg14095101 | −1.4001818 |
| cg14241323 | 0.94905355 |
| cg14593290 | 3.14809644 |
| cg14611152 | 1.32505591 |
| cg14634687 | 4.47111967 |
| cg14672293 | 13.5739468 |
| cg14765414 | 4.14891241 |
| cg14848077 | −0.767323 |
| cg14989252 | −0.2316609 |
| cg15031579 | 2.70728813 |
| cg15038286 | −1.0398195 |
| cg15046909 | 0.200946 |
| cg15086884 | 5.14813184 |
| cg15156071 | 4.58189083 |
| cg15205507 | 0.23859528 |
| cg15213491 | −0.9306521 |
| cg15241130 | −0.0182477 |
| cg15270892 | 8.25017133 |
| cg15299997 | −5.1127891 |
| cg15383520 | 0.29117217 |
| cg15443907 | −0.969291 |
| cg15481172 | 0.61712916 |
| cg15596301 | 0.36470394 |
| cg15605172 | 1.6791391 |
| cg15622917 | 3.05433735 |
| cg15751090 | 1.97192522 |
| cg15787227 | −2.7295258 |
| cg15863539 | 2.20910781 |
| cg15964523 | 0.6993149 |
| cg16004055 | 4.4570457 |
| cg16008966 | −15.807545 |
| cg16080876 | 2.4362956 |
| cg16098332 | 0.15184917 |
| cg16193278 | −15.411232 |
| cg16195091 | 2.59835513 |
| cg16209444 | −11.317501 |
| cg16248756 | 2.36742112 |
| cg16312002 | 4.63900786 |
| cg16321524 | −4.5736367 |
| cg16427513 | −2.0005936 |
| cg16511841 | 5.28105424 |
| cg16562257 | 2.85337053 |
| cg16591681 | −3.9963812 |
| cg16633951 | 0.33813012 |
| cg16636110 | −0.7183975 |
| cg16701167 | 0.23260948 |
| cg16762979 | −10.463298 |
| cg16810279 | −4.4907529 |
| cg16886581 | 0.30612081 |
| cg16888547 | −11.387985 |
| cg16983588 | −2.1515222 |
| cg17092956 | 1.45618529 |
| cg17263013 | 0.18858046 |
| cg17272642 | −2.4585006 |
| cg17274064 | −1.3867088 |
| cg17298973 | 1.94992 |
| cg17304222 | 1.90388058 |
| cg17319774 | 0.99698296 |
| cg17344932 | −5.5540327 |
| cg17373751 | −1.6554786 |
| cg17390562 | −8.6232068 |
| cg17436666 | 2.18928834 |
| cg17459635 | 3.68913438 |
| cg17494199 | −0.1715263 |
| cg17514226 | −5.6434186 |
| cg17516572 | −0.4198843 |
| cg17526103 | 2.03529389 |
| cg17545662 | −0.5688546 |
| cg17576375 | −9.294081 |
| cg17646721 | 2.84486456 |
| cg17658733 | −4.7640179 |
| cg17664577 | −0.5925515 |
| cg17681698 | 1.2490976 |
| cg17745234 | −1.8125513 |
| cg17848389 | 2.38731434 |
| cg17956485 | −1.7032024 |
| cg17968880 | 2.24821429 |
| cg18050997 | 4.41984339 |
| cg18070470 | 2.53529387 |
| cg18137414 | −0.7303666 |
| cg18180155 | 0.81790443 |
| cg18196295 | −0.5853404 |
| cg18320379 | −0.2215413 |
| cg18327056 | 9.46785717 |
| cg18365211 | −0.0793333 |
| cg18468088 | 27.4876419 |
| cg18538662 | 2.79988528 |
| cg18644286 | −0.9799952 |
| cg18735810 | 0.68244159 |
| cg18775149 | 8.32237881 |
| cg18797590 | −5.8194081 |
| cg18958126 | 3.02207694 |
| cg19039841 | −0.8405036 |
| cg19043574 | 3.33108161 |
| cg19065831 | 0.27184155 |
| cg19120897 | −3.6694208 |
| cg19247841 | 14.723453 |
| cg19261426 | −6.075937 |
| cg19285688 | 4.08541936 |
| cg19399220 | 0.93454395 |
| cg19475108 | 0.06192032 |
| cg19511338 | −0.5194758 |
| cg19570154 | 0.41303613 |
| cg19692192 | −2.571877 |
| cg19812283 | 5.98507316 |
| cg19935065 | 3.82916957 |
| cg19953038 | 3.98511867 |
| cg19955500 | −13.876876 |
| cg20059012 | −2.1235345 |
| cg20141652 | 1.30401269 |
| cg20147046 | 4.1143005 |
| cg20235117 | −2.0613692 |
| cg20245568 | 0.68005345 |
| cg20320656 | 0.38267004 |
| cg20326410 | 11.201295 |
| cg20368283 | 5.35696824 |
| cg20494635 | −3.9640405 |
| cg20532887 | 0.60259149 |
| cg20666917 | 2.18261687 |
| cg20704028 | 2.79610397 |
| cg20711218 | 3.41249842 |
| cg20780880 | 1.00148751 |
| cg20816447 | −2.6993394 |
| cg20856545 | −0.4101868 |
| cg20861237 | −0.279039 |
| cg20957370 | 3.95246859 |
| cg21004924 | 0.70819353 |
| cg21121119 | −1.1258615 |
| cg21154793 | −1.3037331 |
| cg21160290 | 0.38957443 |
| cg21236593 | −0.0652929 |
| cg21249152 | −1.5108204 |
| cg21293242 | 8.88572867 |
| cg21329085 | 3.77841251 |
| cg21492308 | 1.9473482 |
| cg21527708 | −0.0998074 |
| cg21571060 | −3.4786234 |
| cg21635854 | 5.12401889 |
| cg21642251 | −2.4883914 |
| cg21697134 | −1.7557309 |
| cg21737698 | 2.0027006 |
| cg21796167 | 0.3492162 |
| cg21904251 | 3.55752964 |
| cg22013564 | −7.5597036 |
| cg22040809 | 8.66741839 |
| cg22189725 | 7.14460451 |
| cg22202381 | 2.51265264 |
| cg22215631 | 3.379229 |
| cg22225219 | 1.13297438 |
| cg22271663 | 0.87765391 |
| cg22277154 | −4.8170056 |
| cg22284745 | −0.6854184 |
| cg22442168 | 0.44928633 |
| cg22652782 | −1.6209717 |
| cg22681495 | 2.3134632 |
| cg22697325 | 5.05111063 |
| cg22698998 | −0.9411975 |
| cg22737282 | 0.86816672 |
| cg22761482 | 0.87556058 |
| cg22807700 | 2.15317892 |
| cg22872478 | 2.50465484 |
| cg22887526 | −0.2401105 |
| cg22889918 | 1.08548241 |
| cg22942200 | −2.9175601 |
| cg23065100 | −0.6004525 |
| cg23067299 | −2.1841151 |
| cg23112821 | −3.6469509 |
| cg23124451 | −10.036495 |
| cg23210521 | 1.44651388 |
| cg23260993 | 3.89894021 |
| cg23266598 | −6.8488037 |
| cg23280730 | −3.4125631 |
| cg23282585 | −1.7229973 |
| cg23285059 | 0.7204405 |
| cg23361092 | 10.3822605 |
| cg23542533 | 1.026108 |
| cg23600866 | 2.0216572 |
| cg23626546 | −8.707351 |
| cg23634477 | 1.27451964 |
| cg23690166 | −0.5875285 |
| cg23698023 | −12.985634 |
| cg23736055 | −1.357795 |
| cg23788418 | 1.27724752 |
| cg24010402 | −0.2930937 |
| cg24158141 | 1.04632039 |
| cg24171453 | 2.75547998 |
| cg24479590 | 5.84523578 |
| cg24670151 | 0.32400661 |
| cg24690437 | 4.35128473 |
| cg24710309 | 0.30843471 |
| cg24741744 | 2.91739227 |
| cg24760922 | −8.080979 |
| cg24768116 | 0.42211861 |
| cg24784350 | 3.84589631 |
| cg24870774 | 8.22822408 |
| cg24891133 | 4.96581286 |
| cg24921858 | −1.2505857 |
| cg24929896 | −0.2920107 |
| cg24934400 | 4.43299139 |
| cg24949488 | 0.25582433 |
| cg24952754 | −1.9056489 |
| cg24977886 | 1.26836239 |
| cg24987259 | −6.9437478 |
| cg25151919 | 0.95746929 |
| cg25152404 | −0.2924506 |
| cg25326896 | 25.0987055 |
| cg25339052 | −3.6695421 |
| cg25365379 | −0.5510868 |
| cg25399541 | 1.341181 |
| cg25473981 | −10.150787 |
| cg25519723 | 1.11150865 |
| cg25645064 | 7.2631839 |
| cg25667997 | −1.3546743 |
| cg25732028 | −0.8587237 |
| cg25770948 | 0.9165665 |
| cg25809722 | −10.614293 |
| cg25830305 | 0.47634254 |
| cg25893857 | −0.4712614 |
| cg25932066 | −0.4194662 |
| cg25945090 | 2.04267414 |
| cg25956966 | −1.7359694 |
| cg25961618 | 0.9160339 |
| cg25979108 | −0.7123194 |
| cg26025543 | −5.5626749 |
| cg26070099 | 1.69812005 |
| cg26084258 | −9.3631758 |
| cg26168651 | 0.82825267 |
| cg26235243 | −1.6907373 |
| cg26364871 | −1.6094513 |
| cg26365925 | 0.27623319 |
| cg26467949 | −2.4875227 |
| cg26635214 | −6.3784403 |
| cg26636010 | −1.3822866 |
| cg26780581 | 6.21876127 |
| cg26795848 | −0.3618613 |
| cg26808167 | 0.75740509 |
| cg26863750 | −1.6048529 |
| cg26888530 | 32.19979 |
| cg26935333 | −1.1980731 |
| cg26936171 | 5.42365829 |
| cg27021512 | −9.0782511 |
| cg27045062 | −7.4391165 |
| cg27051315 | −6.2630039 |
| cg27096232 | −1.1273398 |
| cg27175491 | −0.9469938 |
| cg27300045 | −4.5334548 |
| cg27321750 | 4.60763475 |
| cg27346545 | −5.6040311 |
| cg27355006 | −3.4021639 |
| cg27379915 | −0.8737653 |
| cg27391693 | −3.0055387 |
| cg27436995 | 2.16001599 |
| cg27489373 | 1.18664998 |
| cg27516159 | −2.4480191 |
| cg27529647 | −2.6392064 |
| cg27567593 | −5.8765869 |
| cg27587195 | 7.68513477 |
| cg27631597 | −0.435063 |
| cg27646965 | −9.0142653 |
| ch.17.1184801R | 0.12060794 |
| ch.2.75889792R | −1.5595759 |
| ch.4.73355803R | 1.41903713 |
| ch.8.353716R | 3.65921441 |

TABLE B
DamAge CpG Sites and Estimates

| term | estimate |
|---|---|
| (Intercept) | 543.431589 |
| cg00003994 | 0.11111111 |
| cg00023464 | 0.14209005 |
| cg00049440 | 0.8157786 |
| cg00052482 | −4.8587539 |
| cg00073543 | 0.47707559 |
| cg00084338 | 0.50020653 |
| cg00115654 | 0.39611731 |
| cg00117599 | 0.98554316 |
| cg00192773 | 0.2023957 |
| cg00228017 | 0.35894259 |
| cg00296038 | 0.05865345 |
| cg00300637 | −1.7170812 |
| cg00310410 | 0.60140438 |
| cg00330279 | −2.1801709 |
| cg00332802 | 0.95662949 |
| cg00346985 | 0.4613796 |
| cg00423487 | 0.87030153 |
| cg00462168 | 0.16728625 |
| cg00488692 | 0.06815366 |
| cg00512563 | 0.15401732 |
| cg00523379 | −3.0033172 |
| cg00534318 | 0.02684841 |
| cg00554993 | −7.1082145 |
| cg00563845 | −5.6154139 |
| cg00603274 | 0.0425444 |
| cg00612299 | 0.48533664 |
| cg00614360 | 0.15448162 |
| cg00645579 | 0.45600991 |
| cg00655552 | −4.90756 |
| cg00697033 | 0.88599752 |
| cg00717825 | −2.1476926 |
| cg00720845 | 0.61214374 |
| cg00757033 | 0.44314717 |
| cg00773060 | 0.34820322 |
| cg00788025 | −0.1715341 |
| cg00800780 | 0.22015696 |
| cg00864474 | 0.15943825 |
| cg00877212 | −3.0675184 |
| cg00894378 | −0.7248034 |
| cg00911370 | 0.42585708 |
| cg00998451 | 0.34737712 |
| cg01005582 | −2.6003224 |
| cg01023759 | 0.32995896 |
| cg01032119 | 0.35233375 |
| cg01077274 | 0.54770756 |
| cg01082242 | −18.039429 |
| cg01091514 | 0.49710385 |
| cg01103827 | −1.0552951 |
| cg01109734 | −10.38002 |
| cg01136167 | −0.3005004 |
| cg01146808 | −3.7485225 |
| cg01161889 | 0.99876084 |
| cg01168235 | 0.25733168 |
| cg01205087 | 0.85419248 |
| cg01220680 | 0.16150351 |
| cg01221209 | 0.57827344 |
| cg01302136 | 0.98595622 |
| cg01321673 | 0.27591904 |
| cg01329511 | 0.85832301 |
| cg01439105 | 0.06443618 |
| cg01449677 | −2.9864245 |
| cg01454752 | 0.11978521 |
| cg01461718 | 0.04750103 |
| cg01486146 | 0.41470467 |
| cg01518465 | 0.44816192 |
| cg01524149 | 0.22883106 |
| cg01557547 | 0.22222222 |
| cg01557754 | −14.19144 |
| cg01577414 | 0.40685667 |
| cg01597480 | −13.20968 |
| cg01603290 | 0.03148933 |
| cg01615339 | 0.08178439 |
| cg01619129 | 0.94671623 |
| cg01659184 | 0.92234614 |
| cg01682285 | 0.44609665 |
| cg01687862 | 0.02395704 |
| cg01691194 | −0.7867722 |
| cg01711322 | 0.370095 |
| cg01770362 | 0.16563404 |
| cg01791648 | −11.290423 |
| cg01831904 | −0.3972666 |
| cg01835620 | −10.874175 |
| cg01896085 | −7.7666605 |
| cg01900832 | −2.7591091 |
| cg01902704 | 0.98760843 |
| cg01905210 | 0.07476249 |
| cg01927000 | −2.0163627 |
| cg01964856 | 0.32869366 |
| cg02012043 | 0.16551544 |
| cg02021288 | 0.40726972 |
| cg02042310 | 0.23089632 |
| cg02058357 | 0.98802148 |
| cg02059055 | 0.43659645 |
| cg02088403 | −8.860954 |
| cg02171545 | −0.6243651 |
| cg02192678 | −0.8125605 |
| cg02208820 | 0.9748038 |
| cg02218884 | 0.38289963 |
| cg02230495 | −7.3797251 |
| cg02232751 | 0.55679471 |
| cg02244288 | 0.57954852 |
| cg02247160 | 0.86864932 |
| cg02254551 | 0.78314746 |
| cg02254885 | −31.562052 |
| cg02256455 | 0.33126807 |
| cg02283691 | −3.6355907 |
| cg02339392 | −13.600875 |
| cg02414626 | −9.5996382 |
| cg02462416 | 0.78149525 |
| cg02462487 | −10.990453 |
| cg02464608 | −0.8675449 |
| cg02466947 | 0.00206526 |
| cg02492920 | 0.08508881 |
| cg02578470 | 0.24246179 |
| cg02593958 | 0.49442379 |
| cg02595575 | 0.10408922 |
| cg02598071 | 0.41140025 |
| cg02610222 | 0.01486989 |
| cg02693210 | 0.1928955 |
| cg02697373 | 0.45022718 |
| cg02704502 | 0.70879802 |
| cg02762115 | 0.45146634 |
| cg02771117 | 0.47748864 |
| cg02773041 | 0.33209418 |
| cg02794779 | 0.53655514 |
| cg02822381 | 0.40437836 |
| cg02825527 | −1.5373353 |
| cg02867102 | −22.53154 |
| cg02897366 | 0.24659232 |
| cg02952809 | 0.42709624 |
| cg02955354 | −3.9663651 |
| cg02965712 | 0.4031392 |
| cg02975922 | −1.647494 |
| cg03000848 | 0.36735029 |
| cg03021329 | 0.37092111 |
| cg03036592 | −5.8444584 |
| cg03058664 | −0.1974869 |
| cg03071793 | 0.60057827 |
| cg03092551 | −8.0041195 |
| cg03162143 | −6.4974051 |
| cg03167948 | −9.4438113 |
| cg03214087 | −6.2794582 |
| cg03231447 | −3.4719549 |
| cg03283486 | 0.91449814 |
| cg03286774 | −1.0264384 |
| cg03303325 | 0.35398596 |
| cg03336167 | 0.42213961 |
| cg03338903 | 0.88806278 |
| cg03360992 | 0.28831062 |
| cg03508235 | 0.92523751 |
| cg03519011 | 0.60305659 |
| cg03520471 | −13.57242 |
| cg03555424 | −0.4071458 |
| cg03565475 | 0.99834779 |
| cg03593550 | 0.22180917 |
| cg03598731 | 0.57992565 |
| cg03622371 | −1.1468497 |
| cg03694580 | 0.89880215 |
| cg03719092 | 0.51631557 |
| cg03723356 | 0.90747625 |
| cg03732007 | 0.59396943 |
| cg03734594 | 0.33415944 |
| cg03777083 | 0.88310615 |
| cg03778594 | −1.7054577 |
| cg03780701 | 0.53531599 |
| cg03783925 | 0.61090458 |
| cg03805684 | 0.99958695 |
| cg03817794 | 0.68938455 |
| cg03843656 | −0.6039877 |
| cg03848483 | 0.23502685 |
| cg03857047 | −4.9085639 |
| cg03869874 | 0.19537381 |
| cg03882270 | 0.99421727 |
| cg03895593 | 0.54399009 |
| cg03923640 | 0.22717885 |
| cg03926598 | −2.3249287 |
| cg03950166 | −14.369518 |
| cg03950599 | 0.10491532 |
| cg03980370 | 0.68484097 |
| cg03987653 | −1.4263816 |
| cg03990139 | 0.12616306 |
| cg04000281 | 0.50185874 |
| cg04030848 | 0.42461793 |
| cg04038163 | 0.63403552 |
| cg04042468 | 0.45642297 |
| cg04091063 | 0.51094589 |
| cg04120413 | 0.25361421 |
| cg04214075 | 0.07889302 |
| cg04218812 | 0.21065675 |
| cg04218880 | 0.21106981 |
| cg04229059 | −19.174025 |
| cg04231636 | 0.14869888 |
| cg04307987 | −1.8838152 |
| cg04322572 | −0.3511388 |
| cg04336659 | 0.48451053 |
| cg04348250 | 0.12639405 |
| cg04358463 | 0.30152829 |
| cg04367197 | 0.11152416 |
| cg04399631 | 0.31557208 |
| cg04407388 | −6.3839574 |
| cg04418999 | 0.99297811 |
| cg04445851 | 0.36761669 |
| cg04474049 | −1.42296 |
| cg04505252 | 0.67410161 |
| cg04528771 | −5.803852 |
| cg04571584 | 0.99669558 |
| cg04655136 | −0.2616534 |
| cg04658021 | 0.82817018 |
| cg04666465 | 0.35811648 |
| cg04673465 | 0.41222635 |
| cg04691795 | −3.9202809 |
| cg04694619 | 0.73667277 |
| cg04717802 | −0.327324 |
| cg04751549 | 0.54688145 |
| cg04753583 | 0.50103263 |
| cg04781580 | 0.54812061 |
| cg04784327 | 0.7228418 |
| cg04785284 | −6.4082037 |
| cg04788957 | 0.87360595 |
| cg04820362 | −0.0635938 |
| cg04845466 | 0.25857084 |
| cg04872689 | 0.54729451 |
| cg04956585 | −1.4331823 |
| cg04995300 | 0.49153242 |
| cg05034363 | 0.01817431 |
| cg05045517 | −1.2198021 |
| cg05048976 | 0.34613796 |
| cg05055782 | 0.05080545 |
| cg05102552 | 0.14787278 |
| cg05152300 | 0.85997522 |
| cg05155047 | 0.98389095 |
| cg05179172 | 0.52664188 |
| cg05248542 | 0.51548947 |
| cg05265042 | −8.9118833 |
| cg05265359 | 0.42420487 |
| cg05280698 | 0.08674102 |
| cg05295671 | 0.12432879 |
| cg05309877 | −1.4987288 |
| cg05324407 | 0.12224079 |
| cg05331334 | −0.8602928 |
| cg05355167 | 0.86782321 |
| cg05360774 | 0.28004957 |
| cg05376617 | 0.08963238 |
| cg05386977 | 0.63279637 |
| cg05388545 | 0.93102024 |
| cg05388821 | −2.0595942 |
| cg05391998 | 0.41511772 |
| cg05445326 | −0.1731712 |
| cg05463027 | −29.740548 |
| cg05483252 | 0.30524577 |
| cg05521150 | 0.70218918 |
| cg05577016 | 0.29904998 |
| cg05593641 | 0.03841388 |
| cg05597836 | 0.46261875 |
| cg05656900 | 0.75505989 |
| cg05726118 | 0.05163156 |
| cg05765580 | 0.13052458 |
| cg05767404 | 0.5633686 |
| cg05770238 | 0.24204874 |
| cg05774698 | −2.3206316 |
| cg05829145 | 0.17059067 |
| cg05861879 | −5.5436469 |
| cg05863683 | −0.2832814 |
| cg05869537 | 0.06361008 |
| cg05896902 | 0.21520033 |
| cg05980111 | −5.5225434 |
| cg05991454 | 0.01404378 |
| cg06024411 | 0.04295746 |
| cg06073139 | −1.2880606 |
| cg06120399 | 0.73853779 |
| cg06145435 | −0.6771877 |
| cg06147863 | 0.37752995 |
| cg06156376 | 0.27344073 |
| cg06182099 | 0.94630318 |
| cg06204938 | 0.5377943 |
| cg06217245 | −2.2781315 |
| cg06235390 | 0.51218505 |
| cg06411551 | 0.09706733 |
| cg06460691 | −4.0771901 |
| cg06473578 | 0.36637753 |
| cg06490845 | 0.18215613 |
| cg06504636 | 1.3816152 |
| cg06522772 | 0.72821148 |
| cg06545268 | 0.11565469 |
| cg06565975 | −1.3893524 |
| cg06594770 | 0.30028914 |
| cg06619299 | −1.6237109 |
| cg06625004 | 0.14126394 |
| cg06639733 | −1.0880244 |
| cg06644488 | 0.3866171 |
| cg06660332 | 0.28087567 |
| cg06664254 | 0.38124742 |
| cg06682875 | 0.23973731 |
| cg06697600 | 0.33002891 |
| cg06712651 | 0.9913259 |
| cg06713116 | −5.0008951 |
| cg06723492 | −1.8510846 |
| cg06732989 | −1.1033003 |
| cg06734510 | −3.6683441 |
| cg06754224 | 0.67038414 |
| cg06772202 | 0.57553486 |
| cg06799422 | 0.3283767 |
| cg06807593 | 0.88021479 |
| cg06817264 | 0.05700124 |
| cg06867482 | 0.54233788 |
| cg06868100 | 0.45724907 |
| cg06871074 | 0.22800496 |
| cg06872257 | 0.62866584 |
| cg06872548 | 0.2432879 |
| cg06882058 | 0.48409748 |
| cg06891458 | 0.21974391 |
| cg06916725 | 0.00454358 |
| cg06933824 | 0.25609252 |
| cg06980387 | 0.02808757 |
| cg06995548 | 0.32796365 |
| cg07000567 | 0.35952108 |
| cg07029024 | 0.874019 |
| cg07097041 | 0.38950847 |
| cg07104557 | 0.34151336 |
| cg07163735 | −7.3749949 |
| cg07170253 | −5.8797734 |
| cg07200877 | 0.94052045 |
| cg07216884 | 0.53366378 |
| cg07235774 | 0.29574556 |
| cg07235805 | 0.49194548 |
| cg07240834 | 0.2094176 |
| cg07255019 | 0.70962412 |
| cg07286682 | 0.03593556 |
| cg07312601 | −10.346276 |
| cg07322898 | 0.11566731 |
| cg07325246 | 0.27385378 |
| cg07379055 | 0.21354812 |
| cg07379335 | 0.67988435 |
| cg07384080 | 0.39859562 |
| cg07393255 | 0.09830648 |
| cg07401435 | −7.3437923 |
| cg07434944 | 0.35333896 |
| cg07447773 | 0.26724494 |
| cg07486199 | −3.7348837 |
| cg07495704 | 23.247216 |
| cg07537152 | −0.4074002 |
| cg07540084 | 0.53283767 |
| cg07547765 | 0.39363899 |
| cg07560510 | 0.15365551 |
| cg07571344 | 0.38496489 |
| cg07571928 | 0.27013631 |
| cg07590529 | 0.20280876 |
| cg07671586 | 0.32218092 |
| cg07675337 | 0.39983478 |
| cg07725123 | 0.40066088 |
| cg07736657 | −0.566537 |
| cg07742235 | 0.67677982 |
| cg07764386 | −7.3464167 |
| cg07779444 | 0.42337877 |
| cg07800658 | 0.58653449 |
| cg07803375 | 0.49896737 |
| cg07833467 | −0.4036801 |
| cg07850154 | −33.649864 |
| cg07888957 | 0.19248245 |
| cg07910813 | −2.5746778 |
| cg07917528 | 0.07369633 |
| cg07925670 | 0.33952912 |
| cg07938847 | −2.6650278 |
| cg07961015 | 0.36596448 |
| cg08030082 | 0.26559273 |
| cg08034070 | 0.22635275 |
| cg08034171 | 0.16480793 |
| cg08046569 | 0.17554729 |
| cg08081725 | −16.501516 |
| cg08096291 | 0.37505163 |
| cg08129490 | 0.82321355 |
| cg08203715 | 0.79471293 |
| cg08208133 | 0.37918216 |
| cg08220614 | 0.14327503 |
| cg08235413 | −10.475345 |
| cg08241514 | 0.4370095 |
| cg08248579 | 0.96860801 |
| cg08270964 | 0.96158612 |
| cg08274097 | 0.99256506 |
| cg08277216 | 0.34944238 |
| cg08301612 | −10.993374 |
| cg08317738 | 0.10097237 |
| cg08349335 | 0.89384552 |
| cg08373610 | 0.97315159 |
| cg08402963 | 0.32094176 |
| cg08428188 | 0.622057 |
| cg08434127 | −1.8528728 |
| cg08443203 | 0.47459727 |
| cg08467103 | 0.09845415 |
| cg08526814 | −24.671921 |
| cg08529529 | −27.936793 |
| cg08530484 | −1.030583 |
| cg08551532 | 0.54702784 |
| cg08583763 | 0.81825692 |
| cg08593364 | −17.422289 |
| cg08614441 | 0.62701363 |
| cg08644365 | −0.5085344 |
| cg08649707 | −5.2382722 |
| cg08652441 | 0.37257332 |
| cg08663634 | 0.43205287 |
| cg08693738 | 0.02147873 |
| cg08723357 | 0.98513011 |
| cg08733522 | −7.2801308 |
| cg08742502 | 0.98926064 |
| cg08749599 | 0.96076002 |
| cg08762484 | 0.67905824 |
| cg08764927 | 0.32259397 |
| cg08797444 | 0.22470054 |
| cg08822136 | 0.20735233 |
| cg08824847 | 0.65551425 |
| cg08826281 | −2.1134789 |
| cg08844900 | −2.6351629 |
| cg08858130 | 0.08880628 |
| cg08878450 | 0.32266299 |
| cg09012544 | 0.07063197 |
| cg09025327 | 0.47583643 |
| cg09042411 | 0.20652623 |
| cg09053247 | 0.25145267 |
| cg09063262 | −3.913876 |
| cg09110394 | 0.7306898 |
| cg09119665 | −4.0774313 |
| cg09134314 | 0.93638992 |
| cg09143673 | 0.377943 |
| cg09151131 | 0.87980173 |
| cg09153897 | 0.15489467 |
| cg09164168 | 0.20693928 |
| cg09206294 | 0.34696406 |
| cg09234599 | 0.81990913 |
| cg09294095 | 0.16026435 |
| cg09323728 | 0.42668319 |
| cg09328979 | 0.31722429 |
| cg09363587 | 0.49938042 |
| cg09425279 | 0.16067741 |
| cg09428868 | 0.83767038 |
| cg09521743 | 0.10574143 |
| cg09547190 | 0.64229657 |
| cg09578829 | 0.37339942 |
| cg09628195 | 0.48161917 |
| cg09645572 | 0.31882971 |
| cg09728393 | 0.99008674 |
| cg09754948 | −15.802845 |
| cg09773473 | 0.47418422 |
| cg09832613 | −1.4748338 |
| cg09837656 | 0.60718711 |
| cg09854088 | 0.4952499 |
| cg09884146 | 0.93473771 |
| cg09924848 | 0.7315159 |
| cg09938213 | 0.29533251 |
| cg09948192 | −0.7247699 |
| cg09969462 | 0.36018174 |
| cg10046620 | 0.15737299 |
| cg10082647 | 0.19578686 |
| cg10110957 | 0.95704254 |
| cg10123952 | −1.1268934 |
| cg10133725 | 0.47914085 |
| cg10147507 | 0.16811235 |
| cg10149296 | 0.35196017 |
| cg10196532 | 0.07724081 |
| cg10213353 | 0.42255266 |
| cg10314221 | −3.862696 |
| cg10378538 | −0.6561886 |
| cg10395519 | 0.54935977 |
| cg10406027 | −9.6433505 |
| cg10426464 | 0.46840149 |
| cg10432859 | 0.59438249 |
| cg10460946 | 0.71623296 |
| cg10515671 | −8.3004843 |
| cg10519437 | 0.03444374 |
| cg10529555 | −6.4830674 |
| cg10543574 | 0.10698059 |
| cg10599571 | 0.26352747 |
| cg10612617 | 0.02933541 |
| cg10616300 | 0.27426683 |
| cg10662179 | 0.25898389 |
| cg10693071 | 0.04832714 |
| cg10695490 | −4.952111 |
| cg10715265 | 0.17141677 |
| cg10741153 | 0.50309789 |
| cg10750934 | −6.1174203 |
| cg10760299 | 0.23874432 |
| cg10770076 | 0.49814126 |
| cg10780778 | −1.5245162 |
| cg10805511 | 0.118133 |
| cg10809491 | 0.1842214 |
| cg10836173 | 0.2180917 |
| cg10919204 | 0.4291615 |
| cg10951117 | −8.2730564 |
| cg10960709 | 0.10656753 |
| cg11015497 | −2.1695284 |
| cg11029475 | 0.40603057 |
| cg11155735 | 0.14806705 |
| cg11173499 | 0.18746666 |
| cg11177223 | 0.7653862 |
| cg11218175 | −1.0592031 |
| cg11290188 | 0.46798843 |
| cg11313708 | 0.20033044 |
| cg11358741 | 0.0842627 |
| cg11402700 | 0.16398183 |
| cg11421702 | 0.95993391 |
| cg11438134 | −4.094798 |
| cg11449408 | −1.1052523 |
| cg11471262 | 0.99752169 |
| cg11505841 | 0.37587774 |
| cg11517269 | 0.3738276 |
| cg11534593 | 0.29120198 |
| cg11540735 | 0.26807105 |
| cg11562411 | 0.1771995 |
| cg11565355 | 0.08054523 |
| cg11618577 | 0.13630731 |
| cg11663600 | 0.0243701 |
| cg11756095 | 0.71045023 |
| cg11792186 | −0.1382859 |
| cg11829633 | 0.91325898 |
| cg11835020 | 0.96034696 |
| cg11835347 | −13.517496 |
| cg11840849 | −3.4640898 |
| cg11934819 | 0.37174721 |
| cg12000995 | 0.23296159 |
| cg12007048 | −10.527821 |
| cg12036877 | 0.52413672 |
| cg12045999 | 0.18959108 |
| cg12051614 | 0.31350682 |
| cg12058385 | −0.0018232 |
| cg12119029 | 0.44444444 |
| cg12122631 | 0.54316398 |
| cg12131894 | 2.3862026 |
| cg12133664 | 0.57290376 |
| cg12188416 | −0.7559321 |
| cg12205435 | 0.15184526 |
| cg12211856 | 0.38083437 |
| cg12223258 | 0.13878563 |
| cg12226009 | −0.4107873 |
| cg12283398 | 0.11482858 |
| cg12305200 | 0.05534903 |
| cg12325455 | 0.25526642 |
| cg12354986 | −4.2564486 |
| cg12380854 | 0.73027675 |
| cg12459028 | 0.73275506 |
| cg12480416 | 0.22552664 |
| cg12491223 | −12.299201 |
| cg12580930 | 0.81206113 |
| cg12581592 | 0.24576621 |
| cg12594615 | 0.45105328 |
| cg12596182 | 0.71747212 |
| cg12606409 | 0.10037175 |
| cg12750151 | 0.90251962 |
| cg12753009 | 0.51135894 |
| cg12788037 | 0.19041718 |
| cg12836280 | 0.30607187 |
| cg13000649 | 0.16191656 |
| cg13017983 | 0.72490706 |
| cg13058214 | 0.70838496 |
| cg13061373 | −1.0841955 |
| cg13079123 | 0.41676993 |
| cg13127159 | −1.9245147 |
| cg13127231 | 0.2779843 |
| cg13139020 | −0.8313673 |
| cg13156931 | 0.51466336 |
| cg13227806 | 0.15595552 |
| cg13283153 | 0.14085089 |
| cg13311096 | 0.25030979 |
| cg13323701 | 0.3527468 |
| cg13330671 | 0.1138824 |
| cg13390332 | −9.4188609 |
| cg13393036 | 0.38042131 |
| cg13399816 | 0.12102437 |
| cg13417559 | 0.94134655 |
| cg13435820 | 0.53903346 |
| cg13507964 | 0.40892193 |
| cg13549152 | 0.58777365 |
| cg13557773 | −0.5718858 |
| cg13561879 | 0.01528294 |
| cg13569146 | 0.15324246 |
| cg13582001 | 0.4361834 |
| cg13666174 | −0.2186323 |
| cg13687915 | 0.9991739 |
| cg13690424 | 0.1598513 |
| cg13721134 | 0.19619992 |
| cg13731523 | 0.13768293 |
| cg13732582 | 0.53077241 |
| cg13777609 | −1.9780738 |
| cg13790268 | 0.49731516 |
| cg13791379 | −3.1212753 |
| cg13792233 | 0.78769104 |
| cg13798745 | 0.3465923 |
| cg13813086 | −6.2266195 |
| cg13827984 | 0.5464684 |
| cg13831329 | 0.40644362 |
| cg13868473 | 0.40850888 |
| cg13872065 | 0.85790995 |
| cg13947929 | 0.04460967 |
| cg13953458 | 0.09500207 |
| cg14003022 | −1.3985372 |
| cg14053997 | −0.1735621 |
| cg14067761 | 0.8566708 |
| cg14074486 | 0.39033457 |
| cg14198472 | 0.23915737 |
| cg14228146 | 0.26765799 |
| cg14242246 | 0.83188765 |
| cg14268226 | 0.81123503 |
| cg14268632 | 0.24494011 |
| cg14353201 | −2.4924204 |
| cg14363469 | 0.4118133 |
| cg14368149 | 0.9834779 |
| cg14371590 | 0.51920694 |
| cg14388049 | 0.19206939 |
| cg14442518 | 0.98182569 |
| cg14524754 | 0.22511359 |
| cg14540297 | −9.9491088 |
| cg14584255 | 0.35609365 |
| cg14591667 | 0.13424205 |
| cg14594111 | 0.95456423 |
| cg14602471 | 0.52953325 |
| cg14634687 | 0.92688971 |
| cg14688451 | −3.9222755 |
| cg14692106 | 0.25691863 |
| cg14701072 | 0.44286149 |
| cg14757738 | 0.25981 |
| cg14768256 | −0.9030108 |
| cg14775751 | −3.5926636 |
| cg14781189 | 0.38455184 |
| cg14848077 | −2.6341313 |
| cg14903689 | 0.67781908 |
| cg14905600 | 0.76290789 |
| cg14919250 | 0.21685254 |
| cg14939821 | 0.64849236 |
| cg15031579 | 0.94589013 |
| cg15038286 | −3.5037606 |
| cg15063695 | −14.618653 |
| cg15102179 | 0.51011979 |
| cg15105011 | −0.5760175 |
| cg15232290 | 0.33587858 |
| cg15247329 | −0.2113557 |
| cg15258447 | 0.27302767 |
| cg15262984 | −1.7429628 |
| cg15282632 | 0.629905 |
| cg15299997 | −13.147995 |
| cg15373880 | 0.86947542 |
| cg15384383 | 0.32011565 |
| cg15397472 | 0.33663775 |
| cg15409712 | 0.57703428 |
| cg15431821 | 0.21643949 |
| cg15445281 | 0.02886918 |
| cg15543489 | −0.0346824 |
| cg15575356 | 0.88682363 |
| cg15600051 | −1.3099326 |
| cg15604051 | 0.43907476 |
| cg15611336 | 0.15076415 |
| cg15626112 | 0.5125981 |
| cg15635368 | 0.88351921 |
| cg15639684 | 0.14002478 |
| cg15662902 | 0.1425031 |
| cg15686393 | 0.06113176 |
| cg15706250 | 0.19991739 |
| cg15744005 | 0.35729038 |
| cg15751090 | 0.18793887 |
| cg15771128 | −6.1777319 |
| cg15792487 | 0.01363073 |
| cg15824291 | 0.30070219 |
| cg15840418 | −0.0430472 |
| cg15876676 | 0.39818257 |
| cg15922176 | 0.92317224 |
| cg15964523 | 0.24989674 |
| cg15988970 | −0.1499066 |
| cg15996342 | 0.19950434 |
| cg16010596 | 0.17802561 |
| cg16139227 | 0.35770343 |
| cg16151795 | 0.36472532 |
| cg16178415 | 0.64394878 |
| cg16195091 | 0.14622057 |
| cg16213375 | 0.01776126 |
| cg16218715 | −9.0936119 |
| cg16248756 | 0.63651384 |
| cg16251130 | −1.5017749 |
| cg16296679 | 0.5195628 |
| cg16308533 | 0.54275093 |
| cg16326902 | 0.29987608 |
| cg16335858 | 0.85501859 |
| cg16368750 | 0.10739364 |
| cg16375265 | −3.3112668 |
| cg16399833 | 0.73895085 |
| cg16427513 | −2.0526409 |
| cg16448636 | 0.52209831 |
| cg16457307 | −0.6104153 |
| cg16520312 | 0.03882693 |
| cg16555466 | 0.87443205 |
| cg16572224 | 0.76724479 |
| cg16596957 | −0.9858203 |
| cg16643422 | 0.69516729 |
| cg16653408 | 0.83973565 |
| cg16699385 | 0.44155308 |
| cg16701167 | 0.22428748 |
| cg16751098 | 1.081061 |
| cg16824126 | 0.04956629 |
| cg16845257 | −0.4526925 |
| cg16861209 | 0.17472119 |
| cg16886581 | 0.24080958 |
| cg16888547 | −7.6065654 |
| cg16895261 | 0.28954977 |
| cg16931969 | 0.06195787 |
| cg16936289 | 0.9417596 |
| cg16949914 | 0.9582817 |
| cg16983588 | −8.6006096 |
| cg17053538 | −1.6703483 |
| cg17054674 | −8.427154 |
| cg17088155 | −0.4569773 |
| cg17105886 | 0.95415118 |
| cg17109725 | 0.4978282 |
| cg17173187 | 0.14952499 |
| cg17179570 | 0.16935151 |
| cg17230535 | 0.41924824 |
| cg17255214 | 0.43866171 |
| cg17274064 | −5.7760618 |
| cg17279125 | 0.04667493 |
| cg17279458 | 0.48864106 |
| cg17310773 | −5.8338111 |
| cg17319774 | 0.04130525 |
| cg17327990 | 0.29822387 |
| cg17334937 | 0.51301115 |
| cg17430167 | 0.11028501 |
| cg17491146 | 0.36183395 |
| cg17494199 | −3.8229739 |
| cg17521665 | 0.98967369 |
| cg17527798 | 0.47170591 |
| cg17587327 | 0.22924411 |
| cg17598574 | 0.39942173 |
| cg17667648 | 0.32465923 |
| cg17672850 | 0.0691232 |
| cg17708016 | 0.44981413 |
| cg17814814 | 0.2850062 |
| cg17852385 | 0.24617926 |
| cg17870909 | −8.312084 |
| cg17877566 | 0.79223461 |
| cg17910899 | −0.0708525 |
| cg17951878 | −3.9841042 |
| cg17968037 | 0.4543577 |
| cg17996830 | −0.6127867 |
| cg18034295 | 0.61296985 |
| cg18059933 | 0.39487815 |
| cg18064071 | −0.4993625 |
| cg18070470 | 0.26022305 |
| cg18071071 | −0.5231766 |
| cg18161890 | 0.39116068 |
| cg18222590 | 0.78686493 |
| cg18245230 | 0.8409748 |
| cg18257485 | 0.82651797 |
| cg18297745 | 0.08467172 |
| cg18320111 | 0.28748451 |
| cg18329931 | 0.47666254 |
| cg18346576 | 0.97852127 |
| cg18365211 | −5.0122056 |
| cg18374181 | 0.24452705 |
| cg18385671 | −0.6639838 |
| cg18419358 | 0.90541099 |
| cg18449021 | 0.19867823 |
| cg18468088 | 0.20363486 |
| cg18477009 | 0.41003896 |
| cg18497052 | 0.19124329 |
| cg18515886 | −2.7680745 |
| cg18538662 | 0.30194135 |
| cg18552861 | 0.28624535 |
| cg18581929 | 0.15778604 |
| cg18611122 | 0.74266832 |
| cg18625610 | 0.47046675 |
| cg18634665 | 0.17265593 |
| cg18638383 | −2.7074454 |
| cg18668382 | 0.88104089 |
| cg18674980 | 0.13093763 |
| cg18696495 | 0.65097067 |
| cg18735810 | 0.11648079 |
| cg18737081 | 0.74060306 |
| cg18793806 | 0.31928955 |
| cg18797590 | −8.2394651 |
| cg18811731 | 0.58157786 |
| cg18833928 | 0.49979347 |
| cg18894440 | 0.38372573 |
| cg18931760 | 0.39239983 |
| cg18940274 | 0.9574556 |
| cg18958126 | 0.04874019 |
| cg19002763 | −0.0251517 |
| cg19008597 | 0.75919042 |
| cg19013753 | 0.17843866 |
| cg19021188 | 0.08937578 |
| cg19043574 | 0.06071871 |
| cg19065831 | 0.30400661 |
| cg19066391 | 0.35852953 |
| cg19196221 | 0.2276792 |
| cg19197212 | 0.5212722 |
| cg19205909 | 0.06567534 |
| cg19211382 | 0.755886 |
| cg19226100 | 0.98099959 |
| cg19248564 | 0.41057414 |
| cg19308132 | 0.45229244 |
| cg19324714 | 0.39694341 |
| cg19445684 | 0.61941408 |
| cg19452535 | 0.43494424 |
| cg19462210 | −1.5097708 |
| cg19506311 | 0.70590665 |
| cg19514613 | 0.00123916 |
| cg19539664 | −12.634464 |
| cg19552640 | −1.689361 |
| cg19570154 | 0.26311442 |
| cg19685479 | 0.05989261 |
| cg19691410 | −5.8936844 |
| cg19697725 | 0.89591078 |
| cg19716643 | 0.46881454 |
| cg19733534 | 0.49690211 |
| cg19761014 | 0.97810822 |
| cg19774627 | 0.20322181 |
| cg19777853 | 0.43081371 |
| cg19830657 | 0.1511772 |
| cg19857407 | 0.07269723 |
| cg19900821 | −1.6836541 |
| cg19904425 | 0.15530772 |
| cg19935065 | 0.47335812 |
| cg19959917 | 0.4204874 |
| cg19965810 | 0.07228418 |
| cg20014988 | 0.46964064 |
| cg20039814 | 0.43742255 |
| cg20059012 | −4.2804306 |
| cg20102877 | 0.1007848 |
| cg20110742 | −9.9078836 |
| cg20120351 | 0.90458488 |
| cg20141652 | 0.10987195 |
| cg20155447 | −0.5916918 |
| cg20172563 | 0.37959521 |
| cg20203395 | 0.38166047 |
| cg20235117 | −9.2745905 |
| cg20262330 | 0.29698472 |
| cg20271057 | 0.60019543 |
| cg20276402 | −0.3678528 |
| cg20320656 | 0.92647666 |
| cg20321251 | 0.95786865 |
| cg20353653 | 0.92895498 |
| cg20356878 | −0.0268829 |
| cg20433521 | 0.48120611 |
| cg20454518 | 0.13919868 |
| cg20456258 | 0.23904239 |
| cg20468787 | −1.3703523 |
| cg20482143 | −2.59662 |
| cg20494635 | −4.3340389 |
| cg20623702 | 0.88723668 |
| cg20631820 | −0.1464032 |
| cg20642413 | 0.74225527 |
| cg20666917 | 0.0582404 |
| cg20701183 | 0.25939694 |
| cg20708173 | 0.4109872 |
| cg20711218 | 0.0417183 |
| cg20713174 | 0.1268071 |
| cg20744163 | 0.15029271 |
| cg20775810 | −0.9542863 |
| cg20780880 | 0.08921933 |
| cg20790367 | 0.43246592 |
| cg20797905 | 0.53159851 |
| cg20802392 | 0.79677819 |
| cg20816447 | −10.015135 |
| cg20893838 | 0.28046262 |
| cg20908204 | 0.78479967 |
| cg20991421 | 0.37835605 |
| cg21004924 | 0.10326311 |
| cg21052677 | 0.80503924 |
| cg21064451 | 0.881377 |
| cg21088119 | −4.9299573 |
| cg21112954 | 0.40933499 |
| cg21136371 | 0.71086328 |
| cg21144340 | −0.4914244 |
| cg21188242 | 0.82693102 |
| cg21207665 | 0.39405204 |
| cg21248554 | 0.11317637 |
| cg21251926 | −1.5289413 |
| cg21293242 | 0.2606361 |
| cg21320768 | 0.03634862 |
| cg21333674 | −2.0023163 |
| cg21357291 | 0.41429162 |
| cg21415227 | 0.46509707 |
| cg21436456 | 0.28170178 |
| cg21479132 | −2.6510691 |
| cg21500300 | 0.74142916 |
| cg21500966 | 0.12143742 |
| cg21571060 | −7.4123425 |
| cg21574853 | −7.3866885 |
| cg21644387 | 0.67327551 |
| cg21672276 | 0.4535316 |
| cg21692450 | 0.01407262 |
| cg21697134 | −1.790877 |
| cg21759268 | 0.28211483 |
| cg21782813 | −4.3474898 |
| cg21793437 | 0.94382487 |
| cg21796167 | −11.863581 |
| cg21838488 | 0.11441553 |
| cg21839331 | 0.07551636 |
| cg21854228 | −0.2803682 |
| cg21923770 | 0.71750848 |
| cg21926804 | −0.3900075 |
| cg21946667 | 0.34035523 |
| cg21962450 | 0.1094589 |
| cg22012583 | 0.87608426 |
| cg22022379 | 0.48203222 |
| cg22025854 | −1.8754215 |
| cg22027946 | 0.4842573 |
| cg22079161 | 0.38248658 |
| cg22103003 | −2.9167224 |
| cg22120714 | 0.00619579 |
| cg22156842 | −2.3737996 |
| cg22189725 | 0.09624122 |
| cg22202381 | 0.31598513 |
| cg22239201 | 0.99586948 |
| cg22242614 | −3.9214824 |
| cg22264409 | 0.39281289 |
| cg22283925 | 0.43411813 |
| cg22348356 | −10.320885 |
| cg22425568 | 0.11400248 |
| cg22548220 | 0.23420074 |
| cg22637538 | 0.01693515 |
| cg22639561 | −9.5440652 |
| cg22652782 | −18.139696 |
| cg22706610 | 0.50351095 |
| cg22720431 | −0.0843688 |
| cg22733207 | 0.87897563 |
| cg22737282 | 0.00536968 |
| cg22779878 | −3.7872326 |
| cg22826874 | 0.20487402 |
| cg22860775 | 0.50846758 |
| cg22900229 | 0.36885584 |
| cg22920538 | −3.8021331 |
| cg22935921 | 0.38744321 |
| cg22977317 | 0.79595209 |
| cg23030863 | 0.46179265 |
| cg23043611 | 0.39570425 |
| cg23065100 | −0.5640633 |
| cg23066280 | −0.1532217 |
| cg23080060 | −1.922041 |
| cg23124451 | −13.492188 |
| cg23151014 | 0.51425031 |
| cg23172400 | 0.41016109 |
| cg23188704 | 0.51590252 |
| cg23206745 | −0.4351523 |
| cg23207054 | 0.93143329 |
| cg23210521 | 0.03180504 |
| cg23260525 | −0.5572306 |
| cg23266598 | −10.31931 |
| cg23282585 | −16.240948 |
| cg23307798 | 0.08302354 |
| cg23336797 | 0.55183808 |
| cg23367683 | 0.15819909 |
| cg23373153 | −3.6167741 |
| cg23464183 | 0.31268071 |
| cg23489630 | 0.52168525 |
| cg23581183 | 0.7984304 |
| cg23581793 | 0.86327964 |
| cg23600866 | 0.26228831 |
| cg23613051 | −0.131812 |
| cg23618638 | 0.19496076 |
| cg23624713 | −5.5455047 |
| cg23626546 | −7.6915671 |
| cg23670519 | 0.72903759 |
| cg23698023 | −0.0562295 |
| cg23736055 | −4.5656728 |
| cg23737061 | 0.87938868 |
| cg23737927 | −0.6394294 |
| cg23777173 | 0.8476144 |
| cg23799375 | 0.2771582 |
| cg23830205 | −3.296932 |
| cg23833896 | 1.8355147 |
| cg23893460 | 0.23667906 |
| cg23895495 | 0.62247005 |
| cg24015175 | 0.99339116 |
| cg24044052 | −5.4747311 |
| cg24087736 | 0.10473543 |
| cg24109012 | 0.51886534 |
| cg24112692 | 0.26187526 |
| cg24164702 | 0.7645601 |
| cg24240870 | −0.5041117 |
| cg24249248 | −1.2904337 |
| cg24253500 | 0.11832422 |
| cg24311135 | 0.34985543 |
| cg24327262 | 0.53820735 |
| cg24370881 | 0.48616274 |
| cg24412006 | 0.71581991 |
| cg24437311 | −6.4799017 |
| cg24453699 | 0.48905411 |
| cg24479590 | 0.00743494 |
| cg24512005 | 0.61462206 |
| cg24555670 | 0.26517968 |
| cg24617723 | 0.09582817 |
| cg24650267 | 0.55762082 |
| cg24674269 | 0.45477076 |
| cg24757926 | 0.52829409 |
| cg24768116 | 0.10904585 |
| cg24784350 | 0.08715407 |
| cg24870774 | 0.00165221 |
| cg24873872 | 0.35277505 |
| cg24874254 | 0.98017348 |
| cg24913868 | −10.011699 |
| cg24920358 | 0.41635688 |
| cg24924449 | 0.60760017 |
| cg24935556 | −2.8696772 |
| cg24952754 | −7.5999228 |
| cg24983539 | 0.84262701 |
| cg25036456 | −2.4128653 |
| cg25119002 | −0.2462162 |
| cg25122125 | −4.4430393 |
| cg25123427 | 0.79347377 |
| cg25127315 | 0.12928542 |
| cg25151919 | 0.49318463 |
| cg25179758 | 0.91615035 |
| cg25188760 | 0.20948276 |
| cg25215230 | −0.8463894 |
| cg25326896 | 0.00660884 |
| cg25351599 | 0.11358943 |
| cg25353171 | 0.32548534 |
| cg25361506 | 0.26972325 |
| cg25363789 | −1.576559 |
| cg25394782 | 0.12725713 |
| cg25409140 | 0.23213548 |
| cg25481454 | 0.92028088 |
| cg25486749 | −1.803531 |
| cg25509697 | 0.38826931 |
| cg25561904 | 0.35687732 |
| cg25645064 | 0.04419661 |
| cg25660036 | 0.24039653 |
| cg25671438 | 0.72779843 |
| cg25697881 | −0.4122874 |
| cg25749107 | 0.00927456 |
| cg25753817 | 0.88888889 |
| cg25783326 | 0.24370095 |
| cg25846723 | 0.3692689 |
| cg25860399 | −0.9647031 |
| cg25872744 | 0.40520446 |
| cg25940248 | 0.55018587 |
| cg25961618 | 0.77282115 |
| cg25969992 | −0.2306091 |
| cg26035892 | 0.71953738 |
| cg26070099 | 0.02560925 |
| cg26082368 | 0.22098306 |
| cg26097391 | 0.53448988 |
| cg26157803 | 0.51714168 |
| cg26160218 | 0.30701417 |
| cg26175729 | 0.83932259 |
| cg26282236 | 0.42021274 |
| cg26292895 | −8.5276601 |
| cg26307871 | 0.35481206 |
| cg26317006 | 0.92854192 |
| cg26322872 | −2.3475033 |
| cg26325335 | 0.21957404 |
| cg26340700 | 0.77075589 |
| cg26365925 | 0.06030566 |
| cg26397549 | −6.2140885 |
| cg26403416 | −0.9597117 |
| cg26424649 | 0.2684841 |
| cg26467949 | −1.7064785 |
| cg26468833 | 0.60388269 |
| cg26471982 | 0.63527468 |
| cg26493814 | −0.8158753 |
| cg26509915 | −0.1443512 |
| cg26514961 | 0.29491945 |
| cg26562921 | 0.54068567 |
| cg26635214 | −15.180625 |
| cg26636010 | −0.7634238 |
| cg26684673 | 0.25237505 |
| cg26693467 | −4.0957978 |
| cg26726141 | −2.9017718 |
| cg26734888 | 0.30978934 |
| cg26780581 | 0.03469641 |
| cg26781129 | 0.48244527 |
| cg26808167 | 0.59231722 |
| cg26848071 | 0.86245353 |
| cg26850624 | −0.2749982 |
| cg26888530 | 0.20983065 |
| cg26951091 | 0.9487815 |
| cg27021512 | −12.094373 |
| cg27022827 | 0.74019 |
| cg27039118 | 0.96819496 |
| cg27045062 | −6.4033385 |
| cg27078652 | −3.4844279 |
| cg27227029 | 0.20528707 |
| cg27241134 | −5.5578509 |
| cg27249858 | 0.88847584 |
| cg27261733 | 0.36100785 |
| cg27262717 | 0.18876497 |
| cg27300045 | −2.7594005 |
| cg27355653 | −0.2932958 |
| cg27396824 | 0.25825019 |
| cg27434984 | 0.98719537 |
| cg27532318 | 0.4548635 |
| cg27574654 | 0.32300702 |
| cg27629782 | −1.1434961 |
| cg27637363 | 0.04584882 |
| cg27645544 | −0.210304 |

TABLE C
AdaptAge CpG Sites and Estimates

| term | estimate |
|---|---|
| (Intercept) | −511.97428 |
| cg00008671 | −0.0137092 |
| cg00017970 | −0.0001084 |
| cg00048759 | 33.8029569 |
| cg00050402 | −0.0494625 |
| cg00089550 | −0.0260158 |
| cg00099240 | −0.000155 |
| cg00108164 | 16.0467868 |
| cg00131893 | 1.96264444 |
| cg00158122 | −0.000292 |
| cg00223715 | 0.58293085 |
| cg00229508 | −7.096E−05 |
| cg00277334 | −0.001087 |
| cg00290758 | −6.861E−05 |
| cg00295744 | 1.42084533 |
| cg00316485 | −0.0016145 |
| cg00335735 | −5.526E−05 |
| cg00342891 | −0.0037653 |
| cg00344422 | −0.0001112 |
| cg00346145 | 3.4042617 |
| cg00388262 | −0.0002657 |
| cg00492070 | −0.0003776 |
| cg00505045 | 7.50388563 |
| cg00513984 | −9.961E−05 |
| cg00539174 | 0.08913463 |
| cg00544337 | −0.0071478 |
| cg00552753 | −0.0005405 |
| cg00561903 | −0.0001166 |
| cg00577578 | −0.0004865 |
| cg00589581 | −0.0112065 |
| cg00638945 | −0.0159653 |
| cg00655982 | 0.0052405 |
| cg00712841 | −0.0017416 |
| cg00715290 | −5.667E−05 |
| cg00750088 | −0.000271 |
| cg00785170 | −0.0011901 |
| cg00834400 | −0.0003867 |
| cg00851050 | −0.0002258 |
| cg00859280 | −0.0003513 |
| cg00870514 | −0.0008508 |
| cg00877329 | −0.0001899 |
| cg00910168 | −4.542E−05 |
| cg00929523 | −0.0001944 |
| cg00933182 | −0.0002079 |
| cg01019770 | −0.0411924 |
| cg01048752 | −0.0071107 |
| cg01055594 | −0.0001758 |
| cg01065599 | −0.0008653 |
| cg01080986 | 15.2784519 |
| cg01081263 | −0.0001901 |
| cg01103582 | 9.91943444 |
| cg01181940 | −9.093E−05 |
| cg01192291 | 0.0015668 |
| cg01209296 | −0.000947 |
| cg01213022 | 6.54763092 |
| cg01229452 | −0.0246304 |
| cg01239922 | −0.0053352 |
| cg01245393 | 0.0002436 |
| cg01262865 | −7.843E−05 |
| cg01274524 | 4.98655078 |
| cg01307174 | −0.0001952 |
| cg01346077 | −0.0003159 |
| cg01399860 | −9.363E−05 |
| cg01416891 | −0.0003488 |
| cg01421252 | −0.000279 |
| cg01433677 | −0.000813 |
| cg01521220 | 0.0186156 |
| cg01530283 | −8.867E−05 |
| cg01534887 | 1.82962067 |
| cg01538166 | −0.0088581 |
| cg01544580 | −0.0008566 |
| cg01563071 | 2.97887625 |
| cg01579218 | −0.0006783 |
| cg01595397 | −8.643E−05 |
| cg01611548 | −0.0007084 |
| cg01614478 | 0.73435215 |
| cg01641620 | −0.0002175 |
| cg01647632 | −0.0004737 |
| cg01676795 | −0.0441272 |
| cg01678292 | 3.16798757 |
| cg01686177 | 2.03776505 |
| cg01707820 | −0.0016673 |
| cg01768926 | −0.0031455 |
| cg01783841 | −9.586E−05 |
| cg01785233 | −3.11E−05 |
| cg01787798 | −0.000104 |
| cg01813672 | −0.0002286 |
| cg01877606 | −0.0002196 |
| cg01899318 | −0.0001192 |
| cg01943692 | −0.0001723 |
| cg02010447 | −1.964E−05 |
| cg02061130 | −7.491E−05 |
| cg02061804 | 0.71145756 |
| cg02071712 | −0.0001582 |
| cg02079584 | −0.0002448 |
| cg02131130 | −0.0013704 |
| cg02133624 | −3.241E−05 |
| cg02145668 | −0.0033739 |
| cg02161761 | 1.74744658 |
| cg02186748 | −0.0026756 |
| cg02216481 | −2.701E−05 |
| cg02225085 | 10.3729591 |
| cg02264895 | −0.0003963 |
| cg02320003 | −0.0053547 |
| cg02361878 | 6.81994265 |
| cg02393721 | −0.0020756 |
| cg02434059 | 4.4263351 |
| cg02435538 | −0.0332023 |
| cg02450064 | −0.0044686 |
| cg02486497 | −8.638E−05 |
| cg02491557 | 0.10150297 |
| cg02493740 | −0.0006179 |
| cg02563156 | −0.00027 |
| cg02569613 | −0.0001494 |
| cg02582963 | −0.0007806 |
| cg02653521 | −9.223E−05 |
| cg02691360 | −0.0018682 |
| cg02729030 | −0.0002061 |
| cg02761568 | −0.009326 |
| cg02777885 | −0.0001128 |
| cg02780919 | 2.0397033 |
| cg02827075 | −0.0019161 |
| cg02870946 | −0.0047713 |
| cg02942825 | −0.0129901 |
| cg02954562 | −5.749E−05 |
| cg02965290 | −0.0017316 |
| cg02966722 | −0.0029717 |
| cg02967428 | −0.0001142 |
| cg02995791 | 4.02307911 |
| cg03025337 | −0.0149376 |
| cg03040622 | −0.0015692 |
| cg03077331 | −0.0001188 |
| cg03111404 | 11.2132307 |
| cg03140521 | 3.73788744 |
| cg03155027 | −0.0123522 |
| cg03165014 | −0.0001446 |
| cg03177551 | 1.54347675 |
| cg03186975 | −0.0034034 |
| cg03215416 | 1.80901321 |
| cg03270167 | −0.0011071 |
| cg03277049 | 12.8126008 |
| cg03297163 | −0.000168 |
| cg03310376 | 2.87419955 |
| cg03337277 | −0.0002429 |
| cg03345116 | −0.0018585 |
| cg03405983 | 13.3725261 |
| cg03454541 | −0.0030992 |
| cg03466525 | −5.835E−05 |
| cg03493032 | −0.0024895 |
| cg03507218 | 0.49315792 |
| cg03521737 | −6.482E−05 |
| cg03525069 | −7.545E−05 |
| cg03534847 | 8.48319645 |
| cg03537591 | 8.73685021 |
| cg03554174 | −0.0008098 |
| cg03573179 | −0.0003573 |
| cg03574652 | 4.85122588 |
| cg03588998 | −0.0007444 |
| cg03603381 | −0.0134279 |
| cg03639671 | −0.0286827 |
| cg03641033 | −0.0001127 |
| cg03669147 | −0.0002479 |
| cg03678098 | −5.27E−05 |
| cg03686455 | 7.78826168 |
| cg03688058 | −3.899E−05 |
| cg03739378 | −0.0006963 |
| cg03741653 | −0.0001782 |
| cg03748503 | −0.0216813 |
| cg03755535 | −0.0071813 |
| cg03787711 | −9.557E−05 |
| cg03847705 | −0.0001872 |
| cg03858663 | 3.86639189 |
| cg03864121 | 5.0178558 |
| cg03871460 | −0.017885 |
| cg03887528 | −0.0007793 |
| cg03948781 | −0.0002827 |
| cg03982897 | −0.0007042 |
| cg03995615 | −0.0054975 |
| cg03999130 | −0.0016614 |
| cg04012082 | −0.0001567 |
| cg04013159 | −0.0001653 |
| cg04035728 | −3.333E−05 |
| cg04084236 | −0.0026959 |
| cg04087740 | −0.0020312 |
| cg04115680 | −0.0127436 |
| cg04152629 | −0.0014781 |
| cg04154465 | 2.68227346 |
| cg04194821 | −0.002043 |
| cg04236639 | −9.184E−05 |
| cg04254769 | −0.000109 |
| cg04259907 | −0.0001812 |
| cg04270358 | 5.58002637 |
| cg04292941 | −0.0002322 |
| cg04295372 | −5.812E−05 |
| cg04297819 | −4.461E−05 |
| cg04332818 | 11.2597771 |
| cg04359828 | −4.085E−05 |
| cg04362886 | −0.0002181 |
| cg04365102 | −0.0006964 |
| cg04378886 | −0.0046107 |
| cg04452896 | −0.0002107 |
| cg04531704 | 13.0054582 |
| cg04603184 | −0.0280849 |
| cg04613313 | −0.0058607 |
| cg04654363 | −0.0008042 |
| cg04677158 | −0.000102 |
| cg04682845 | −0.0001174 |
| cg04739880 | −0.0580262 |
| cg04756296 | −0.0009227 |
| cg04760708 | 0.59400494 |
| cg04764624 | −0.0021552 |
| cg04786857 | 5.90217557 |
| cg04872610 | −0.0061115 |
| cg04889790 | −0.001101 |
| cg04897713 | −0.0013779 |
| cg04904276 | −5.389E−05 |
| cg04920452 | −0.0002666 |
| cg04928670 | −0.0005847 |
| cg05001334 | 6.52339754 |
| cg05003422 | 64.7347528 |
| cg05049335 | −0.0001639 |
| cg05056497 | −8.688E−05 |
| cg05059108 | −0.0001181 |
| cg05059607 | 10.1022332 |
| cg05070268 | −0.0004133 |
| cg05081614 | −0.0009276 |
| cg05083128 | −0.0004282 |
| cg05090127 | −0.0019225 |
| cg05090759 | −0.000525 |
| cg05106502 | −0.0301172 |
| cg05131940 | −0.0004133 |
| cg05132222 | −0.0426715 |
| cg05147525 | −0.0002729 |
| cg05156137 | −0.0002524 |
| cg05187965 | −4.318E−05 |
| cg05203213 | −0.000615 |
| cg05208605 | 11.854737 |
| cg05290300 | −5.809E−05 |
| cg05310309 | 2.9479852 |
| cg05323898 | 2.56600548 |
| cg05339588 | −0.003433 |
| cg05374271 | −1E−04 |
| cg05385434 | −3.18E−05 |
| cg05395210 | −0.0073297 |
| cg05399434 | −0.0006185 |
| cg05407338 | −0.0145251 |
| cg05497175 | −0.0002426 |
| cg05507697 | −0.0121701 |
| cg05517697 | 16.1079966 |
| cg05520031 | −0.0001892 |
| cg05523085 | −0.0009532 |
| cg05542681 | 6.69988285 |
| cg05551889 | −0.0017211 |
| cg05561193 | −0.0002567 |
| cg05580441 | 7.8538039 |
| cg05601974 | −0.0004599 |
| cg05630016 | −0.0002043 |
| cg05641529 | 5.81564598 |
| cg05673214 | −0.0029182 |
| cg05709162 | −0.0003372 |
| cg05724110 | 6.43544356 |
| cg05732876 | −0.000465 |
| cg05759421 | 7.48153639 |
| cg05787209 | 0.99914803 |
| cg05800368 | −1.795E−05 |
| cg05850205 | −0.0008792 |
| cg05874888 | −0.0272041 |
| cg05890019 | −0.0001798 |
| cg05903289 | 0.30986479 |
| cg05922911 | 5.88774748 |
| cg05929069 | −0.0020583 |
| cg05951994 | −0.0020771 |
| cg05957749 | −0.0039017 |
| cg05977696 | −0.0851238 |
| cg05985146 | −0.0029178 |
| cg06007201 | −0.0002224 |
| cg06035815 | −0.0009044 |
| cg06116806 | −0.0011006 |
| cg06136160 | −0.0005472 |
| cg06146665 | −0.001896 |
| cg06197751 | −0.0001116 |
| cg06198975 | 3.41309839 |
| cg06270615 | −0.0005422 |
| cg06377473 | −0.0004509 |
| cg06407657 | −4.395E−05 |
| cg06412669 | 0.73105806 |
| cg06418867 | 1.99003237 |
| cg06496666 | −0.0001511 |
| cg06500246 | −8.62E−05 |
| cg06521852 | −0.0001433 |
| cg06559878 | −0.0002516 |
| cg06567829 | 5.61566519 |
| cg06573459 | −0.0001583 |
| cg06599546 | −0.0015005 |
| cg06606003 | 6.1442712 |
| cg06629644 | −0.0002048 |
| cg06636172 | 1.11185354 |
| cg06652313 | 3.30632514 |
| cg06670463 | −0.0153083 |
| cg06675483 | −0.0247562 |
| cg06699216 | 0.09094129 |
| cg06739520 | 64.8969906 |
| cg06806080 | −0.0002464 |
| cg06864533 | −0.0009026 |
| cg06885782 | −0.0002157 |
| cg06908352 | −0.0001194 |
| cg06975196 | −0.0011987 |
| cg06984176 | 6.21451797 |
| cg06994022 | −4.224E−05 |
| cg07003587 | −0.0003245 |
| cg07030727 | −0.0001933 |
| cg07053014 | −0.0068286 |
| cg07068570 | −0.0001402 |
| cg07074042 | −3.471E−05 |
| cg07077694 | −0.0004451 |
| cg07136905 | −0.0006195 |
| cg07142010 | −3.123E−05 |
| cg07186576 | −0.0004449 |
| cg07196577 | −0.000132 |
| cg07203024 | −0.0022773 |
| cg07244098 | −9.885E−05 |
| cg07251046 | 2.71570422 |
| cg07326665 | −0.0008775 |
| cg07330481 | −0.0005948 |
| cg07356549 | −0.0006608 |
| cg07360805 | −0.0021902 |
| cg07364657 | −5.356E−05 |
| cg07420274 | −0.001397 |
| cg07434260 | −0.0022431 |
| cg07435237 | 19.1036172 |
| cg07437373 | −0.0023535 |
| cg07495811 | 2.3897303 |
| cg07561710 | −0.0018393 |
| cg07571531 | 0.4676354 |
| cg07597022 | −0.0002009 |
| cg07598052 | −0.0001623 |
| cg07599979 | −6.439E−05 |
| cg07630301 | 4.39E−05 |
| cg07637837 | −0.0010589 |
| cg07644368 | −0.0001269 |
| cg07657357 | −0.0047499 |
| cg07743805 | −0.004286 |
| cg07751331 | −0.0001395 |
| cg07834249 | 4.53266709 |
| cg07890839 | −0.0030152 |
| cg07907506 | −8.545E−05 |
| cg07924503 | −0.00039 |
| cg07932199 | −0.00143 |
| cg07951355 | −0.0090853 |
| cg07977153 | −0.0005822 |
| cg08017858 | 4.98898635 |
| cg08022502 | −0.0002409 |
| cg08027265 | −0.0015001 |
| cg08084946 | 1.6632373 |
| cg08110542 | −8.5E−05 |
| cg08131204 | 0.64877917 |
| cg08203210 | −0.012207 |
| cg08248751 | −2.204E−05 |
| cg08285446 | −0.0004847 |
| cg08291907 | 0.000357 |
| cg08334034 | −0.0001222 |
| cg08363339 | −8.824E−05 |
| cg08438690 | −0.0013078 |
| cg08480461 | −0.0038183 |
| cg08481354 | 21.123294 |
| cg08516817 | 1.73841036 |
| cg08541155 | −0.0002801 |
| cg08577293 | −0.0062754 |
| cg08591668 | −0.0002832 |
| cg08604223 | −0.0048786 |
| cg08655800 | −0.0009553 |
| cg08661007 | 6.49891285 |
| cg08665251 | −7.234E−05 |
| cg08690999 | −0.0026184 |
| cg08738300 | 2.0055399 |
| cg08759041 | −0.0062415 |
| cg08789022 | 4.33772383 |
| cg08832695 | −0.0035916 |
| cg08841257 | 4.138E−05 |
| cg08897150 | −5.471E−05 |
| cg08915824 | 6.608E−05 |
| cg08939521 | 3.32736941 |
| cg08965235 | −0.005859 |
| cg08980461 | −0.0257429 |
| cg09043511 | −0.0011851 |
| cg09048038 | −9.537E−05 |
| cg09144707 | −0.0004531 |
| cg09173348 | −0.0004748 |
| cg09226596 | 4.5542474 |
| cg09230154 | 7.19588314 |
| cg09236382 | −0.000209 |
| cg09278098 | 0.0015967 |
| cg09293925 | −0.0028738 |
| cg09409484 | −0.0056162 |
| cg09438113 | −9.695E−05 |
| cg09494188 | 11.956544 |
| cg09500200 | −0.0050981 |
| cg09550397 | −0.003231 |
| cg09560549 | −0.0004655 |
| cg09565806 | −0.0014836 |
| cg09582351 | −8.713E−05 |
| cg09584711 | −0.0001069 |
| cg09594075 | −0.0017774 |
| cg09621438 | −0.0034009 |
| cg09650189 | −0.0232144 |
| cg09662798 | −0.0127482 |
| cg09700701 | −0.0052909 |
| cg09729866 | −0.0005413 |
| cg09741713 | −0.024343 |
| cg09754413 | −0.0061506 |
| cg09856367 | 0.0012112 |
| cg09882118 | −0.0093215 |
| cg09904296 | −0.0004129 |
| cg09921821 | −0.0394451 |
| cg09933355 | 4.41143558 |
| cg09978077 | −0.0594639 |
| cg09996325 | −0.0004074 |
| cg10018167 | −0.000496 |
| cg10084554 | −0.0024193 |
| cg10099638 | −0.0040474 |
| cg10101634 | −0.0025154 |
| cg10103850 | −0.0057709 |
| cg10116432 | −0.0003199 |
| cg10129391 | −0.0208828 |
| cg10208370 | −0.0055845 |
| cg10230190 | −0.0005722 |
| cg10240139 | 1.15658674 |
| cg10245988 | 10.4776609 |
| cg10249997 | −0.0008455 |
| cg10258419 | −0.039464 |
| cg10259872 | 0.0001507 |
| cg10324116 | 17.0587639 |
| cg10338112 | −0.017777 |
| cg10339152 | −0.0005657 |
| cg10362475 | 0.66527543 |
| cg10364115 | −7.309E−05 |
| cg10374813 | −0.0047356 |
| cg10395934 | 1.14958131 |
| cg10411590 | −0.0146162 |
| cg10441379 | 0.14473919 |
| cg10461547 | −0.0030334 |
| cg10493259 | −0.0101546 |
| cg10512089 | −7.964E−05 |
| cg10516975 | −0.0007033 |
| cg10536276 | −0.0001087 |
| cg10547057 | −0.007397 |
| cg10577534 | −0.0011148 |
| cg10585486 | −0.0002869 |
| cg10589385 | −0.0020643 |
| cg10601821 | −0.001271 |
| cg10608948 | −0.0001267 |
| cg10619644 | −0.0010473 |
| cg10644544 | −0.0001894 |
| cg10660903 | −7.895E−05 |
| cg10708955 | −0.0002336 |
| cg10722426 | −0.000322 |
| cg10738119 | −0.0335186 |
| cg10745272 | −8.105E−05 |
| cg10762466 | −0.0001697 |
| cg10852096 | −5.116E−05 |
| cg10853431 | 1.23990001 |
| cg10883038 | −0.001899 |
| cg10915716 | −0.0007934 |
| cg10975001 | −0.0033364 |
| cg11004284 | −0.0013133 |
| cg11068337 | −0.0413554 |
| cg11069276 | −0.0014785 |
| cg11083280 | 8.53935096 |
| cg11101109 | 1.35989504 |
| cg11146034 | −0.0005887 |
| cg11146821 | −0.0018657 |
| cg11157584 | 4.89342914 |
| cg11198589 | −0.0022855 |
| cg11209249 | −0.0002196 |
| cg11220565 | −0.0001947 |
| cg11229663 | −0.000407 |
| cg11241549 | 1.76688515 |
| cg11345976 | −2.57E−05 |
| cg11386711 | −3.389E−05 |
| cg11412468 | −0.000133 |
| cg11548083 | −9.298E−05 |
| cg11565786 | 3.15495237 |
| cg11591636 | 3.02592675 |
| cg11619602 | −0.000898 |
| cg11682508 | −8.372E−05 |
| cg11682697 | −0.0203438 |
| cg11754420 | −0.0005461 |
| cg11857646 | −0.0027594 |
| cg11881754 | 25.5170287 |
| cg11898958 | 1.18802409 |
| cg11908751 | −0.0907048 |
| cg11988722 | −0.0023231 |
| cg12023170 | −0.0008938 |
| cg12027899 | 12.3165515 |
| cg12084760 | −0.0003542 |
| cg12183861 | −8.361E−05 |
| cg12193345 | −6.775E−05 |
| cg12198704 | 3.84488304 |
| cg12234855 | 12.308298 |
| cg12236088 | −0.0172554 |
| cg12307333 | −3.134E−05 |
| cg12334488 | 1.62290196 |
| cg12347757 | −0.0167597 |
| cg12369353 | −4.835E−05 |
| cg12419195 | 1.25377797 |
| cg12419863 | −0.0001308 |
| cg12422683 | −0.0001014 |
| cg12435725 | −0.0039013 |
| cg12447832 | −4.735E−05 |
| cg12454167 | −8.495E−05 |
| cg12513221 | −0.0210837 |
| cg12564285 | −0.0003132 |
| cg12565788 | 6.7615952 |
| cg12569592 | 0.0155494 |
| cg12582426 | −0.0002311 |
| cg12614395 | 21.2222197 |
| cg12649238 | −0.0025852 |
| cg12673499 | 5.96889479 |
| cg12676803 | −0.0005171 |
| cg12701088 | −0.0002908 |
| cg12776287 | −0.0007655 |
| cg12781915 | −0.0011424 |
| cg12797879 | 7.11475089 |
| cg12864912 | −0.0002285 |
| cg12903224 | −7.715E−05 |
| cg12904135 | −0.003768 |
| cg12916580 | 0.0002504 |
| cg12968598 | −7.593E−05 |
| cg13007701 | −0.0002422 |
| cg13026730 | −0.001609 |
| cg13121938 | −0.0001486 |
| cg13224583 | 3.65298153 |
| cg13247673 | −0.0010167 |
| cg13257412 | −0.0048973 |
| cg13261390 | 1.20235686 |
| cg13280882 | 2.81217335 |
| cg13296238 | −0.001971 |
| cg13303654 | −0.0007252 |
| cg13331559 | −0.0012942 |
| cg13363708 | −0.0002274 |
| cg13375538 | −0.0004199 |
| cg13432087 | 1.22122453 |
| cg13455704 | −0.003375 |
| cg13456470 | −0.0223684 |
| cg13467814 | −0.033056 |
| cg13483882 | −0.0001351 |
| cg13501538 | −0.0002546 |
| cg13581582 | 3.98482316 |
| cg13601739 | −0.0004669 |
| cg13646005 | −0.0007844 |
| cg13656518 | 21.8594121 |
| cg13665684 | −0.00015 |
| cg13690564 | −0.0014125 |
| cg13695646 | −3.399E−05 |
| cg13711394 | −0.0089063 |
| cg13718185 | −0.0001507 |
| cg13779868 | −0.0001508 |
| cg13793145 | −0.0032572 |
| cg13809095 | −0.0014863 |
| cg13817265 | −0.0003355 |
| cg13826452 | 17.2278413 |
| cg13845561 | −9.585E−05 |
| cg13849525 | −9.496E−05 |
| cg13872005 | −0.0155877 |
| cg13881108 | −0.0414679 |
| cg13882377 | −0.0010323 |
| cg13924974 | −0.0006399 |
| cg13945224 | −0.0486116 |
| cg13952159 | −0.0029825 |
| cg13957413 | −0.0001572 |
| cg13978542 | 8.66716859 |
| cg13983063 | −0.0001113 |
| cg13993274 | −0.0002069 |
| cg14044707 | −0.0003826 |
| cg14072027 | −0.0023965 |
| cg14079463 | 1.35526782 |
| cg14081465 | −0.0012843 |
| cg14088282 | −0.0002709 |
| cg14124917 | −0.0005915 |
| cg14150023 | 4.42920512 |
| cg14186846 | −0.000685 |
| cg14198450 | −0.0001678 |
| cg14202850 | −0.026704 |
| cg14233374 | −0.0184175 |
| cg14242895 | −0.0023335 |
| cg14248680 | −0.0003074 |
| cg14311471 | −0.0010345 |
| cg14329026 | −0.0001717 |
| cg14345882 | −0.0015426 |
| cg14397813 | −0.0008411 |
| cg14409029 | −0.0006048 |
| cg14434109 | −0.0001689 |
| cg14544289 | −0.0003594 |
| cg14570838 | −9.671E−05 |
| cg14611152 | −0.0130718 |
| cg14615833 | 6.32625983 |
| cg14672994 | −0.0009324 |
| cg14682080 | 0.87907021 |
| cg14697880 | −0.0019134 |
| cg14752965 | −0.0017098 |
| cg14781394 | −0.0001298 |
| cg14789828 | −0.000403 |
| cg14907738 | −0.0026835 |
| cg14944923 | −0.0001619 |
| cg14989252 | −0.0002268 |
| cg15072306 | 2.90620111 |
| cg15099418 | −0.0013912 |
| cg15123742 | −0.0027246 |
| cg15149655 | 7.78576433 |
| cg15176213 | −0.0007471 |
| cg15246131 | −0.0016665 |
| cg15298719 | −3.112E−05 |
| cg15308737 | −0.0003995 |
| cg15337006 | −0.0010463 |
| cg15352315 | −0.0002975 |
| cg15461663 | −0.0001275 |
| cg15590153 | −0.0002111 |
| cg15594585 | −0.0003945 |
| cg15681737 | −0.0079353 |
| cg15763258 | −0.000226 |
| cg15770553 | −8.556E−05 |
| cg15787146 | −0.0002709 |
| cg15808924 | −0.000348 |
| cg15841865 | 8.50528102 |
| cg15856275 | −0.0002432 |
| cg15884510 | −0.0004901 |
| cg15890469 | −9.461E−05 |
| cg15907944 | 1.4974289 |
| cg16005271 | 2.2893694 |
| cg16008966 | −0.0082816 |
| cg16038868 | −6.159E−05 |
| cg16043345 | −0.0001471 |
| cg16103959 | 6.35898413 |
| cg16193278 | −0.0118803 |
| cg16206504 | −0.0015043 |
| cg16208357 | 6.06485793 |
| cg16209444 | −0.005406 |
| cg16224163 | −3.449E−05 |
| cg16238336 | −0.0097638 |
| cg16321524 | −0.0073329 |
| cg16404106 | −4.91E−05 |
| cg16408679 | −0.0002478 |
| cg16477774 | −0.0002126 |
| cg16569937 | 0.03433592 |
| cg16578883 | 9.57280333 |
| cg16624069 | −0.0002074 |
| cg16675381 | 7.86339929 |
| cg16699148 | −0.0006365 |
| cg16759221 | −0.0162095 |
| cg16795804 | −1.736E−05 |
| cg16801102 | −0.0683026 |
| cg16844661 | −0.0004073 |
| cg16987524 | 6.20581341 |
| cg17063929 | 7.02311545 |
| cg17076780 | −0.0002294 |
| cg17113968 | −0.0198898 |
| cg17133183 | 1.98373155 |
| cg17207815 | −0.0168735 |
| cg17215446 | −6.714E−05 |
| cg17217665 | −0.0015454 |
| cg17359265 | −4.767E−05 |
| cg17389077 | −3.886E−05 |
| cg17402294 | −0.00011 |
| cg17408380 | 0.0004082 |
| cg17414101 | −8.218E−05 |
| cg17425144 | −5.033E−05 |
| cg17436666 | 1.02165451 |
| cg17479898 | −0.0008005 |
| cg17499941 | −0.0342576 |
| cg17506588 | −0.0004731 |
| cg17526103 | 23.8442804 |
| cg17545662 | −0.0001344 |
| cg17607231 | −0.0007984 |
| cg17701035 | 3.42465059 |
| cg17733447 | −0.0019494 |
| cg17737388 | −0.0003246 |
| cg17745234 | −6.215E−05 |
| cg17768691 | −9.069E−05 |
| cg17774395 | 1.48085437 |
| cg17822955 | −0.0001118 |
| cg17834136 | −0.0005544 |
| cg17841545 | −0.0026052 |
| cg17880593 | −0.0001726 |
| cg17956485 | −0.0024072 |
| cg18044113 | 2.652512 |
| cg18045859 | 1.18082014 |
| cg18064468 | −0.0944369 |
| cg18087520 | −0.0001246 |
| cg18116154 | −6.638E−05 |
| cg18180155 | 2.57701971 |
| cg18183774 | −0.0003991 |
| cg18247172 | −0.0002149 |
| cg18267943 | −0.0002648 |
| cg18282455 | −0.0001063 |
| cg18282791 | 1.30763486 |
| cg18321051 | −0.0003861 |
| cg18327056 | 43.5868352 |
| cg18401778 | −6.828E−05 |
| cg18427905 | −0.0005622 |
| cg18544564 | −0.0009755 |
| cg18559348 | −0.0055978 |
| cg18585107 | −0.0001125 |
| cg18618230 | 0.69050983 |
| cg18626035 | −0.0001994 |
| cg18653195 | −0.0003682 |
| cg18664965 | −3.764E−05 |
| cg18675610 | −5.964E−05 |
| cg18742814 | −0.0005056 |
| cg18750833 | −9.468E−05 |
| cg18770216 | −0.0012642 |
| cg18815343 | 0.05105114 |
| cg18825119 | −0.000669 |
| cg18954925 | 0.23873446 |
| cg19003183 | −0.0159285 |
| cg19009417 | 9.54249755 |
| cg19040266 | −0.0009853 |
| cg19065106 | −0.0001724 |
| cg19091930 | −0.0098259 |
| cg19115882 | 0.00208883 |
| cg19120897 | −0.00056 |
| cg19162496 | 0.0005801 |
| cg19220282 | −7.129E−05 |
| cg19261426 | −0.0020876 |
| cg19306368 | −0.0002913 |
| cg19308769 | −0.0001318 |
| cg19395684 | −0.0001861 |
| cg19399220 | 8.61955651 |
| cg19413366 | −0.0001143 |
| cg19430473 | −4.079E−05 |
| cg19437258 | −0.0072275 |
| cg19490598 | 10.2505587 |
| cg19537511 | −0.0253125 |
| cg19630374 | 3.23212533 |
| cg19636519 | −3.49E−05 |
| cg19704902 | −0.0090227 |
| cg19735514 | −6.431E−05 |
| cg19764489 | 0.62810655 |
| cg19766591 | −0.0012732 |
| cg19776272 | −0.0013346 |
| cg19782411 | −0.0031379 |
| cg19801062 | −0.0067036 |
| cg19866195 | −0.0202711 |
| cg20116199 | 1.1357039 |
| cg20124188 | −0.0010901 |
| cg20140333 | −0.00017 |
| cg20169823 | −0.0001982 |
| cg20185615 | −0.0002476 |
| cg20224218 | −0.0003727 |
| cg20250935 | −0.0001614 |
| cg20359042 | 17.1283852 |
| cg20364660 | −4.241E−05 |
| cg20375093 | −0.000163 |
| cg20401567 | −3.169E−05 |
| cg20471783 | −0.0009638 |
| cg20481287 | −0.0001435 |
| cg20485144 | −0.0008893 |
| cg20630655 | −0.0001081 |
| cg20651988 | −0.0071973 |
| cg20651995 | −0.0006424 |
| cg20668177 | −0.0062858 |
| cg20671920 | −0.0224186 |
| cg20720686 | −0.0493596 |
| cg20811236 | −0.0002026 |
| cg20885815 | −0.0125034 |
| cg20891481 | −0.0185041 |
| cg20918218 | −0.0130935 |
| cg20918537 | −0.0029454 |
| cg20933634 | −1.958E−05 |
| cg20957523 | −0.0013369 |
| cg20959174 | −0.0140911 |
| cg20981127 | −0.0001326 |
| cg21075829 | −0.0139925 |
| cg21093005 | 6.10425884 |
| cg21108553 | 15.3049011 |
| cg21121119 | −0.0052173 |
| cg21123203 | −0.0038214 |
| cg21139935 | 0.76268371 |
| cg21154793 | −0.0006865 |
| cg21161126 | −0.0002536 |
| cg21220286 | −0.0018809 |
| cg21234538 | −0.0133544 |
| cg21243944 | −0.0001386 |
| cg21250433 | −0.0009878 |
| cg21274570 | 5.24734363 |
| cg21322248 | −0.0002643 |
| cg21323720 | −0.0010616 |
| cg21356710 | −0.0002582 |
| cg21415385 | −0.0009808 |
| cg21461308 | 1.64365821 |
| cg21467697 | 0.18652956 |
| cg21496419 | −2.932E−05 |
| cg21602257 | −0.0002784 |
| cg21635854 | 8.90734171 |
| cg21639287 | −0.000108 |
| cg21649013 | −0.0027407 |
| cg21672550 | −0.007254 |
| cg21684021 | −0.0067514 |
| cg21689228 | 4.45839418 |
| cg21709322 | −0.006264 |
| cg21737698 | 0.0057604 |
| cg21748751 | −0.0062904 |
| cg21761922 | −7.216E−05 |
| cg21801165 | −0.0017119 |
| cg21807240 | −0.0009188 |
| cg21863334 | −0.002387 |
| cg21886042 | −0.0046166 |
| cg22013564 | −0.0001408 |
| cg22017213 | −0.0001402 |
| cg22021934 | −0.0019246 |
| cg22082347 | −0.0004049 |
| cg22158854 | −4.854E−05 |
| cg22160472 | −0.0005102 |
| cg22217449 | 18.5309187 |
| cg22271663 | 5.61557194 |
| cg22277154 | −0.0006998 |
| cg22326328 | −0.0008419 |
| cg22360016 | −0.0135373 |
| cg22378853 | −0.0014666 |
| cg22508957 | −3.569E−05 |
| cg22685966 | 5.37979139 |
| cg22695707 | 0.03456224 |
| cg22708914 | −0.0002942 |
| cg22711218 | −0.0018067 |
| cg22714777 | −0.0035996 |
| cg22801992 | −0.0001165 |
| cg22853588 | −0.0003977 |
| cg22917652 | −7.419E−05 |
| cg22927247 | −0.0019981 |
| cg22968401 | −0.0002374 |
| cg22987011 | −4.724E−05 |
| cg23003720 | −0.0075371 |
| cg23012731 | 0.0035634 |
| cg23075364 | −0.0034308 |
| cg23090606 | −0.0002289 |
| cg23112821 | −0.0003224 |
| cg23251687 | 0.0779886 |
| cg23273694 | −0.0021948 |
| cg23275972 | −0.0003978 |
| cg23305365 | −0.0002609 |
| cg23307858 | −5.12E−05 |
| cg23322112 | −0.0019918 |
| cg23333513 | 6.06323566 |
| cg23361092 | 28.3906528 |
| cg23371476 | −0.0014064 |
| cg23435915 | −8.616E−05 |
| cg23445859 | 5.30497787 |
| cg23452458 | −0.0022929 |
| cg23456221 | −0.0006602 |
| cg23463186 | 4.389869 |
| cg23469025 | −0.0035193 |
| cg23550082 | −0.0075443 |
| cg23674344 | −0.0005598 |
| cg23687466 | −0.0004851 |
| cg23694533 | −0.0008531 |
| cg23703060 | −0.0001056 |
| cg23752007 | −2.122E−05 |
| cg23765126 | −0.00527 |
| cg23817643 | 11.8252186 |
| cg23831735 | −0.0140736 |
| cg23939642 | 1.51259668 |
| cg24012925 | −3.868E−05 |
| cg24023487 | −0.0001521 |
| cg24030449 | −0.0004375 |
| cg24053748 | 8.21800989 |
| cg24057642 | −0.0001166 |
| cg24070213 | −6.375E−05 |
| cg24117274 | −5.35E−05 |
| cg24129115 | −0.0029211 |
| cg24158141 | 4.56957546 |
| cg24159575 | −0.0098242 |
| cg24217726 | −0.0028829 |
| cg24251135 | −0.0002752 |
| cg24254377 | −0.0001235 |
| cg24284497 | −0.0004729 |
| cg24315876 | −0.007627 |
| cg24322968 | −0.0050598 |
| cg24387126 | −4.558E−05 |
| cg24399540 | 0.45394521 |
| cg24415799 | −0.0004079 |
| cg24442609 | −0.0007599 |
| cg24475171 | −0.0001576 |
| cg24496614 | −0.0238293 |
| cg24514884 | −0.0002707 |
| cg24609819 | −0.0005618 |
| cg24636368 | −0.0033239 |
| cg24637417 | −0.0003536 |
| cg24642820 | −0.0001463 |
| cg24686551 | −0.0632836 |
| cg24710309 | 7.03287791 |
| cg24727203 | −0.0002095 |
| cg24733384 | −0.0002479 |
| cg24741744 | −0.0013545 |
| cg24749947 | −0.0005326 |
| cg24761567 | −0.0003084 |
| cg24765360 | −0.0013858 |
| cg24787755 | −0.0014955 |
| cg24815934 | −0.0001675 |
| cg24830730 | −0.0047783 |
| cg24856658 | −0.0001345 |
| cg24891133 | 9.78349506 |
| cg24928110 | −0.0012956 |
| cg24947451 | −0.0001493 |
| cg24950222 | −0.0011538 |
| cg24987259 | −0.0003173 |
| cg25064552 | 0.8056747 |
| cg25164649 | −0.0522758 |
| cg25284762 | −0.0039331 |
| cg25352397 | 0.64203147 |
| cg25359664 | −0.0003261 |
| cg25428553 | −0.0132 |
| cg25557995 | 3.2690613 |
| cg25615068 | −0.0239951 |
| cg25618559 | 7.09091958 |
| cg25671484 | −5.211E−05 |
| cg25673241 | 13.5426927 |
| cg25741837 | −0.0025648 |
| cg25744957 | −0.0003404 |
| cg25814293 | −0.0095645 |
| cg25815229 | −0.0075277 |
| cg25822709 | −0.0001013 |
| cg25848076 | −0.0012687 |
| cg25875213 | −0.0447445 |
| cg25904183 | −0.0001499 |
| cg25912009 | −0.0011492 |
| cg25979108 | −0.008859 |
| cg25982743 | −0.0002782 |
| cg26004707 | −0.0149953 |
| cg26012941 | −0.0013209 |
| cg26034375 | −8.728E−05 |
| cg26035489 | −0.0005682 |
| cg26058502 | −0.0002542 |
| cg26084258 | −0.00814 |
| cg26109145 | −0.0100455 |
| cg26124115 | −0.0029991 |
| cg26127836 | −0.0013067 |
| cg26128121 | −0.0011639 |
| cg26128464 | 1.3918397 |
| cg26134777 | −0.0022724 |
| cg26137103 | −9.524E−05 |
| cg26149658 | −0.0001092 |
| cg26248173 | −0.0018164 |
| cg26250086 | −0.0001193 |
| cg26260789 | −6.808E−05 |
| cg26261298 | −0.0011119 |
| cg26276947 | 1.86575949 |
| cg26282505 | −1.288E−05 |
| cg26287345 | −0.0005207 |
| cg26343958 | −8.329E−05 |
| cg26425555 | −1.488E−05 |
| cg26439710 | −0.0074881 |
| cg26564280 | −0.0011225 |
| cg26572392 | −0.0005166 |
| cg26578149 | −0.0001731 |
| cg26644052 | −0.0006888 |
| cg26680047 | 4.52886414 |
| cg26684319 | −2.705E−05 |
| cg26692296 | 6.00575743 |
| cg26692822 | −0.0007135 |
| cg26749414 | 1.17194167 |
| cg26767214 | −0.0031972 |
| cg26782013 | −3.483E−05 |
| cg26784012 | −0.0001495 |
| cg26815396 | −0.0019934 |
| cg26843498 | −3.808E−05 |
| cg26863750 | −0.0040026 |
| cg26875135 | −8.449E−05 |
| cg26884773 | −0.0006079 |
| cg26901111 | −0.0005769 |
| cg26916966 | −0.0860738 |
| cg26940479 | −0.0011618 |
| cg26951440 | −8.971E−05 |
| cg26971710 | −0.0070676 |
| cg27000590 | −0.000167 |
| cg27004870 | −0.0030244 |
| cg27089226 | −0.000235 |
| cg27130359 | −0.0012633 |
| cg27134322 | 2.20380511 |
| cg27139956 | −0.0001189 |
| cg27144223 | −8.596E−05 |
| cg27184585 | −0.003216 |
| cg27189341 | −4.542E−05 |
| cg27189533 | −0.0006776 |
| cg27208169 | −0.0003438 |
| cg27222157 | 3.1391154 |
| cg27292417 | 1.70093743 |
| cg27304328 | 4.05536228 |
| cg27342333 | −0.0302718 |
| cg27346545 | −0.0012903 |
| cg27355006 | −0.0074673 |
| cg27368025 | −9.058E−05 |
| cg27379915 | −0.0011042 |
| cg27413008 | −8.407E−05 |
| cg27587195 | 61.1972799 |
| cg27598107 | −0.0005066 |
| cg27598956 | −0.0021883 |
| cg27615366 | −0.0007188 |
| cg27631597 | −0.0037344 |
| cg27660099 | 2.94705724 |
| cg27661460 | −6.912E−05 |
| ch.1.237398078F | −0.0026718 |

### EXAMPLES

The invention is further described in the following examples, which do not limit the scope of the invention described in the claims.

### Methods

The following materials and methods were used in the Examples set forth herein.

### Framingham Heart Study Cohort

The FHS cohort 1 is a large-scale longitudinal study started in 1948, initially investigating the common factors of characteristics that contribute to cardiovascular disease (CVD), framinghamheartstudy.org/index.php. The study initially enrolled participants living in the town of Framingham, Massachusetts, who were free of overt symptoms of CVD, heart attack or stroke at enrollment. In 1971, the study started the FHS Offspring Cohort to enroll a second generation of the original participants' adult children and their spouses (n=5124) to conduct similar examinations. Participants from the FHS Offspring Cohort were eligible for our study if they attended both the eighth examination cycle and consented to having their molecular data used for the study. We used 2,544 participants from the group of Health/Medical/Biomedical (IRB, MDS) consent with available DNA methylation array data. The FHS data are available in dbGaP (accession number: phs000363.v16.p10 and phs000724.v2.p9).

Deaths among the FHS participants that occurred prior to Jan. 1, 2013 were ascertained using multiple strategies, including routine contact with participants for health history updates, surveillance at the local hospital and in obituaries of the local newspaper, and queries to the National Death Index. Death certificates, hospital and nursing home records prior to death, and autopsy reports were requested. When cause of death was undeterminable, the next of kin were interviewed. The date and cause of death were reviewed by an endpoint panel of 3 investigators. Peripheral blood samples were collected at the 8th examination. Genomic DNA was extracted from buffy coat using the Gentra Puregene DNA extraction kit (Qiagen) and bisulfite converted using the EZ DNA Methylation kit (Zymo Research Corporation). DNA methylation quantification was conducted in two laboratory batches using the Illumina Infinium HumanMethylation450 array (Illumina). Methylation beta values were generated using the Bioconductor minfi package with Noob background correction.

### Women's Health Initiative

The WHI is a national study that enrolled postmenopausal women aged 50-79 years into the clinical trials (CT) or observational study (OS) cohorts between 1993 and 19986.7. We included 2107 WHI participants with available phenotype and DNA methylation array from “Broad Agency Award 23” (WHI BA23). WHI BA23 focuses on identifying miRNA and genomic biomarkers of coronary heart disease (CHD), integrating the biomarkers into diagnostic and prognostic predictors of CHD and other related phenotypes. The death status was based on the variable DEATHALL (All Discovered Death) as listed in the form “All Discovered Death Outcome Detail (Form 124/120)”, generated on Mar. 1, 2017. This variable does not censor deaths that occur after the participants' last consent period. The original WHI study began in the early 1990s and concluded in 2005. Since 2005, the WHI has continued as Extension Studies (Ext1), which are annual collections of health updates and outcomes in active participants. The second Extension Study (Ext2) enrolled 93,500 women in 2010 and follow-up of these women continues annually. Death was adjudicated for clinical trial (CT) and observational study (OS) participants through Ext1. In Ext2, death is only adjudicated for the Medical Record Cohort (MRC). Non-MRC cause of death is determined by the initial cause of death form (form 120).

### DNA Methylation Quantification

In brief, bisulfite conversion using the Zymo EZ DNA Methylation Kit (Zymo Research, Orange, CA, USA) as well as subsequent hybridization of the HumanMethylation450k Bead Chip (Illumina, San Diego, CA), and scanning (iScan, Illumina) were performed according to the manufacturers protocols by applying standard settings. DNA methylation levels (β values) were determined by calculating the ratio of intensities between methylated (signal A) and un-methylated (signal B) sites. Specifically, the β value was calculated from the intensity of the methylated (M corresponding to signal A) and un-methylated (U corresponding to signal B) sites, as the ratio of fluorescent signals β=Max (M,0)/[Max (M,0)+Max (U,0)+100]. Thus, β values range from 0 (completely un-methylated) to 1 (completely methylated).

DNA Methylation Quantitative Trait Loci (meQTLs)

cis-meQTLs used in the study were obtained from the Genetics of DNA Methylation Consortium (GoDMC). DNA methylation levels were measured in whole blood samples from 36 cohorts, including 27,750 European subjects. In total, 420,509 CpG sites were analyzed to map the genetic influences on DNA methylation levels. The cis-acting meQTLs were defined as meQTLs within 2 MB window around the target CpG site. GoDMC summary statistics are available at mqtldb.godmc.org.uk.

### GWAS Data for DNA Methylation and Aging-Related Phenotypes

The twelve aging-related traits examined in the study include two lifespan-related traits (lifespan and extreme longevity)^{48,49}, three health-related traits (healthspan, frailty index, and self-rated health)^{50,51}, four epigenetic age measurements (Horvath age, Hannum age, PhenoAge, and GrimAge)⁵², and three summary-level aging-related traits (Aging-GIP1, adjusted Aging-GIP1, and healthy aging)^{53.54}.

For the two traits related to lifespan, the parental lifespan GWAS included a total of 512,047 mothers and 500,193 fathers of European ancestry. For GWAS, the parental lifespan was equivalent to the lifespan of individuals, since the genetic effect on a parental phenotype is expected to be half of the individual's phenotype itself⁴⁸. The extreme longevity GWAS included 11,262 subjects of European ancestry with a lifespan above the 90th percentile as the case group and 25,483 control subjects whose age at the last visit was below the 60th percentile age⁴⁹.

For the three health-related traits, healthspan was defined as the age of the first incidence of any major age-related disease, including dementia, congestive heart failure, diabetes, chronic obstructive pulmonary disease, stroke, cancer, myocardial infarction, as well as the incidence of death⁵⁰. The GWAS of healthspan included 300,447 subjects of European ancestry from the UK Biobank cohort, aged 37 to 73. The frailty index was calculated based on the cumulative number of health deficits during aging⁵¹. The frailty index GWAS included 164,610 UK Biobank participants aged 60-70 years and 10,616 Swedish TwinGene participants aged 41-87 years⁵⁵. Self-rated health GWAS was based on questionnaire responses on a scale of 0-5 in the UK Biobank cohort.

For the four epigenetic age measurements, the epigenetic age was based on various aging clock models, including Horvath age (353 CpG sites), Hannum age (71 CpG sites), PhenoAge (513 CpG sites), and GrimAge (1,030 CpG sites), are calculated in 34,710 participants of European ancestry⁵². All summary statistics of GWAS are publicly available.

For the three summary-level trait, the Aging-GIP1 is the first genetic principle component of six human aging traits-healthspan, father and mother lifespan, exceptional longevity, frailty index and self-rated health, which captures both length of life and indices of mental and physical wellbeing⁵³. The Aging-GIP1-adj is the aging-GIP1 adjusted for household income and socioeconomic deprivation. The Healthy Aging is the meta-analysis of healthspan, lifespan, and longevity⁵⁴.

### Genetic Correlation Analysis

Genetic correlation between traits related to aging is calculated using the LD score regression (LDSC)⁵⁶. SNPs that were imperfectly imputed with INFO less than 0.9 or with a low minor allele frequency less than 5% were removed to reduce statistical noise. LDSC was performed using LDSC software v1.0.1 (github.com/bulik/ldsc).

### Epigenome-Wide Mendelian Randomization Analysis

In MR analysis, the definition of causal relationship is that associations of SNPs with CpG methylation are directionally consistent and proportional in magnitude to associations of SNPs with aging-related phenotypes. Genetic variants that are strongly associated with whole blood DNA methylation level (FDR<0.05) were used for the MR analysis. Only meQTLs in the cis-acting regions were used to avoid pleiotropic effects. As the generalized MR method achieves a higher statistical power by including partially correlated instruments while accounting for the LD structure, we used LD clumping to only remove meQTLs with strong LD (r²>0.3), as suggested by Burgess et al.³⁴. Three MR methods were used for the main analyses: Wald ratio when only one meQTL was available, generalized inverse variance weighted (gIVW) when at least two meQTLs were available, and generalized MR-Egger regression (gEgger) when at least three meQTLs were available^{34,57,58}. The MR analyses were conducted using the MendelianRandomization R package and TwoSampleMR R package (github.com/MRCIEU/TwoSampleMR)^{59.60}.

We only included cis-meQTLs (meQTLs located within 2 MB of target CpG sites) in our analysis to avoid pleiotropic effects, as they are more likely to affect DNA methylation via direct mechanisms. To remove additional pleiotropic effects, we used the results of gEgger, whose estimate is robust to directional pleiotropic effects if the significant intercept is detected by gEgger regression (P<0.05).

CpG-phenotype pairs with P_{adjusted}<0.05 after Bonferroni correction were used to select causal CpG sites with the strongest MR evidence. All CpG-phenotype pairs with FDR <0.05 were considered potential causal CpG sites and used in the downstream sensitivity analysis.

### Sensitivity Analyses

### Horizontal Pleiotropy.

The horizontal pleiotropic effect in instrumental variants may cause biased causal effect estimation from the gIVW method. To detect unbalanced horizontal pleiotropy among genetic instruments, we used the intercept gEgger regression, which provides an estimate of the directional pleiotropic effect⁶¹ Note that by including a partially correlated instrument, the gEgger intercept also has more statistical power to detect the pleiotropic effect. CpG-phenotype pairs with gEgger intercept P value less than 0.05 were potentially affected by the pleiotropic effect, and the gIVW method may be biased. We, therefore, reported an estimate and P value from the gEgger method instead of the gIVW method for these MR signals, as the gEgger estimate is robust to the horizontal pleiotropic effect⁶¹.

### Heterogeneity.

To detect heterogeneity of the MR estimates in each meQTL, we performed the Cochran's Q test and the Rücker's Q test for the gIVW and gEgger results, respectively. Since heterogeneity does not necessarily affect causal effect estimation, we kept the MR signals heterogeneous while reporting potential heterogeneity in the result table.

### Directionality Test.

To exclude MR signals caused by reverse causality (i.e., methylation changes caused by outcome phenotype), we applied MR Steiger test⁶², which is the method to test the directionality of causal effect estimated by MR. We then removed all MR signals with reverse directionality.

### Colocalization Analysis.

Genetic colocalization is a Bayesian approach that estimates the probability (PP.H4) of overlapping genetic signals between molecular traits and outcome is due to both traits sharing a causal variant⁶³. It is an important method to control false positive results from MR and filter out the MR signals purely driven by LD or pleiotropy. All MR signals that passed the FDR threshold of 0.05 were then subjected to the colocalization analysis. We applied pairwise a conditional and colocalization (PWCoCo) analysis⁶⁴, which is a powerful genetic colocalization approach that is able to detect multiple independent genetic signals. We considered colocalization probability (PP.H4)>70% as strong evidence of colocalization. Also, since aging-related GWAS are in general noisy while cis-meQTL usually have strong genetic signals, colocalization probability tends to be low, and the probability of only having a signal from meQTLs (PP.H1) tends to be high. To overcome bias due to imbalanced power between exposure and outcome traits, we considered a conditional colocalization probability (conditional PP.H4=PP.H4/PP.H3+H4) by assuming that the aging-related trait always has genetic signals in the region when a significant MR signal is detected. We then also reported CpG-phenotype pairs with conditional PP.H4 >70% as a potentially colocalized signal.

### Mediation Analysis

We conducted multivariable MR (MVMR) to dissect significant CpG-phenotype causal effects (θ_{T}) into direct effects (θ_{D}) and indirect effects through transcript levels following the methodology outlined in Sadler et al., 2022 and using the smr-ivw software (github.com/masadler/smrivw)⁶⁵. Genetic effect sizes on CpGs (mQTLs) came from the GoDMC consortium (N=32,851)⁴⁵, and on transcript levels (eQTLs) from the eQTLGen consortium (N=$31,684)²¹, both derived from whole blood. Mediation analyses were assessed for CpG-Aging-GIP1 and CpG-adjusted Aging-GIP1 pairs.

Transcript mediators were selected to be in cis (<500 kb away from the CpG site) and causally associated to the CpG. This latter condition was satisfied when univariable MR effects from the CpG site on the transcript had an MR p-value<0.01. Instrumental variants were required to be associated to either the CpG or included transcripts and as in the univariable MR analysis they were selected to be correlated at r²≤0.3. The mediation proportion (MP) was estimated as 1−\hat{θ}_{D}/\hat{θ}_{T}.

### Causality-Informed Epigenetic Clock Model

Elastic net regression is a regularized linear model that solves the problem.

$$\hat{β} = arg min ( RSS ( β ) + λ J ( β ) )$$

where RSS(β) is the residual sum of squares, λ is the regularization parameter, and J(β) is the regularization term⁶⁶. The J(β) term is the sum of the L1 and L2 terms, which is defined as

$$J ( β ) = α {\sum }_{f = 1}^{p} {w}_{f} \vert {β}_{f} \vert + \frac{1}{2} ( 1 - α ) {\sum }_{f = 1}^{p} {w}_{f} {β}_{f}^{2}$$

The parameter is the elastic net mixing parameter, which controls the balance between the L1 and L2 terms. w_{f} is the penalty factor for each feature we introduced. In a regular epigenetic clock model, w_{f} is defined to be 1 for all the features, which produces the model that is purely based on correlation^{67,68}. We introduced a causality-informed elastic net model, where we defined the feature-specific penalty factor as

$${w}_{f} = \frac{p {s}_{f}}{{\sum }_{f = 1}^{p} {s}_{f}}$$

$${s}_{f} = {( max ( {c}_{f} ) + min ( {c}_{f} ) - {c}_{f} )}^{τ}$$

Here the c_{f} is the absolute value of the causality score for each feature, which is calculated from the causal effect size from MR weighted by colocalization probability. The τ>0 is a tuneable parameter that controls how much the causality score affects the feature-specific penalty factor. If τ=0, the whole model is reduced to a regular elastic net regression, where w_{f}=1 for all the features. When t becomes large, the model is more influenced by the causality score and tends to assign larger coefficients to the features with a higher causality score. To balance the precision and causality, we defined τ as 0.3, which is the largest τ value with MAE<5 years in the validation set and maximized the association with mortality.

Using this method, we trained the model on whole-blood methylation data from 2,664 individuals³⁵. We built the causality-informed epigenetic clock model CausAge (See Table A). To separately measure adaptive and damaging DNA methylation changes during aging, we further separated the causal CpG sites into two groups based on causal effect size from MR and the direction of age-related changes. We then built DamAge, a damaging clock (see Table B), and AdaptAge, a protective clock (see Table C).

### Mortality Association Analysis

To evaluate our novel causal clocks for predicting all-cause mortality risk, we applied the clocks to a large-scale dataset comprising 4,651 individuals from (a) the Framingham Heart Study, FHS offspring cohort (n=2,544 Caucasians, 54% females)^{48,49} and (b) Women's Health Initiative cohort^{50,51} (WHI, n=2107 postmenopausal women). Methylation levels were profiled in blood samples based on Illumina 450k arrays. In FHS, the mean (SD) chronological age at the time of the blood draw was 66.3 (8.9) years old. During follow-up, 330 individuals died. The mean (SD) follow-up time (used for assessing time-to-death due to all-cause mortality) was 7.8 (1.7) years. The WHI cohort is a national study that enrolled postmenopausal women aged 50-79 years. Our WHI data consists of three ethnic/racial groups: 47% European ancestry (Caucasians), 32% African Americans, and 20% Hispanic ancestry. All the three ethnic groups have marginally the same age distribution, with a mean (SD) of 65.4 (7.1) years old. The mean (SD) of follow-up time was 16.9 (4.6) years. During follow-up, 765 women died. To evaluate our clocks, we first defined age acceleration (AgeAccel) measure using the residuals resulting from regressing the DNAm variable on chronological age. As noted, this AgeAccel measure is independent of chronological age. Next, we applied Cox regression analysis for time-to-death (as a dependent variable) to assess the predictive ability of our causal clocks for all-cause mortality, using the AgeAccel measures. The analysis was adjusted for age at the blood draw and adjusted for gender and batch effect in FHS. We stratified the WHI cohort by ethnic/racial groups and combined a total of 4 results across FHS and WHI cohorts by fixed effect models weighted by inverse variance. The meta-analysis was performed in the R metafor function.

### Example 1. Epigenome-Wide Mendelian Randomization on Aging-Related Phenotypes

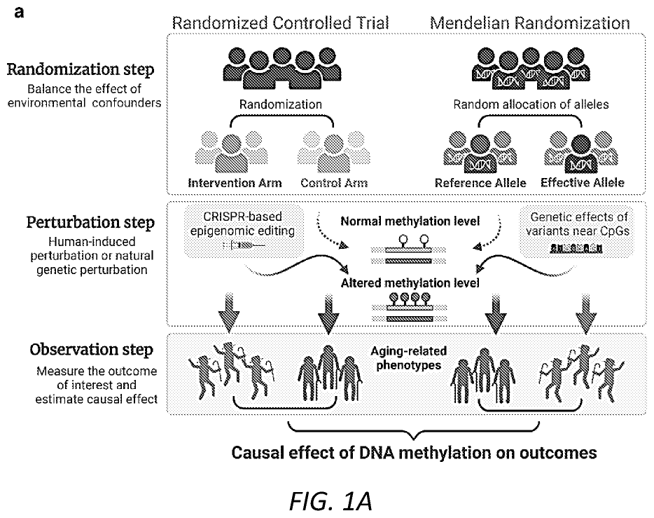

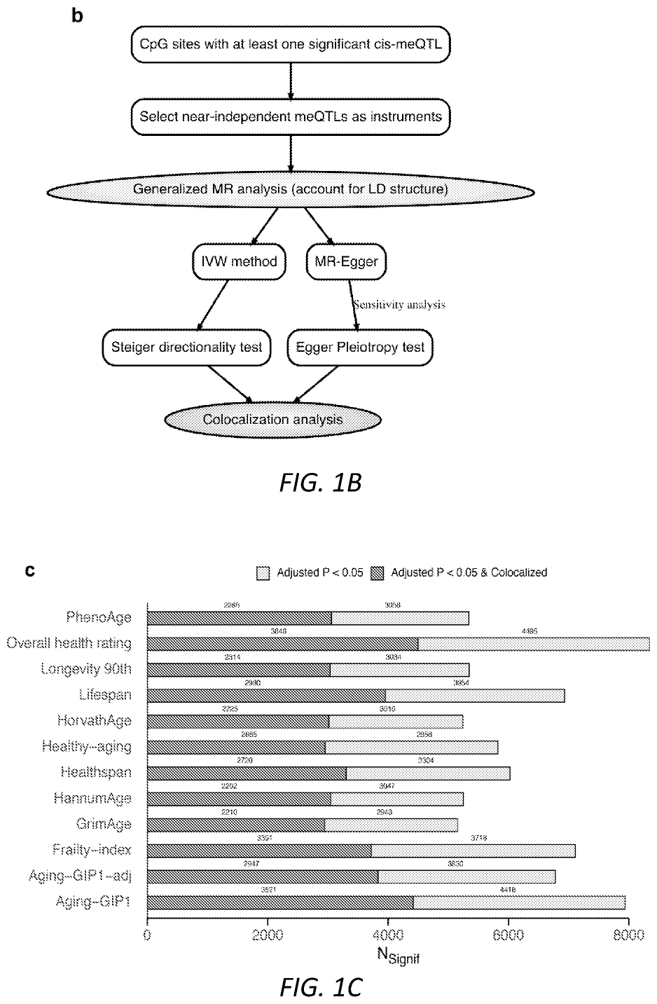

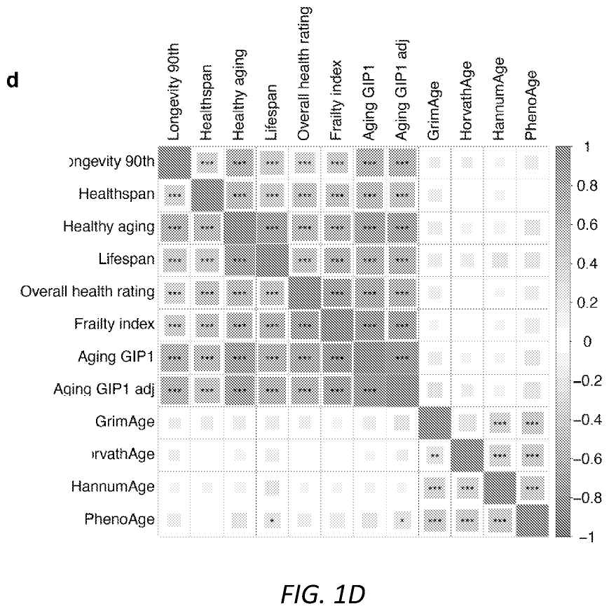

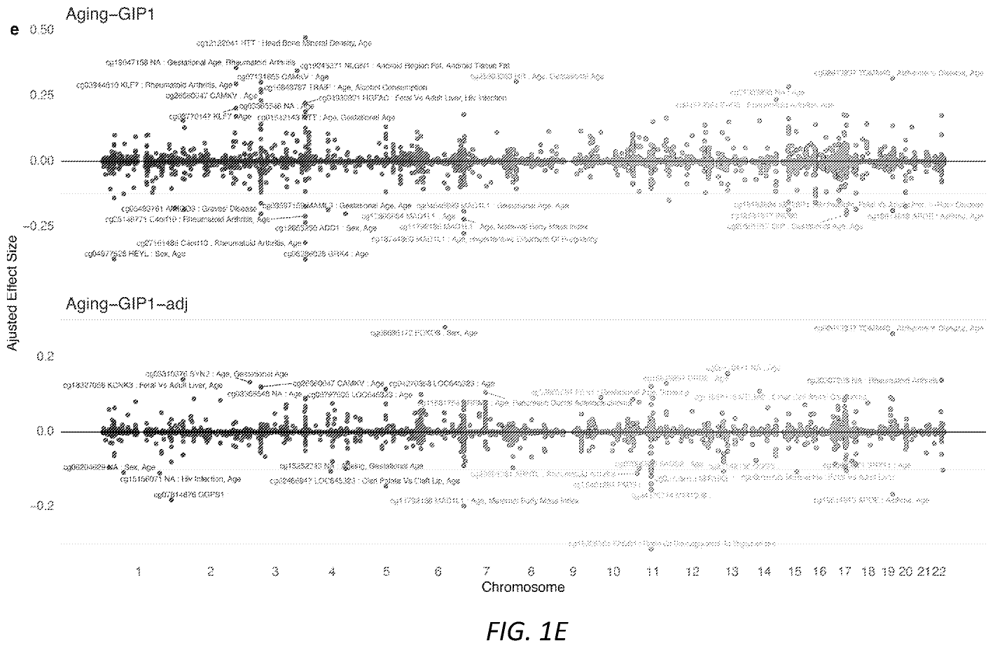

MR is an established genetic approach for causal inference that utilizes natural genetic variants as instrument variables. Since the allocation of genetic variants is a random process and is determined during conception, the causal effects estimated using MR are not biased by environmental confounders. Therefore, it could be used as a tool for investigating causal relationships between the DNA methylation and aging-related phenotypes (FIG. 1a). In the context of MR, a CpG site can be defined as causal when associations of SNPs with CpG methylation are directionally consistent and proportional in magnitude to its associations with aging-related phenotypes.

To identify CpG sites causal to aging, we used 420,509 CpG sites with meQTLs available (GoDMC, whole blood samples from 36 cohorts, 27,750 European subjects) as exposures and selected twelve aging-related phenotypes as outcomes (FIG. 1a, Methods, Table 1), including two lifespan-related traits: lifespan and extreme longevity (defined as survival above 90th percentile)⁵²; three health-related traits: healthspan (age at the first incidence of any major age-related disease), frailty index (measurement of frailty based on the accumulation of a number of health deficits during the life course), and self-rated health (based on the questionnaire responses)^{53,54}; four epigenetic age measurements (Horvath age, Hannum age, PhenoAge, and GrimAge)⁵³; and three summary-level aging-related traits: Aging-GIP1 (the first genetic principle component of six human aging traits-healthspan, father's and mother's lifespan, exceptional longevity, frailty index and self-rated health), socioeconomic traits-adjusted Aging-GIP1, and healthy aging (multivariate genomic scan of healthspan, lifespan, and longevity)⁵³.

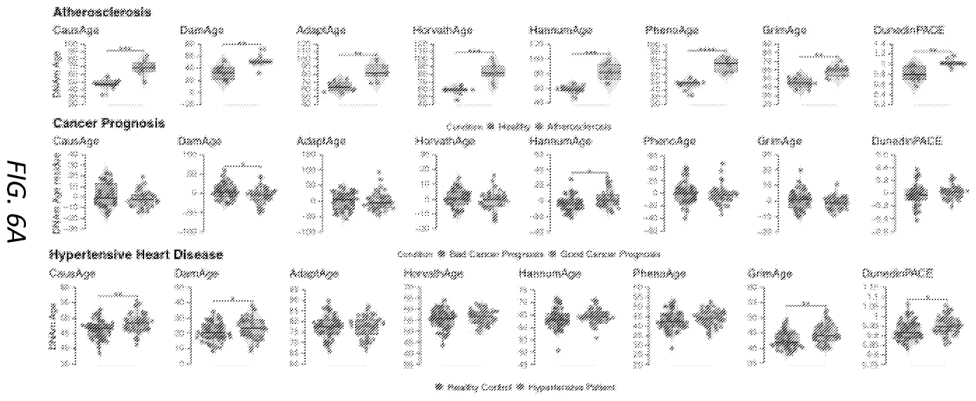

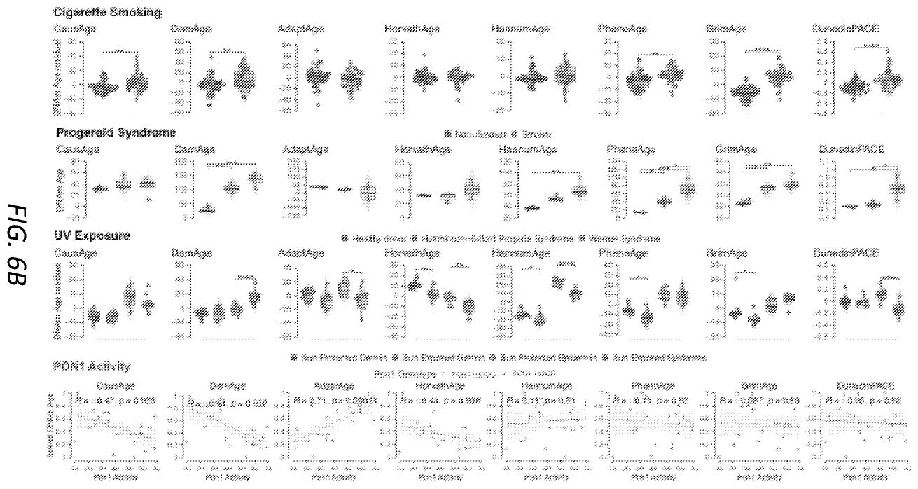

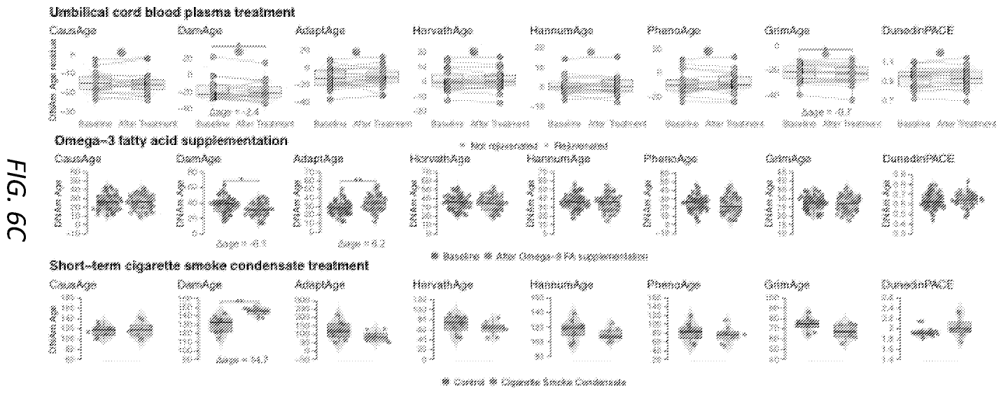

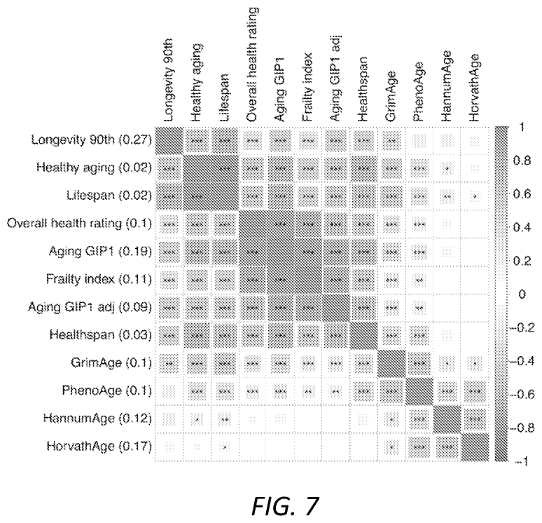

Aging-GIP1 captures both the length of life and age-related health status⁶⁹, which can be considered as a genetic representation of healthy longevity. It also shows the strongest genetic correlation with all other traits related to lifespan⁵³. Therefore, we further used Aging-GIP1 as the primary aging-related trait to investigate CpG sites causal to the aging process. A genetic correlation analysis showed that all eight lifespan- and health-related traits are genetically correlated and clustered with each other, while the four epigenetic age measurements clustered with each other. GrimAge and PhenoAge showed significant genetic correlations with other health and lifespan-related traits, while Hannum age and Horvath age did not (FIG. 7).

We then applied generalized inverse-variance weighted MR (gIVW) and MR-Egger (gEgger) on each exposure-outcome pair (FIG. 1b, Method). After adjusting for multiple tests using Bonferroni correction, we discovered more than 6,000 CpG sites with significant causal effects on each trait (FIG. 1c). We then performed a pairwise conditional and colocalization (PWCoCo, Method), which is an important method to control false positive results from MR and filter out the MR signals purely driven by LD or pleiotropy⁷⁰. We used the conditional H4 threshold of 0.7 to identify colocalized signals and detected such signals for more than half of the CpG sites identified by MR for each trait (FIG. 1b).

Since we could only perform MR and colocalization analysis on 420,509 CpG sites, the role of unmeasured CpG sites on a tested trait could not be differentiated from the measured ones. To further validate whether the effect estimated by MR can be attributed to a single CpG site, we utilized the point mutation that naturally occurs on the putative causal CpG sites (C to A or C to T), also known as meSNP. For the human methylation array, nearly 10% of CpG sites have an meSNP available. We found that the meSNPs were significantly depleted at putative causal CpG sites, suggesting that there may be a negative selection against loss-of-function mutations at these sites, possibly due to the enrichment of causal sites in regulatory regions (FIGS. 8A-B). Among putative causal CpG sites with meSNPs available, we examined the correlation between the effects on the outcome trait estimated using a single meSNP and the effect estimated by MR. We observed a significant positive correlation between the two estimates (P=1e-4, Pearson's R=0.4, FIGS. 8A-B). These results suggest that the causal effect estimated by MR can be partially attributed to a single CpG site, at least in the putative causal CpG sites with available meSNPs. Yet, considering many CpG sites do not have meSNPs available and the methylation level of individual CpG site tends to be highly correlated with neighboring CpG sites^{48,71,72}, we believe the putative causal CpG sites we identified also serve as tagging CpG sites for the causal regulatory region, and the causal effect size we estimated can be interpreted as the causal effect size of the tagged regulatory region.

Interestingly, the Spearman correlation of the estimated effect size of CpGs across twelve traits formed two distinct clusters, with the first cluster containing eight lifespan- and health-span-related traits, and the second all four epigenetic age measurements (FIG. 1d). This observation suggests that, although all these twelve traits are genetically correlated with each other, causal CpGs do not have proportional effect sizes—the CpGs with large effects on lifespan and healthspan do not have a proportional effect size on epigenetic age measurements and vice versa.

To prioritize CpG sites with a potential causal effect on Aging-GIP1, we first filtered MR signals based on the P value threshold after Bonferroni correction. The CpG sites were then ranked according to the magnitude of the causal effect, adjusted by the colocalization probability (PP.H4). The top CpG sites whose methylation was observed to promote healthy longevity (Aging-GIP1) included cg12122041 at the HTT locus, which is associated with bone mineral density and age, cg02613937 at the TOMM40 locus, which is associated with Alzheimer's disease and age, and cg19047158 at the non-coding region, which is associated with gestational age and rheumatoid arthritis. The top CpG sites whose methylation was found to inhibit healthy longevity included cg04977528 at the HEYL locus, which is associated with sex and age, cg06286026 at the GRK4 locus (associated with age), cg27161488 at the C4orf10 locus (associated with rheumatoid arthritis and age), and cg18744360 at the MAD1L1 locus (associated with hypotensive disorder, FIG. 1e). Furthermore, cg19514613 at the APOE locus is also among the top sites that limit longevity. Genetic variants near HTT and MAML3 were also shown to significantly affect lifespan in Finnish and Japanese cohorts in a previous study⁷³. Both TOMM40 and APOE are known to contribute to the risk of Alzheimer's disease and are associated with human lifespan^{74,75}. Our results suggest that the known lifespan-related effect at these loci may be mediated by DNA methylation. Moreover, we also used adjusted Aging-GIP1, where the effects on human lifespan and healthspan that are correlated with socioeconomic status are removed. We showed that after adjusting for socioeconomic status, the CpG site with the top pro-longevity effect is cg06636172 at the FOXO locus, which is a major longevity locus^{76,77}.

### Example 2. Putative Causal CpG Sites are Enriched in Regulatory Regions

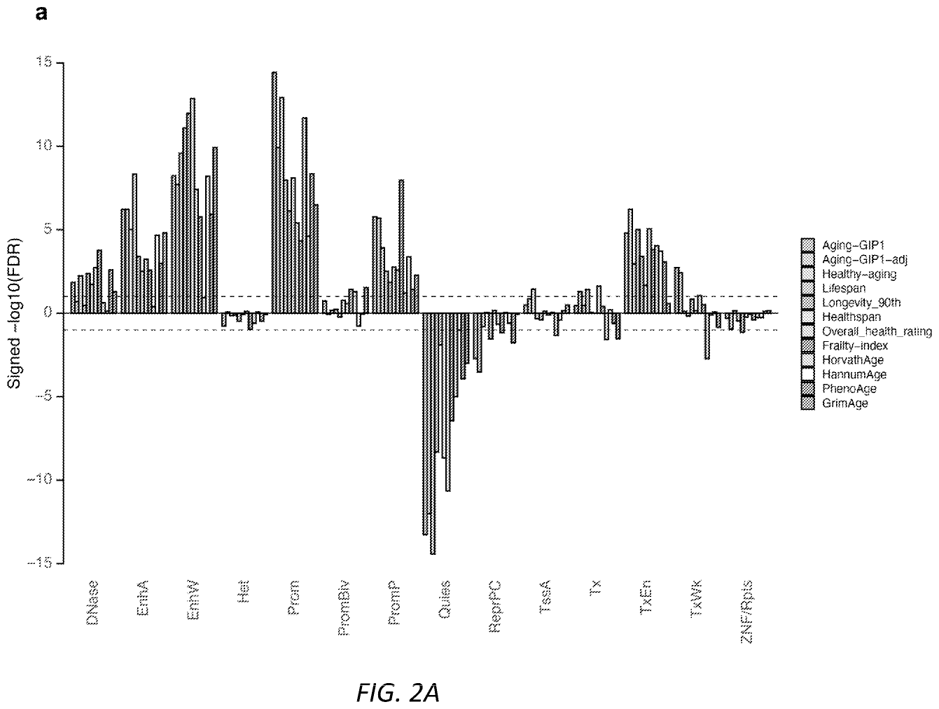

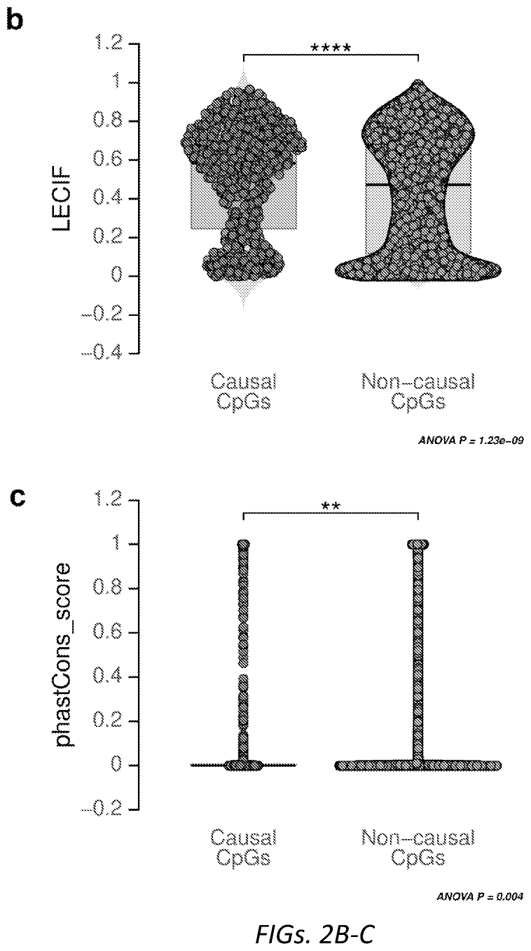

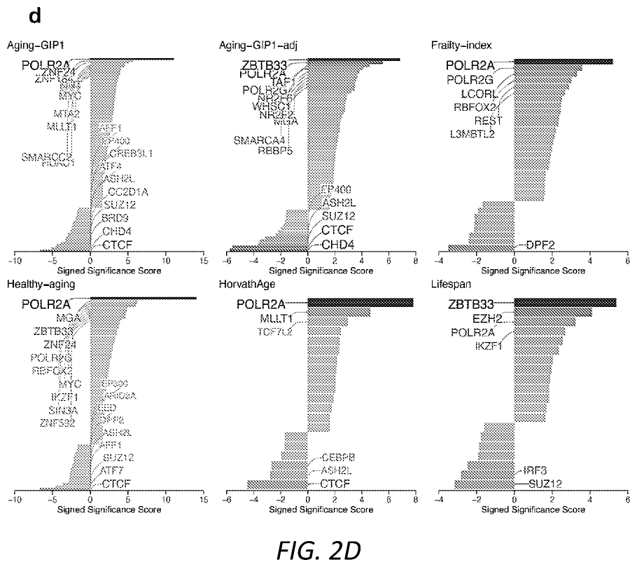

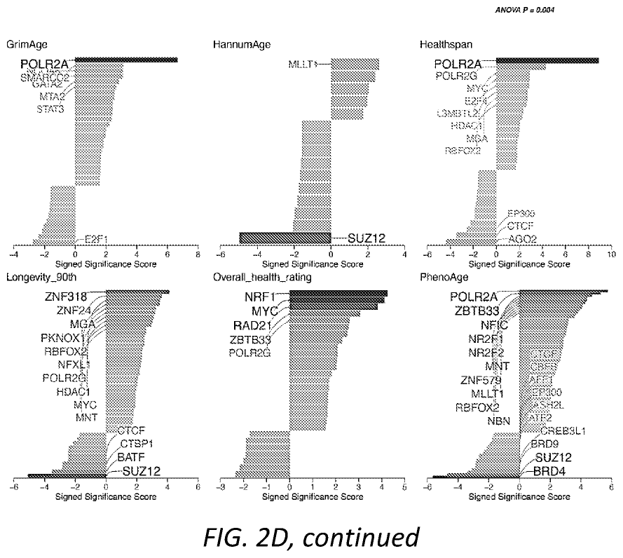

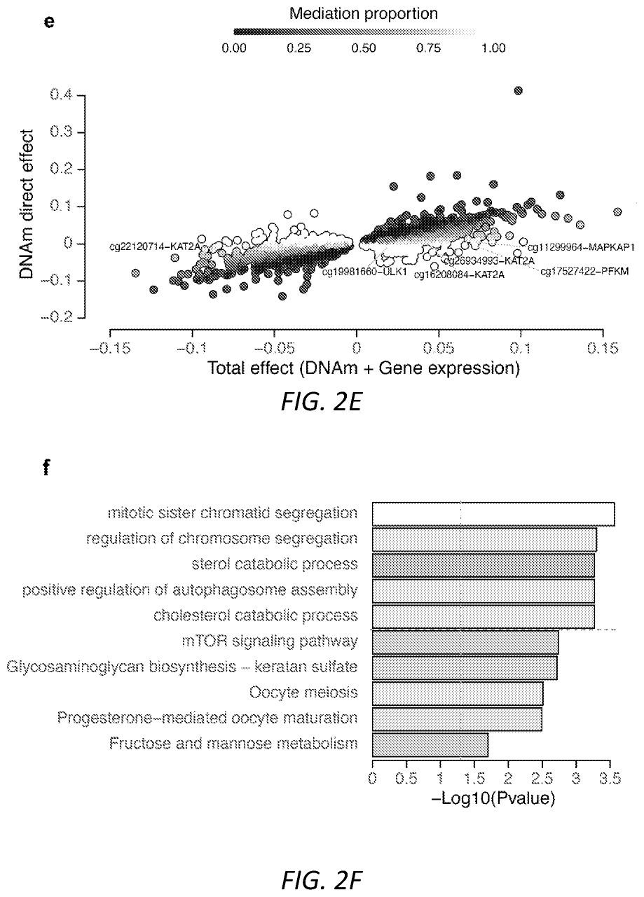

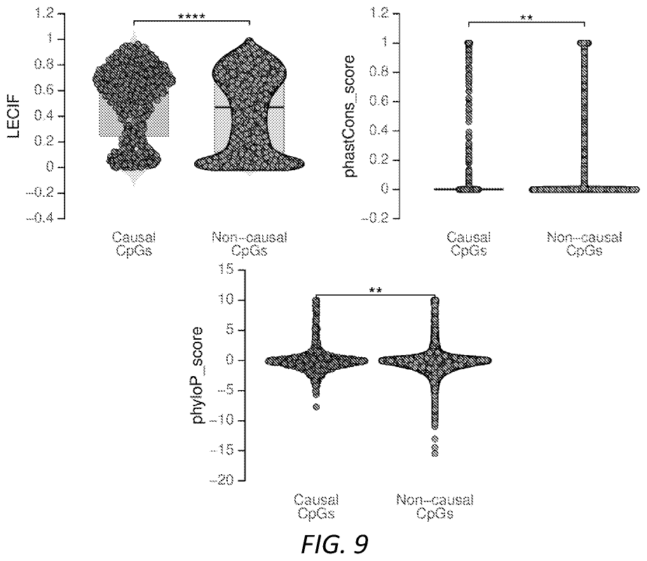

To further understand the properties of the CpG sites identified as causal to each aging-related trait, we performed an enrichment analysis using 14 Roadmap annotations⁷⁸. We found that the putative causal CpGs for most traits are enriched in promoters and enhancers while depleted in quiescent regions (FIG. 2a). Furthermore, these sites were enriched in CpG shores (FIG. 9). We observed that the putative causal CpG sites for Aging-GIP1 are significantly more evolutionally conserved compared to non-causal CpGs, based on both functional genomic conservation scores (Learning Evidence of Conservation from Integrated Functional genomic annotations, LECIF) and the phastCons/phyloP scores across 100 vertebrate genomes⁷⁹ (FIG. 2b, c, FIG. 10). Moreover, the absolute value of the estimated causal effect sizes showed significant positive correlations between all three conservative scores. These results suggest that the CpG sites identified as causal for aging-related traits are more likely to be located in functional genomic elements and more evolutionarily conserved.

It is well known that DNA methylation status may affect the binding of transcription factors (TFs)⁸⁰. To understand the relationship between putative causal CpG sites and TFs, we performed a transcription factor binding site enrichment analysis (FIG. 2d). The CpG sites causal to Aging-GIP1 were significantly enriched in the binding sites of 63 TFs, including POLR2A, ZNF24, MYC, and HDAC1; while depleted in the binding sites of 19 TFs, including CTCF, CHD4, and BRD9 (FIG. 2d). In particular, POLR2A was among the top enriched TFs in 9 of 12 traits. POLR2A is the POLR2 subunit (RNA polymerase II), and previous research shows that epigenetic modifications can modulate its elongation and affect alternative splicing. Our results imply that this mechanism is potentially a major contributor that mediates the effects of DNA methylation on aging^{11,12,81}. We further found that there were 3 TF-binding sites (BRD4, CREB1, and F2F1) enriched with CpG sites whose methylation levels promote healthy longevity (Aging-GIP1), and 4 TF-binding sites (HDAC1, ZHX1, IKZF2, and IRF1) enriched with CpG sites whose methylation levels decrease healthy longevity. BRD4 contributes to cellular senescence and promotes inflammation³⁴. Therefore, our findings suggest that higher DNA methylation at BRD4 binding sites may inhibit the downstream effects of BRD4 and promote healthy longevity. Similarly, previous studies showed that CREB1 is related to type II diabetes and neurodegeneration³⁵, and mediates the effect of calorie restriction³⁶. However, how DNA methylation may affect CREB1 binding is not well studied. Our data suggest that higher methylation at CREB1-binding sites may promote its longevity effects. HDAC1 is a histone deacetylase, and its activity increases with aging and may promote age-related phenotypes^{30,37}. HDAC1 has been shown to specifically bind to methylated sites. Our data, therefore, support the hypothesis that HDAC1 plays a damaging role during aging, as increased DNA methylation at HDAC1 binding sites may causally inhibit healthy longevity.

Since the putative CpGs are enriched in regulatory regions and TF binding sites, we further performed a mediation analysis to investigate whether the effect top CpG hits are mediated through gene expression. The mediation effects were estimated through multivariable MR including both DNA methylation and gene expression, which dissect significant CpG-phenotype causal effects (θ_{T}) into direct effects (θ_{D}) and indirect effects through transcript levels (Method)⁶⁵. Among 2,255 putative causal CpGs applicable to mediation analysis for Aging-GIP1, we found 1,000 of them have their effect mediated by a major transcript (with mediation proportion >0.03, FIG. 2e). For example, we found that the 92% of the effect of cg11299964 on Aging-GIP1 is mediated through the expression of MAPKAP1, which is a key protein in the mTOR signalling pathway (FIG. 2e)⁸²; 83% of the effect of cg22120714 is mediated through the expression of KAT2A, a repressor of NF-kappa-B⁸³. We then performed a gene set enrichment analysis on GO and KEGG using the mediator genes for Aging-GIP1 (FIG. 2f). We found that the mediators are enriched in several aging-related pathway, including mTOR signalling (P=0.0018) and autophagosome assembly (P=5.4e-4, FIG. 2f).

We also examined the enrichment of putative causal CpG sites in phenome-wide EWAS signals obtained from the EWAS catalog 12. The top enriched phenotypes included rheumatoid arthritis, HIV infection, nitrogen dioxide exposure, and maternal obesity. Interestingly, none of these conditions is primarily caused by aging. On the contrary, both rheumatoid arthritis and HIV infection are the conditions that have been suggested to accelerate aging and immunosenescence⁸¹. Additionally, maternal obesity is associated with accelerated metabolic aging in offspring⁸⁴, and nitrogen dioxide exposure is also shown to be associated with an increased risk of mortality⁸⁵. Among the 12 traits tested, only the putative causal CpG sites for GrimAge and Hannum age (both are epigenetic biomarker traits) were significantly enriched in the change of the CpG sites with aging, both epigenetic biomarker traits (FIG. 2e). Therefore, our results suggest that the causal CpG sites for aging are enriched in conditions that cause accelerated aging, but not in conditions that are caused by aging. This is consistent with the previous study, which suggests that differentially expressed genes reflect disease-induced rather than disease-causing changes 86.

### Example 3. MR on Epigenetic Age Measurements Successfully Recovers Clock Sites as Putative Causal CpG Sites

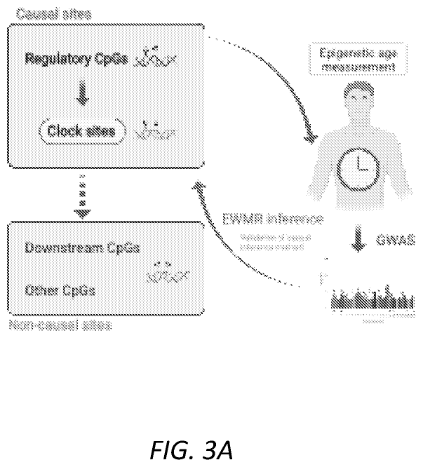

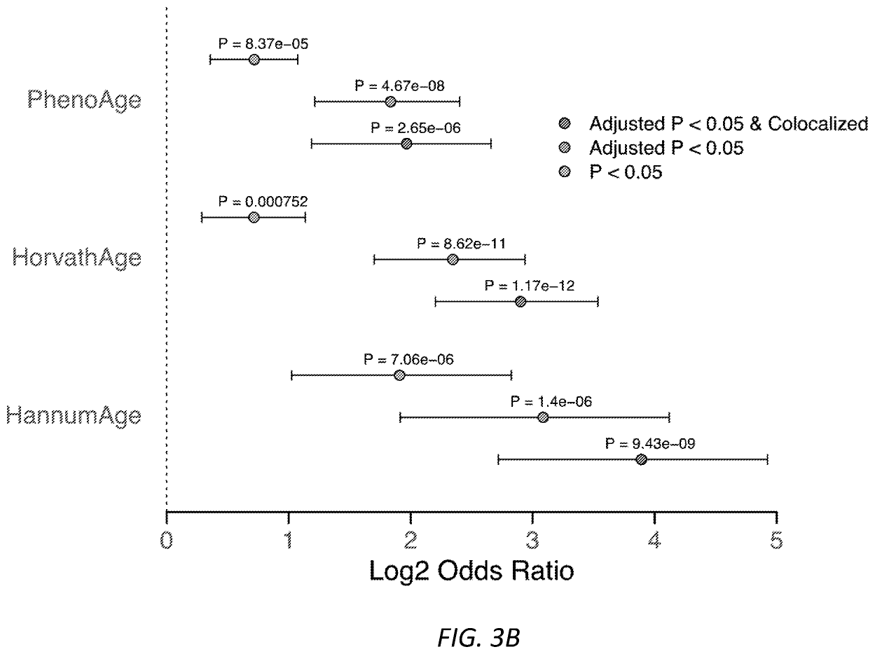

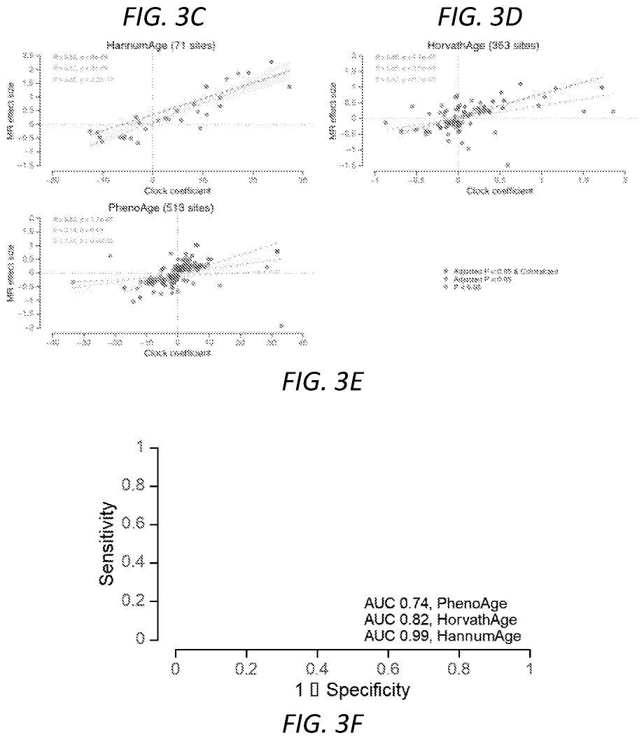

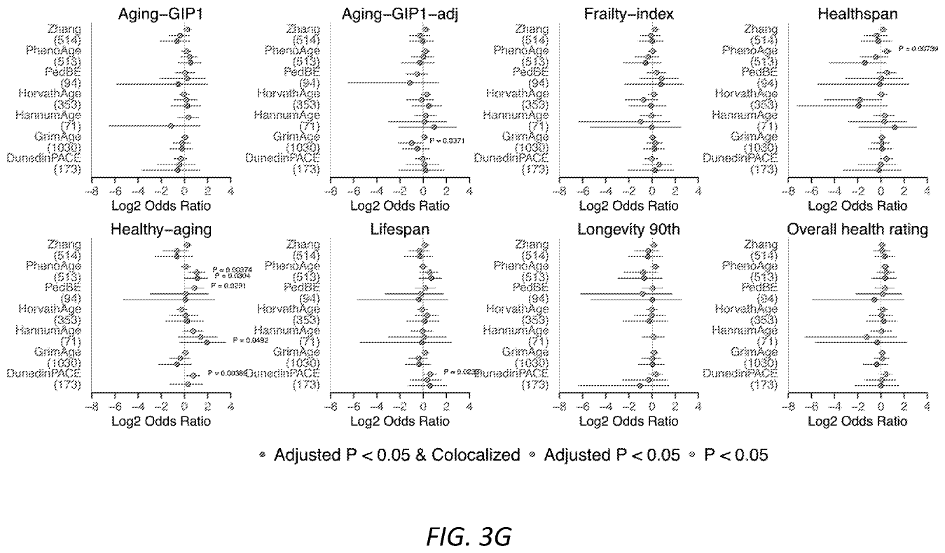

For epigenetic age measurements, the causal CpG sites were the clock sites and the sites upstream of clock sites (FIG. 3a). To validate our EWMR approach for discovering putative causal CpG sites, we used clock sites for each clock as ground truth and investigated whether MR, when using the clock trait as outcome, could recover the clock sites as putative causal CpG sites with the correct estimated effects.

We first examined the identified putative causal CpG sites for three epigenetic age measurements with the clock models publicly available, namely HannumAge, HorvathAge, and PhenoAge⁸. We observed that the CpGs identified by EWMR for each epigenetic age measurement were significantly enriched with the corresponding clock sites (FIG. 3b; HannumAge P=9.4e-9, HorvathAge P=1.2e-12, PhenoAge P=2.7e-6). Furthermore, EWMR predicted causal effect sizes of putative causal CpGs with the correct direction and relative magnitude; as for the three epigenetic age measurements, the estimated causal effect of MR showed a high and significant linear relationship with the actual causal effect sizes denoted by the coefficients of the clock model (FIGS. 3c-e). Notably, the enrichment and correlation we described were also robust to the choice of threshold (FIGS. 3b-e).

In MR studies, the P value is not a reliable ranking metric, as it is largely related to the number of instruments available for the exposure traits²⁷. As the epigenetic age GWAS provided a unique opportunity where a part of the real causal CpG sites was already known, we applied four different ranking metrics to identify an ideal ranking metric to rank putative causal CpG sites. We calculated the area under the receiver operating curve (ROC, AUROC) using the clock sites as ground truth. The AUROC measures the accuracy of binary classification, where an AUROC of 0.5 corresponds to a random classification, and an AUROC of 1 corresponds to a perfect classification. Note that since some putative causal CpGs are unknown (regulatory CpGs upstream to clock sites, FIG. 3a), the AUROC we calculated underestimated the real accuracy. However, we found that when ranking with PP-H4 weighted effect size, strikingly higher AUROCs were achieved compared to all other ranking metrics (0.99 for HannumAge, 0.83 for HorvathAge, and 0.73 for PhenoAge, FIG. 3f). As far as we know, the colocalization probability-weighted effect size has never been used for ranking MR hits. Therefore, our findings provide novel metrics that could be reliably used to prioritize MR results of molecular traits and facilitate downstream analyses.

### Example 4. Existing Epigenetic Clocks are not Enriched with CpG Sites Causal to Aging

One open question for epigenetic clocks is whether their clock sites are causal to aging and age-related functional decline. To answer this question, we collected seven epigenetic age models in humans, namely, the Zhang clock, PhenoAge, GrimAge, PedBE, HorvathAge, HannumAge, and DunedinPACE. We then performed an enrichment analysis of putative causal CpGs for all eight lifespan/healthspan-related traits for each clock. After correcting for multiple testing, none of the existing clocks showed significant enrichment for putative causal CpGs of any of the lifespan/healthspan-related traits (FIG. 3g). PhenoAge showed a nominal significant enrichment with CpGs causal to healthspan and healthy aging, but it was not robust to the choice of thresholds. This finding suggests that, although some clocks contain CpGs causal to aging (Table 2), they, by design, favor CpG sites with a higher correlation with age and thus are not enriched with putative causal CpGs.

In contrast, even though different clocks were trained on different datasets with different methods, the causal sites identified for one clock were usually also enriched with the clock sites for other clocks, suggesting that there is a subset of CpG sites that contribute to the epigenetic age estimate of all existing epigenetic clocks, which could potentially introduce systemic bias.

### Example 5. Integration of MR Results and Age-Associated Differential Methylation Reveals Protective and Deleterious Epigenetic Changes During Aging

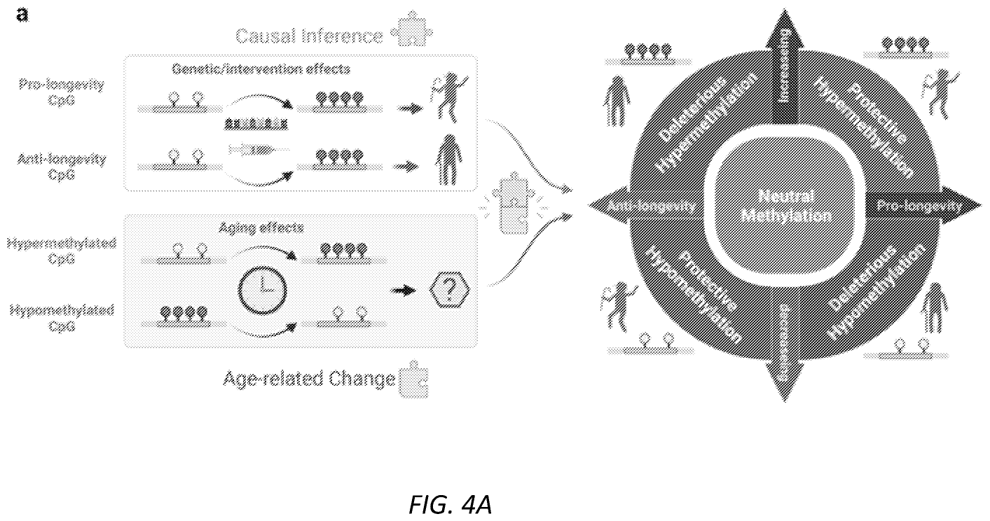

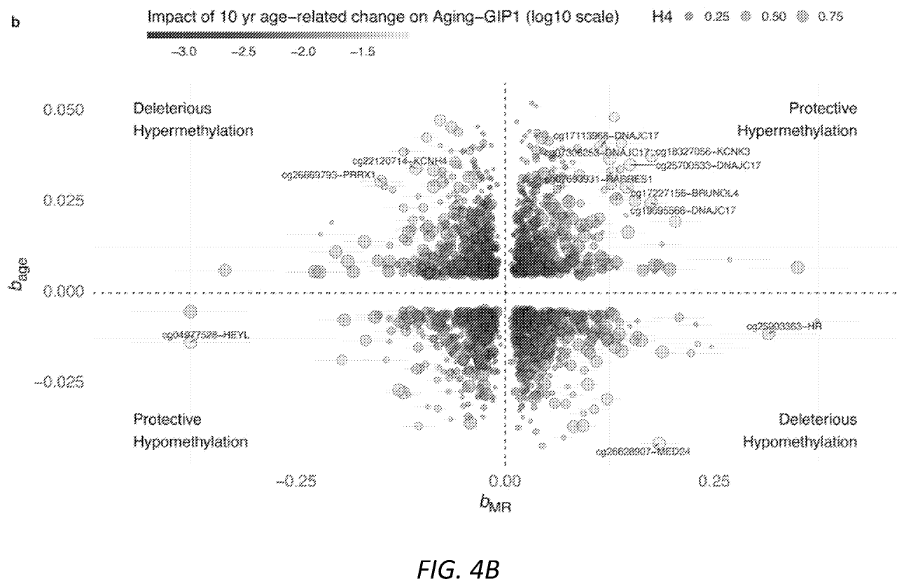

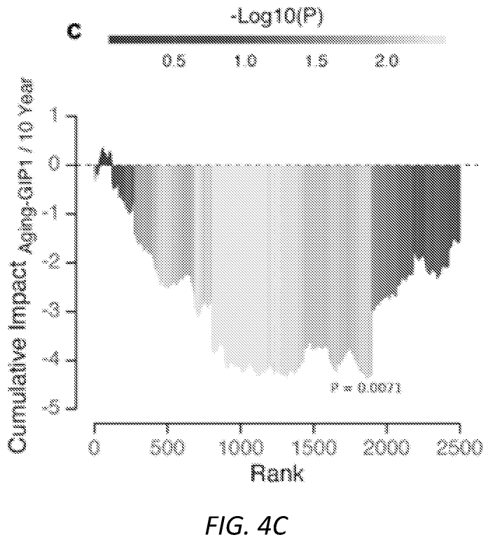

Another important question in epigenetic aging is the identity and number of epigenetic changes that (i) contribute to age-related damage and (ii) respond to it. We approached this question by integrating information on the causal effect and age-related differential methylation for each CpG. The protective or damaging nature of age-related differential methylation at each CpG is indicated by the product of the causal effect and age-associated differential methylation (b_{age}×b_{MR}, FIG. 4a). For example, if a higher methylation level of a certain CpG site leads to a longer lifespan or healthspan, then during aging, a decrease of the methylation level at that site would be considered as having a damaging effect, whereas an increased methylation level would be considered as having a protective effect.

The effect of DNA methylation estimated by MR is estimated through linear regression, which assumes that the relationship between DNA methylation level and lifespan-related outcome is linear. Prior to annotating protective and damaging CpGs, it is important to make sure the effect size of genetic instruments on DNA methylation levels is in the same order of magnitude as the effect of aging. We show that the effect of genetic instruments is comparable with the effect of aging by calculating the ratio between the effect of strongest cis-meQTL and age-related differential methylation for each CpG site. The median ratio was 21.8 for all significant age-associated sites and 3.9 for top 50 age-associated sites, suggesting that the median effect of genetic instruments is roughly equivalent to the effect of years of aging.

Therefore, using the age-related blood DNA methylation data estimated from 7,036 individuals (ages of 18 and 93 years, Generation Scotland cohort)²⁷, we separated the CpG sites causal to eight traits related to lifespan into four different categories: protective hypermethylation, deleterious hypermethylation, protective hypomethylation, and deleterious hypomethylation (FIG. 4b). Among the top 10 CpG sites whose differential methylation during aging has a relatively large impact on healthy longevity, six hypermethylated CpG sites during aging exhibit strong protective effects, including cg18327056, cg25700533, cg19095568, cg17227156, cg17113968, and cg07306253; while one hypomethylated CpG site (cg04977528) also has a protective effect. In contrast, one hypermethylated CpG site (cg26669793) and two hypomethylated CpG sites (cg25903363 and cg26628907) show damaging effects (FIG. 4b).

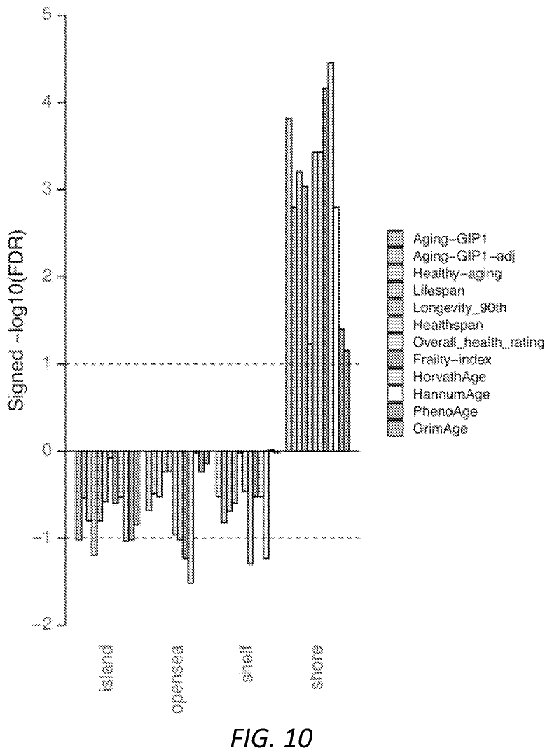

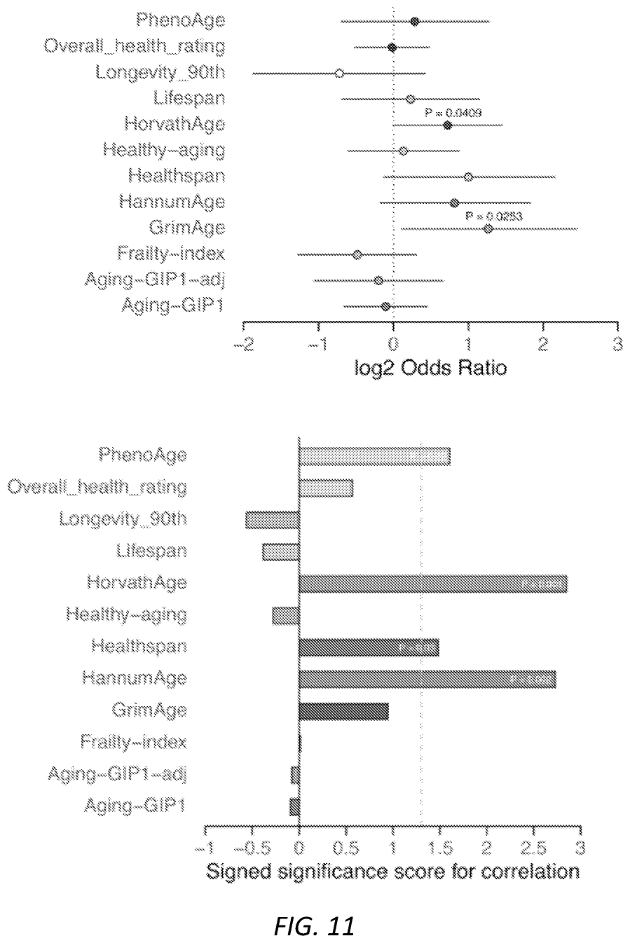

Contradicting the popular notion that most age-related differential methylation features are bad for the organism, our findings revealed that, in terms of the number of CpGs, there was no enrichment for either protective or damaging differential methylation during aging (FIG. 11). Note that the age-associated CpG sites are identified in cross-sectional studies, therefore, a fraction of protective sites we observed could be explained by survival bias (i.e., CpG sites that promote late-life survival). Interestingly, there is a stronger depletion of meSNP in adaptive sites compared to the damaging sites, consistent with the notion that the adaptative mechanism is stronger regulated compared to the damage (FIGS. 8A-B)^{87,88}. We also found that there was no significant correlation between the size of the causal effect and the magnitude of age-associated differential methylations (FIG. 4b, FIG. 11), suggesting that CpG sites with a greater effect on healthy longevity do not necessarily change their level of methylation during aging. This result is consistent with our findings discussed above and explains the lack of enrichment of causal sites in existing epigenetic clocks.

The product of the causal effect and age-associated differential methylation (b_{age}×b_{MR}) provides an estimate of the effect of age-related differential methylation on aging-related phenotypes in a unit of time. We calculated the cumulative effect of age-associated differential methylation on Aging-GIP1 by cumulative summing the effect of top 3,000 age-associated CpG sites, and calculated the empirical P-value through 10,000 permutations (FIG. 4c). Importantly, we discovered that although the number of protective and damaging CpG sites was similar, the cumulative effect of combined age-related DNA differential methylation is significantly detrimental to age-related phenotypes (P=0.007), consistent with the overall damaging nature of aging.

### Example 6. Algorithms for Developing Causality-Informed Epigenetic Clocks

Although various existing epigenetic aging clock models can accurately predict the age of biological samples, they are purely based on correlation. This means that the reliability of existing clock models is highly dependent on the correlation structure of DNA methylation and phenotypes. This may result in unreliable estimates when extrapolating the model to predict the age of novel biological conditions (i.e., applying clocks to interventions that do not exist in the training population), as the correlation structure may be corrupted by the new intervention.

To overcome this problem, we developed novel epigenetic clocks that are based on putative causal CpG sites identified by EWMR (FIG. 5a). Specifically, we trained an elastic net model predicting chronological age on whole blood methylation data from 2,664 individuals^{28,29}, using CpG sites identified as causal to adjusted Aging-GIP1 by EWMR (adjusted P<0.05). In regular epigenetic clock models, the penalty weight is defined to be 1 for all CpG sites, which produces models that are purely based on correlation. Instead, we introduced a novel causality-informed elastic net model, where we assigned the feature-specific penalty factor based on the causality score for each CpG site (Method). The influence of the causality score on the feature-specific penalty factor is controlled by the causality factor τ, which is an adjustable parameter. If τ=0, the whole model is reduced to a regular elastic net regression, where the penalty factor equals one for all features. When τ becomes large, the model is more influenced by the causality score and tends to assign larger coefficients to the features with a higher causality score (FIG. 5A, Methods).

Using this method, we trained the model to build the causality-informed epigenetic clock CausAge (586 sites; see Table A) using 2,664 blood samples. To separately measure adaptive and damaging DNA differential methylation during aging, we further separated putative causal CpG sites into two groups based on the causal effect size from MR and the direction of age-associated differential methylation (FIG. 4b). We then built DamAge, the damaging clock, which contains only the damaging CpG sites (1090 sites; see Table B), and AdaptAge, the protective clock, which contains only the adaptive/protective CpG sites (1000 sites, FIG. 5a; see Table C). We show that the model's accuracy significantly decreased as the causality factor t increased (FIG. 5b, c,). This is because the causality factor t controls the trade-off between the correlation and causality score-weighted penalty factor, and the causality score is not always correlated with the predictive power of age. For example, a CpG site with a high correlation with age may not be causal to aging, and vice versa. We therefore selected causality factor t of 0.3 in the downstream analysis, which is the largest τ value with MAE<5 years in the validation set and maximized the association with mortality (FIGS. 5c-d).

### Example 7. DamAge and AdaptAge Clocks Uncouple Aging-Related Damage and Adaptation

By design, AdaptAge contains only the CpG sites that capture protective effects against aging. Therefore, in theory, the subject predicted to be older by AdaptAge may be expected to accumulate more protective changes during aging. On the contrary, DamAge contains only the CpG sites that exhibit damaging effects, which may be considered as a biomarker of age-related damage. Therefore, we hypothesized that DamAge acceleration may be harmful and shorten life expectancy, whereas AdaptAge acceleration would be protective or neutral, which may indicate healthy longevity.

To test this hypothesis, we first analyzed the associations between human mortality and epigenetic age acceleration quantified by causality-informed clocks using 4,651 individuals from the Framingham Heart Study, FHS offspring cohort (n=2,544 Caucasians, 54% females) and Women's Health Initiative cohort (WHI, n=2107 postmenopausal women, Methods). Among the three causality-informed clocks, DamAge acceleration showed the strongest positive association on mortality (P=9.9e-12) and outperformed CausAge (P=0.01), AdaptAge (P=0.008), Horvath clock (P=0.34), Hannum clock (P=8.2e-7), and PhenoAge (P=9.2e-11, FIG. 5d). This finding supports the notion that age-related damage is the main contributor to the risk of mortality, and the solely damage-base clock is better than the mixture of both damage and adaptation. In contrast, AdaptAge acceleration showed a significant negative association with mortality, suggesting that protective adaptations during aging, measured by AdaptAge, are associated with longer lifespan. In addition, epigenetic age accelerations measured by DamAge and AdaptAge were near-independent (Pearson's R=0.14, FIG. 13). These findings highlight the importance of separating adaptive and damaging age-associated differential methylation when building aging clock models.

Interestingly, although the clock accuracy monotonically decreased as the causality factor t increased, the association between mortality and epigenetic age acceleration did not follow the same trend (FIG. 5d). Especially for DamAge, the mortality association increased as the t increased and peaked when I was around 0.3. Also, DamAge consistently outperformed CausAge in predicting mortality risk, even though CausAge was more accurate in age prediction (FIGS. 5b-e), the association between CausAge and mortality may be weakened due to the inclusion of adaptive sites. This suggests that although the introduction of the causality score and separation of damaging CpGs may decrease the accuracy of the clock in terms of predicting chronological age, it improves the prediction of aging-related phenotypes.

Induced pluripotent stem cell (iPSC) reprogramming is one of the most robust rejuvenation models, which was shown to be able to strongly reverse the epigenetic age of cells^{11,30}. We applied the causality-informed clock models to reprogramming of fibroblasts to iPSC³⁶. For comparison, we also included five published epigenetic models, namely Horvath Age, Hannum Age, PhenoAge, GrimAge and DunedinPACE. The Horvath and Hannum clocks were trained on chronological age^{38,39}, PhenoAge was trained on the age-adjusted by health-related phenotypes^{40,41}, GrimAge was trained on mortality⁸⁹, and DunedinPACE was trained to predict the pace of aging⁴⁰. Consistent with Horvath clock, Hannum clock, PhenoAge, and GrimAge, DamAge revealed that epigenetic age decreased during iPSC reprogramming, but with a stronger negative correlation with the time of reprogramming and higher statistical significance (R=−0.93, P=4e-12, FIG. 5f). This observation suggests that DamAge may better capture the damage-removal effect of iPSC reprogramming. On the contrary, AdaptAge increased significantly during the reprogramming process (R=0.86, P=1.3e-8), suggesting that protective age-associated differential methylation does not capture the rejuvenation effect and that in fact cells may acquire even more protective changes during iPSC reprogramming.

### Example 8. Causality-Informed Epigenetic Clocks Capture Damage and Aging-Related Effects

To further examine how DamAge and AdaptAge capture age-related damage and protective adaptations, respectively, we tested performance of causality-informed clocks using various datasets. For comparison, we included two 1st generation clocks (Horvath age and Hannum age), which are trained solely on chronological age, and three 2nd generation clocks (DunedinPACE, PhenoAge, and GrimAge), which are trained on mortality- and health-related outcomes.

We first examined several aging-related conditions, namely atherosclerosis, cancer, and hypertension (FIG. 6a). We analyzed blood samples from clinical atherosclerosis patients (n=8) and healthy donors (n=8) in the LVAD study⁹⁰. All eight clocks tested showed that the atherosclerosis patients were significantly biologically older than healthy controls (FIG. 6a). We also analyzed 70 prostate cancer cases with good or poor prognosis⁹¹. Only DamAge successfully detected a significant age acceleration in patients with bad cancer prognosis (P=0.039), while Hannum age detected a significant inverse effect where the patients with good prognosis were age accelerated (P=0.044). For hypertensive heart disease, we analyzed blood samples from 44 hypertensive patients and 44 healthy controls⁹². Both CausAge and DamAge showed significant age acceleration in hypertensive patients (CausAge P=0.002, DamAge P=0.04). Similar effects could be detected with GrimAge (P=0.002) and DunedinPACE (P=0.02), but not with AdaptAge, PhenoAge, and two 1st generation clocks. These results suggest that DamAge could more robustly represent the effect of age-related conditions, compared to the published 1st and 2nd generation clocks.

Next, we examined conditions that specifically promote age-related damage (FIG. 6b). Smoking is a well-known risk factor for many age-related diseases, and it also causes DNA damage and oxidative stress⁹³. We compared the epigenetic age of smokers (n=40) and non-smokers (n=40)⁹⁴. CausAge (P=0.004) and DamAge (P=0.006), together with all three 2nd generation clocks could detect significant age-acceleration among smokers, while AdaptAge and two 1st generation clocks did not. Progeroid syndrome is a group of rare genetic disorders that cause premature aging 95. We analyzed blood cell samples from healthy donors (n=3), and patients with Hutchinson-Gilford Progeria Syndrome (HGP, n=3) and Werner Syndrome (n=4)⁹⁶. We observed significant DamAge acceleration in both HGP (P=0.004) and Werner Syndrome (P=5e-4) compared to healthy controls. Similar effects were detected also with PhenoAge and GrimAge. Hannum age and DunedinPACE detected age acceleration in Werner Syndrome but not in HGP, while no significant effect was found by other clocks (FIG. 6b). We then analyzed dermis and epidermis samples with or without sun exposure (n=10 per group) in older adults (age >60)⁹⁷. As the exposure to ultraviolet promotes DNA damage and aging, it may be considered a model of age-related damage. As expected, we observed significant DamAge acceleration in sun-exposed epidermis compared to sun-protected epidermis (P=2e-5), while no significant effect was observed in the dermis tissue. AdaptAge of the sun-exposed epidermis was significantly lower (P=0.01). Surprisingly, based on most other published clocks (including Horvath age, Hannum age, and DunedinPACE), the sun-exposed epidermis was predicted to be significantly younger than sun-protected epidermis. Only GrimAge showed the expected effect direction but did not reach statistical significance (P=0.1).

Paraoxonase 1 (PON1) is one of most studied genes associated with cardiovascular disease, oxidative stress, inflammation, and healthy aging⁴². Specifically, PON1 plays an important role in detoxifying organophosphorus compounds and removing harmful oxidized lipids⁷. The genetic variant of PON1 (R192Q) significantly decreases PON1 activity and is known to be associated with an increased risk of cardiovascular disease and neurodegenerative diseases⁴³. Interestingly, the PON1 Q allele is significantly depleted in centenarians⁴⁴. We analyzed the relationship between PON1 activity and epigenetic age in 48 whole blood samples (FIG. 6a)⁹⁸. DamAge shows a significant negative correlation with PON1 activity (R=−0.55, p=0.0062), whereas AdaptAge showed a significant positive correlation with PON1 activity (R=0.69, p=0.0003). Again, this association was not observed by other epigenetic clocks, except for Horvath age, but with a less significant negative correlation (P=0.04). Thus DamAge can reliably detect damage-related biological age acceleration.

Causality-informed clocks could also capture the aging-related effects of short-term interventions. We first examined the effect of human umbilical cord plasma concentrate injection, which was reported to have age reversal effects⁹⁹. In this study, 18 elderly participants were treated with human umbilical cord plasma concentrate injection weekly (1 ml intramuscular) over a 10-week period. We found that this rejuvenation effect could only be captured with DamAge (P=0.04) and GrimAge (P=0.04), but not with other clocks (FIG. 6c). Similarly, a 6-week omega-3 fatty acid supplementation in overweight subjects (n=34)¹⁰⁰, which was shown to be protective against age-related cardiovascular diseases, significantly increased AdaptAge (P=0.009) and reduced DamAge (P=0.02, FIG. 6c). We also found that short-term treatment with cigarette smoke condensate in bronchial epithelial cells significantly accelerated DamAge (P=0.002) but did not affect other tested clocks (FIG. 6c). Together, our data demonstrate the importance of separating damage and adaptation when building biomarkers of aging and provide novel tools to quantify aging and rejuvenation.

Previous studies have shown that anti-aging interventions delivered during development could prolong lifespan and healthspan, including calorie restriction (CR)¹⁰¹ and rapamycin treatment¹⁰². Small for gestational age (SGA) is a condition defined as birth weight less than the 10th percentile for gestational age¹⁰³. We found that children with SGA have a significantly lower DamAge and higher AdaptAge than children with normal birth weight. These effects were not captured by other epigenetic clocks tested. SGA is usually considered a pathological condition; some studies suggest that it may be because early life benefits can be reversed in later life by exposure to excess nutrients¹⁰⁴. The different roles of SGA in the early and late stages of life may need to be further investigated in future studies.

In vitro fertilization (IVF) is a common method of treating infertility. Yet, previous studies have shown that IVF may increase the risk of perinatal morbidity and mortality¹⁰⁵. We analyzed the DNA methylation data from neonatal blood spots of 137 newborns conceived unassisted (NAT), through intrauterine insemination (IUI), or through IVF using fresh or cryopreserved (frozen) embryo transfer¹⁰⁶. We found that IVF-conceived newborns using fresh or cryopreserved embryos had higher DamAge acceleration and lower AdaptAge than NAT-conceived newborns. On the other hand, IUI-conceived newborns showed no differences in their DamAge and AdaptAge with controls. This effect could not be observed by other five epigenetic clocks tested, except for Horvath age. Genomic imprinting is an epigenetic mechanism that controls the expression of parent-of-origin-dependent gene, which plays an important role in embryonic development and has a lifelong impact on health¹⁰⁷. Some imprinting genes are known to be associated with metabolic disorders and aging (e.g., IGF2-H19)^{108,109}. We analyzed peripheral blood DNA methylation data from patients with single-locus or Multi-loci imprinting disturbances (SLID or MLID), which is the condition of losing methylation at single or multiple imprinting centers¹¹⁰. Similar to IVF, we found that patients with imprinting disorders showed significantly higher DamAge and lower AdaptAge. Together, these results suggest that DamAge and AdaptAge may serve as preferred biomarkers for the events affecting aging traits during development.

TABLE 1
Datasets used in this study

| | |
|---|---|
| Dataset | Description |

GWAS data

| | |
|---|---|
| meQTLs | meQTLs were obtained from the Genetics of DNA Methylation Consortium (GoDMC). DNA |
| | methylation levels were measured in whole blood samples from 36 cohorts, including 27,750 |
| | European subjects. 420,509 CpG sites were analyzed (Min et al.)⁴⁵. |
| Aging-GIP1 | First genetic principal component of six human aging traits—healthspan, father and mother |
| | lifespan, exceptional longevity, frailty index and self-rated health, which captures both length of |
| | life and indices of mental and physical wellbeing (Timmers et al.) ⁵³. |
| Aging-GIP1- | Aging-GIP1 adjusted for household income and socioeconomic deprivation, from the same |
| adj | GWAS study as above ⁵³. |
| Healthy | The multivariate genomic scan of healthspan, lifespan, and longevity (Timmers et al.) 54. |
| aging | |
| Lifespan | GWAS of lifespan from 512,047 mothers and 500,193 fathers of European ancestry (Timmers et al.)⁴⁸. |
| Longevity | 11,262 subjects of European ancestry with a lifespan above the 90th percentile as the case |
| | group and 25,483 control subjects whose age at the last visit was below the 60th percentile age |
| | (Deelen et al.) ⁴⁹. |
| Healthspan | The age of the first incidence of any major age-related disease, including dementia, congestive |
| | heart failure, diabetes, chronic obstructive pulmonary disease, stroke, cancer, myocardial |
| | infarction, as well as the incidence of death. The GWAS of healthspan included 300,447 |
| | subjects of European ancestry from the UK Biobank cohort (Zenin et al.) ⁵⁰. |
| Frailty | Calculated based on the cumulative number of health deficits during aging. The frailty index |
| index | GWAS included 164,610 UK Biobank participants aged 60-70 years and 10,616 Swedish |
| | TwinGene participants aged 41-87 years (Atkins et al.) ⁵⁵. |
| Self-rated | Self-rated health GWAS was based on questionnaire responses on a scale of 0-5 in UK Biobank |
| health | cohort, downloaded from Pan-UKBB project. |
| Horvath age | 1st generation multi-tissue clock trained on chronological age, GWAS was performed on 34,710 |
| | European ancestry and 6,195 African American individuals (McCartney et al) ⁵² |
| Hannum | 1st generation blood clock trained on chronological age, from the same GWAS study as above ⁵². |
| age | |
| PhenoAge | 2st generation blood clock trained on phenotypic age, from the same GWAS study as above ⁵². |
| GrimAge | 2st generation blood clock trained on mortality risk, from the same GWAS study as above ⁵². |

GEO data

| | |
|---|---|
| GSE107143 | This study conducted DNA methylation analyses of blood samples from atherosclerosis patients |
| | and healthy donors. |
| GSE127985 | DNA methylation changes in prostate cancer cases and it's prognosis. |
| GSE192918 | This study analyzed peripheral whole blood DNA methylation profiles of pregnant women at |
| | different stages of gestation and post-delivery, identifying changes in DNA methylation patterns |
| | associated with different time points during pregnancy. |
| GSE193795 | Genome-wide DNA methylation profiling was performed on 44 hypertensive and 44 healthy |
| | control samples, revealing distinct DNA methylation patterns associated with hypertension. |
| GSE210245 | DNA methylation data from human whole blood samples were analyzed to assess the impact of |
| | treatment with human umbilical cord plasma concentrate injection. |
| GSE51954 | Genome-wide DNA methylation profiling was conducted on epidermal and dermal samples |
| | obtained from sun-exposed and sun-protected body sites. |
| GSE94876 | This study compared global methylation changes in buccal cells between smokers, moist snuff |
| | consumers, and non-tobacco consumers. |
| GSE98056 | This study aimed to explore genome-wide DNA methylation changes and identify altered |
| | biological pathways resulting from n-3 fatty acid supplementation in overweight and obese |
| | individuals. |
| GSE101673 | DNA methylation data for cigarette smoke condensate treated cell. |
| GSE78773 | This study identified multi-locus methylation disturbances in individuals with different methylation |
| | patterns, including patients with Temple and Angelman syndromes |
| GSE90117 | This study identified the relationship between PON1 activity, allele, and DNA methylation. |
| GSE79257 | This study analyzed DNA methylation in infants born through different assisted reproductive |
| | techniques and unassisted conception, utilizing archived Guthrie cards for methylation profiling. |
| GSE42865 | This study analyzed DNA methylation in B cells from patients with Hutchinson-Gilford Progeria |
| | Syndrome (HGP) and Werner Syndrome and controls. |

TABLE 2
Putative causal CpG sites in existing epigenetic clocks

| | Position | Weight | outcome | Beta | SE | P | H4 | role |
|---|---|---|---|---|---|---|---|---|
| Horvath | cg06557358 | −0.14 | Overall_health_rating | −0.04 | 0.008 | 1.96E−07 | 0.89 | P |
| Age | cg09509673 | 0.01 | Healthy-aging | 0.02 | 0.003 | 3.86E−13 | 0.85 | P |
| (353) | cg09509673 | 0.01 | Lifespan | 0.05 | 0.006 | 9.92E−20 | 0.83 | P |
| | cg11299964 | −0.16 | Aging-GIP1 | 0.08 | 0.012 | 5.42E−12 | 0.86 | D |
| | cg16744741 | −0.35 | Aging-GIP1 | 0.09 | 0.017 | 1.86E−08 | 0.89 | D |
| | cg16744741 | −0.35 | Overall_health_rating | 0.06 | 0.008 | 6.10E−14 | 0.86 | D |
| Pheno | cg05087948 | −6.99 | Aging-GIP1-adj | −0.08 | 0.013 | 7.30E−10 | 1.00 | P |
| Age | cg21926612 | −2.15 | Overall_health_rating | 0.01 | 0.002 | 3.27E−11 | 0.94 | D |
| (513) | cg11896923 | −1.38 | Healthspan | 0.17 | 0.024 | 4.64E−12 | 0.90 | D |
| | cg11896923 | −1.38 | Healthy-aging | 0.05 | 0.008 | 5.63E−10 | 0.86 | D |
| | cg00862290 | −0.23 | Healthy-aging | 0.00 | 0.001 | 1.28E−08 | 0.85 | P |
| | cg00862290 | −0.23 | Lifespan | −0.02 | 0.003 | 0 | 0.94 | P |
| Zhang | cg24987259 | −1.33 | Overall_health_rating | −0.04 | 0.007 | 8.25E−09 | 0.95 | P |
| (514) | cg05310309 | 0.18 | Aging-GIP1 | 0.03 | 0.003 | 1.13E−32 | 0.96 | P |
| | cg05310309 | 0.18 | Overall_health_rating | 0.01 | 0.002 | 2.64E−12 | 0.92 | P |
| | cg06672696 | 0.02 | Frailty-index | 0.05 | 0.010 | 1.74E−07 | 0.82 | P |
| PedBE | cg04221461 | 0.03 | Frailty-index | 0.04 | 0.008 | 1.25E−07 | 0.95 | P |
| (94) | cg19381811 | −0.08 | Aging-GIP1 | −0.04 | 0.004 | 3.26E−21 | 0.929544 | P |
| | cg19381811 | −0.08 | Overall_health_rating | −0.03 | 0.002 | 8.80E−37 | 0.955032 | P |

P, Protective;
D, deleterious

### REFERENCES

1. Chauhan, A. et al. Systems biology approaches in aging research. Interdiscip Top Gerontol 40, 155-76 (2015).
2. Sen, P., Shah, P. P., Nativio, R. & Berger, S. L. Epigenetic Mechanisms of Longevity and Aging. Cell 166, 822-839 (2016).
3. Guibert, S. & Weber, M. Functions of DNA methylation and hydroxymethylation in mammalian development. Curr Top Dev Biol 104, 47-83 (2013).
4. Jones, P. A. Functions of DNA methylation: islands, start sites, gene bodies and beyond. Nat. Rev. Genet. 13, 484-492 (2012).
5. Moqri, M. et al. PRC2 clock: a universal epigenetic biomarker of aging and rejuvenation. 2022.06.03.494609 Preprint at https://doi.org/10.1101/2022.06.03.494609 (2022).
6. Kane, A. E. & Sinclair, D. A. Epigenetic changes during aging and their reprogramming potential. Crit Rev Biochem Mol Biol 54, 61-83 (2019).
7. López-Otín, C., Blasco, M. A., Partridge, L., Serrano, M. & Kroemer, G. The Hallmarks of Aging. Cell 153, 1194-1217 (2013).
8. Bell, C. G. et al. DNA methylation aging clocks: challenges and recommendations. Genome Biol 20, 249 (2019).
9. A. Reynolds, C. et al. A decade of epigenetic change in aging twins: Genetic and environmental contributions to longitudinal DNA methylation. Aging Cell 19, (2020).
10. Field, A. E. et al. DNA Methylation Clocks in Aging: Categories, Causes, and Consequences. Mol Cell 71, 882-895 (2018).
11. Horvath, S. DNA methylation age of human tissues and cell types. Genome Biol. 14, R115 (2013).
12. Hannum, G. et al. Genome-wide methylation profiles reveal quantitative views of human aging rates. Mol. Cell 49, 359-367 (2013).
13. Chen, B. H. et al. DNA methylation-based measures of biological age: meta-analysis predicting time to death. Aging 8, 1844-1865 (2016).
14. Marioni, R. E. et al. The epigenetic clock is correlated with physical and cognitive fitness in the Lothian Birth Cohort 1936. Int. J. Epidemiol. 44, 1388-1396 (2015).
15. Quach, A. et al. Epigenetic clock analysis of diet, exercise, education, and lifestyle factors. Aging 9, 419-446 (2017).
16. Rutledge, J., Oh, H. & Wyss-Coray, T. Measuring biological age using omics data. Nat. Rev. Genet. (2022) doi: 10.1038/s41576-022-00511-7.
17. Kapur, K. Chapter 14-Principles of Biostatistics. in Clinical and Translational Science (Second Edition) (eds. Robertson, D. & Williams, G. H.) 243-260 (Academic Press, 2017). doi: 10.1016/B978-0-12-802101-9.00014-4.
18. Davey Smith, G. & Hemani, G. Mendelian randomization: genetic anchors for causal inference in epidemiological studies. Hum Mol Genet 23, R89-98 (2014).
19. Evans, D. M. & Davey Smith, G. Mendelian Randomization: New Applications in the Coming Age of Hypothesis-Free Causality. Annu Rev Genomics Hum Genet 16, 327-50 (2015).
20. Neumeyer, S., Hemani, G. & Zeggini, E. Strengthening Causal Inference for Complex Disease Using Molecular Quantitative Trait Loci. Trends Mol Med 26, 232-241 (2020).
21. Võsa, U. et al. Large-scale cis- and trans-eQTL analyses identify thousands of genetic loci and polygenic scores that regulate blood gene expression. Nat. Genet. 53, 1300-1310 (2021).
22. Garrido-Martin, D., Borsari, B., Calvo, M., Reverter, F. & Guigó, R. Identification and analysis of splicing quantitative trait loci across multiple tissues in the human genome. Nat. Commun. 12, 727 (2021).
23. He, B., Shi, J., Wang, X., Jiang, H. & Zhu, H.-J. Genome-wide pQTL analysis of protein expression regulatory networks in the human liver. BMC Biol. 18, 97 (2020).
24. Kraus, W. E. et al. Metabolomic Quantitative Trait Loci (mQTL) Mapping Implicates the Ubiquitin Proteasome System in Cardiovascular Disease Pathogenesis. PLOS Genet 11, e1005553 (2015).
25. Huan, T. et al. Genome-wide identification of DNA methylation QTLs in whole blood highlights pathways for cardiovascular disease. Nat Commun 10, 4267 (2019).
26. Richardson, T. G. et al. Systematic Mendelian randomization framework elucidates hundreds of CpG sites which may mediate the influence of genetic variants on disease. Hum. Mol. Genet. 27, 3293-3304 (2018).
27. McCartney, D. L. et al. An epigenome-wide association study of sex-specific chronological ageing. Genome Med. 12, 1 (2019).
28. Huh, I., Zeng, J., Park, T. & Yi, S. V. DNA methylation and transcriptional noise. Epigenetics Chromatin 6, 9 (2013).
29. Kim, S., Park, H. J., Cui, X. & Zhi, D. Collective effects of long-range DNA methylations predict gene expressions and estimate phenotypes in cancer. Sci. Rep. 10, 1-12 (2020).
30. Lu, Y. et al. Reprogramming to recover youthful epigenetic information and restore vision. Nature 588, 124-129 (2020).
31. Porcu, E. et al. Differentially expressed genes reflect disease-induced rather than disease-causing changes in the transcriptome. Nat. Commun. 12, 5647 (2021).
32. Emdin, C. A., Khera, A. V. & Kathiresan, S. Mendelian Randomization. JAMA 318, 1925-1926 (2017).
33. Lin, D. et al. Characterization of cross-tissue genetic-epigenetic effects and their patterns in schizophrenia. Genome Med. 10, 13 (2018).
34. Burgess, S., Zuber, V., Valdes-Marquez, E., Sun, B. B. & Hopewell, J. C. Mendelian randomization with fine-mapped genetic data: Choosing from large numbers of correlated instrumental variables. Genet. Epidemiol. 41, 714-725 (2017).
35. Lehne, B. et al. A coherent approach for analysis of the Illumina HumanMethylation450 BeadChip improves data quality and performance in epigenome-wide association studies. Genome Biol. 16, 37 (2015).
36. Ohnuki, M. et al. Dynamic regulation of human endogenous retroviruses mediates factor-induced reprogramming and differentiation potential. Proc. Natl. Acad. Sci. U.S.A 111, 12426-12431 (2014).
37. Rando, T. A. & Chang, H. Y. Aging, Rejuvenation, and Epigenetic Reprogramming: Resetting the Aging Clock. Cell 148, 46-57 (2012).
38. Tollefsbol, T. O. Techniques for Analysis of Biological Aging. Methods Mol. Biol. Clifton NJ 371, 1-7 (2007).
39. Tosato, M., Zamboni, V., Ferrini, A. & Cesari, M. The aging process and potential interventions to extend life expectancy. Clin. Interv. Aging 2, 401-412 (2007).
40. Fontana, L., Partridge, L. & Longo, V. D. Extending healthy life span—from yeast to humans. Science 328, 321-6 (2010).
41. Milman, S. et al. Low insulin-like growth factor-1 level predicts survival in humans with exceptional longevity. Aging Cell 13, 769-771 (2014).
42. Schumacher, B. et al. Delayed and Accelerated Aging Share Common Longevity Assurance Mechanisms. PLOS Genet. 4, e1000161 (2008).
43. Walther, D. M. et al. Widespread Proteome Remodeling and Aggregation in Aging C. elegans. Cell 161, 919-932 (2015).
44. Goto, K. et al. Protective mechanism against age-associated changes in the peripheral nerves. Life Sci. 253, 117744 (2020).
45. Min, J. L. et al. Genomic and phenotypic insights from an atlas of genetic effects on DNA methylation. Nat. Genet. 53, 1311-1321 (2021).
46. Griffith, G. et al. Collider bias undermines our understanding of COVID-19 disease risk and severity. medRxiv 2020.05.04.20090506 (2020) doi:10.1101/2020.05.04.20090506.
47. Zhou, Z., Rahme, E., Abrahamowicz, M. & Pilote, L. Survival Bias Associated with Time-to-Treatment Initiation in Drug Effectiveness Evaluation: A Comparison of Methods. Am. J. Epidemiol. 162, 1016-1023 (2005).
48. Timmers, P. R. et al. Genomics of 1 million parent lifespans implicates novel pathways and common diseases and distinguishes survival chances. eLife 8, e39856 (2019)
49. Deelen, J. et al. A meta-analysis of genome-wide association studies identifies multiple longevity genes. Nat. Commun. 10, 3669 (2019).
50. Zenin, A. et al. Identification of 12 genetic loci associated with human healthspan. Commun. Biol. 2, 41 (2019).
51. Kojima, G., Iliffe, S. & Walters, K. Frailty index as a predictor of mortality: a systematic review and meta-analysis. Age Ageing 47, 193-200 (2018).
52. McCartney, D. L. et al. Genome-wide association studies identify 137 genetic loci for DNA methylation biomarkers of aging. Genome Biol. 22, 194 (2021).
53. Timmers, P. R. H. J. et al. Mendelian randomization of genetically independent aging phenotypes identifies LPA and VCAM1 as biological targets for human aging. Nat. Aging 2, 19-30 (2022).
54. Timmers, P., Wilson, J. F., Joshi, P. K. & Deelen, J. Multivariate genomic scan implicates novel loci and haem metabolism in human ageing. Nat Commun 11, 3570 (2020).
55. Atkins, J. L. et al. A genome-wide association study of the frailty index highlights brain pathways in ageing. Aging Cell 20, e13459 (2021).
56. Bulik-Sullivan, B. K. et al. L D Score regression distinguishes confounding from polygenicity in genome-wide association studies. Nat Genet 47, 291-5 (2015).
57. Burgess, S., Butterworth, A. & Thompson, S. G. Mendelian randomization analysis with multiple genetic variants using summarized data. Genet. Epidemiol. 37, 658-665 (2013).
58. Zhao, H. et al. Proteome-wide Mendelian randomization in global biobank meta-analysis reveals multi-ancestry drug targets for common diseases. 2022.01.09.21268473 Preprint at https://doi.org/10.1101/2022.01.09.21268473 (2022).
59. Yavorska, O. O. & Burgess, S. MendelianRandomization: an R package for performing Mendelian randomization analyses using summarized data. Int. J. Epidemiol. 46, 1734-1739 (2017).
60. Hemani, G. et al. The MR-Base platform supports systematic causal inference across the human phenome. eLife 7, e34408 (2018).
61. Bowden, J., Davey Smith, G. & Burgess, S. Mendelian randomization with invalid instruments: effect estimation and bias detection through Egger regression. Int J Epidemiol 44, 512-25 (2015).
62. Hemani, G., Tilling, K. & Davey Smith, G. Orienting the causal relationship between imprecisely measured traits using GWAS summary data. PLOS Genet. 13, e1007081 (2017).
63. Wallace, C. Eliciting priors and relaxing the single causal variant assumption in colocalisation analyses. PLOS Genet. 16, e1008720 (2020).
64. Zheng, J. et al. Phenome-wide Mendelian randomization mapping the influence of the plasma proteome on complex diseases. Nat. Genet. 52, 1122-1131 (2020).
65. Sadler, M. C., Auwerx, C., Lepik, K., Porcu, E. & Kutalik, Z. Quantifying the role of transcript levels in mediating DNA methylation effects on complex traits and diseases. Nat. Commun. 13, 7559 (2022).
66. Zou, H. & Hastie, T. Regularization and variable selection via the elastic net. J. R. Stat. Soc. Ser. B Stat. Methodol. 67, 301-320 (2005).
67. Handl, L., Jalali, A., Scherer, M., Eggeling, R. & Pfeifer, N. Weighted elastic net for unsupervised domain adaptation with application to age prediction from DNA methylation data. Bioinformatics 35, i154-i163 (2019).
68. Tay, J. K., Aghaeepour, N., Hastie, T. & Tibshirani, R. Feature-weighted elastic net: using ‘features of features’ for better prediction. ArXiv200601395 Cs Stat (2020).
69. Kundaje, A. et al. Integrative analysis of 111 reference human epigenomes. Nature 518, 317-330 (2015).
70. Robinson, J. W. et al. An efficient and robust tool for colocalisation: Pair-wise Conditional and Colocalisation (PWCoCo). 2022.08.08.503158 Preprint at https://doi.org/10.1101/2022.08.08.503158 (2022).
71. Shao, Y. et al. DNA methylation of TOMM40-APOE-APOC2 in Alzheimer's disease. J. Hum. Genet. 63, 459-471 (2018).
72. Goh, L. K. et al. TOMM40 alterations in Alzheimer's disease over a 2-year follow-up period. J. Alzheimers Dis. JAD 44, 57-61 (2015).
73. Hu, S. et al. DNA methylation presents distinct binding sites for human transcription factors. eLife 2, e00726 (2013).
74. González-Rodríguez, P., Klionsky, D. J. & Joseph, B. Autophagy regulation by RNA alternative splicing and implications in human diseases. Nat. Commun. 13, 2735 (2022).
75. Saldi, T., Cortazar, M. A., Sheridan, R. M. & Bentley, D. L. Coupling of RNA Polymerase II Transcription Elongation with Pre-mRNA Splicing. J. Mol. Biol. 428, 2623-2635 (2016).
76. Hua, T. et al. BRD4 Inhibition Attenuates Inflammatory Pain by Ameliorating NLRP3 Inflammasome-Induced Pyroptosis. Front. Immunol. 13, 837977 (2022).
77. Wang, H. et al. BRD4 contributes to LPS-induced macrophage senescence and promotes progression of atherosclerosis-associated lipid uptake. Aging 12, 9240-9259 (2020).
78. Herzig, S. et al. CREB regulates hepatic gluconeogenesis through the coactivator PGC-1. Nature 413, 179-183 (2001).
79. Fusco, S. et al. A role for neuronal cAMP responsive-element binding (CREB)-1 in brain responses to calorie restriction. Proc. Natl. Acad. Sci. 109, 621-626 (2012).
80. Willis-Martinez, D., Richards, H. W., Timchenko, N. A. & Medrano, E. E. Role of HDAC1 in senescence, aging, and cancer. Exp. Gerontol. 45, 279-285 (2010).
81. Levine, M. E. et al. An epigenetic biomarker of aging for lifespan and healthspan. Aging 10, 573-591 (2018).
82. Yuan, Y. et al. Characterization of Sin 1 Isoforms Reveals an mTOR-Dependent and Independent Function of Sinly. PLOS ONE 10, e0135017 (2015).
83. Mao, X. et al. GCN5 is a required cofactor for a ubiquitin ligase that targets NF-kappaB/RelA. Genes Dev. 23, 849-861 (2009).
84. Belsky, D. W. et al. Quantification of the pace of biological aging in humans through a blood test, the DunedinPoAm DNA methylation algorithm. eLife 9, e54870 (2020).
85. Stewart, E. A. et al. Uterine fibroids. Nat. Rev. Dis. Primer 2, 16043 (2016).
86. George, J. W. et al. Integrated Epigenome, Exome, and Transcriptome Analyses Reveal Molecular Subtypes and Homeotic Transformation in Uterine Fibroids. Cell Rep. 29, 4069-4085.e6 (2019).
87. Aledo, J. C. & Blanco, J. M. Aging is Neither a Failure nor an Achievement of Natural Selection. Curr. Aging Sci. 8, 4-10.
88. Flatt, T. & Partridge, L. Horizons in the evolution of aging. BMC Biol 16, 93 (2018).
89. Lu, A. T. et al. DNA methylation GrimAge strongly predicts lifespan and healthspan. Aging 11, 303-327 (2019).
90. Istas, G. et al. Identification of differentially methylated BRCA1 and CRISP2 DNA regions as blood surrogate markers for cardiovascular disease. Sci. Rep. 7, 5120 (2017).
91. Toth, R. et al. Random forest-based modelling to detect biomarkers for prostate cancer progression. Clin. Epigenetics 11, 148 (2019).
92. Bai, C. et al. Oviductal Glycoprotein 1 Promotes Hypertension by Inducing Vascular Remodeling Through an Interaction With MYH9. Circulation 146, 1367-1382 (2022).
93. Csiszar, A. et al. Oxidative stress and accelerated vascular aging: implications for cigarette smoking. Front. Biosci. Landmark Ed. 14, 3128-3144 (2009).
94. Jessen, W. J., Borgerding, M. F. & Prasad, G. L. Global methylation profiles in buccal cells of long-term smokers and moist snuff consumers. Biomark. Biochem. Indic. Expo. Response Susceptibility Chem. 23, 625-639 (2018).
95. Kudlow, B. A., Kennedy, B. K. & Monnat, R. J. Werner and Hutchinson-Gilford progeria syndromes: mechanistic basis of human progeroid diseases. Nat. Rev. Mol. Cell Biol. 8, 394-404 (2007).
96. Heyn, H., Moran, S. & Esteller, M. Aberrant DNA methylation profiles in the premature aging disorders Hutchinson-Gilford Progeria and Werner syndrome. Epigenetics 8, 28-33 (2013).
97. Vandiver, A. R. et al. Age and sun exposure-related widespread genomic blocks of hypomethylation in nonmalignant skin. Genome Biol. 16, 80 (2015).
98. Declerck, K. et al. Interaction between prenatal pesticide exposure and a common polymorphism in the PON1 gene on DNA methylation in genes associated with cardio-metabolic disease risk—an exploratory study. Clin. Epigenetics 9, 35 (2017).
99. Clement, J. et al. Umbilical cord plasma concentrate has beneficial effects on DNA methylation GrimAge and human clinical biomarkers. Aging Cell 21, e13696 (2022).
100. Tremblay, B. L. et al. Epigenetic changes in blood leukocytes following an omega-3 fatty acid supplementation. Clin. Epigenetics 9, 43 (2017).
101. Sun, L., Sadighi Akha, A. A., Miller, R. A. & Harper, J. M. Life-span extension in mice by preweaning food restriction and by methionine restriction in middle age. J. Gerontol. A. Biol. Sci. Med. Sci. 64, 711-722 (2009).
102. Shindyapina, A. V. et al. Rapamycin treatment during development extends life span and health span of male mice and Daphnia magna. Sci. Adv. 8, eabo5482 (2022).
103. Osuchukwu, O. O. & Reed, D. J. Small for Gestational Age. in StatPearls (StatPearls Publishing, 2022).
104. Ribeiro, A. M., Lima, M. de C., de Lira, P. I. C. & da Silva, G. A. P. Low birth weight and obesity: causal or casual casual association? Rev. Paul. Pediatr. 33, 340-348 (2015).
105. Sabban, H., Zakhari, A., Patenaude, V., Tulandi, T. & Abenhaim, H. A. Obstetrical and perinatal morbidity and mortality among in-vitro fertilization pregnancies: a population-based study. Arch. Gynecol. Obstet. 296, 107-113 (2017).
106. Estill, M. S. et al. Assisted reproductive technology alters deoxyribonucleic acid methylation profiles in bloodspots of newborn infants. Fertil. Steril. 106, 629-639.e10 (2016).
107. Monk, D., Mackay, D. J. G., Eggermann, T., Maher, E. R. & Riccio, A. Genomic imprinting disorders: lessons on how genome, epigenome and environment interact. Nat. Rev. Genet. 20, 235-248 (2019).
108. Ratajczak, M. Z. Igf2-H19, an imprinted tandem gene, is an important regulator of embryonic development, a guardian of proliferation of adult pluripotent stem cells, a regulator of longevity, and a ‘passkey’ to cancerogenesis. Folia Histochem. Cytobiol. 50, 171-179 (2012).
109. Butler, M. G. Genomic imprinting disorders in humans: a mini-review. J. Assist. Reprod. Genet. 26, 477 (2009).
110. Bens, S. et al. Phenotypic spectrum and extent of DNA methylation defects associated with multilocus imprinting disturbances. Epigenomics 8, 801-816 (2016).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
providing a biological test system, optionally a cell, tissue, organ, or organism; and
determining methylation of one or more causal CpG sites identified in Tables A, B, and/or C.

2. The method of claim 1, comprising determining methylation of 5, 10, 20, 30, 40, 50, 60, 70, 80, 90, 100, 125, 150, or more of the causal CpG sites.

3. The method of claim 2, comprising determining methylation of up to 100, 125, 150, 175, 200, 250, 300, 350, 400, 500, or 1,000 CpG sites, including determining methylation of at least 5, 10, 20, 30, 40, 50, 60, 70, 80, 90, 100, 125, 150, or all of the causal CpG sites.

4. The method of claim 1, further comprising applying an intervention to the system, and determining methylation of the one or more causal CpG sites during and/or after an application of an intervention.

5. The method of claim 4, further comprising comparing the methylation of the one or more causal CpG sites to a reference pattern of methylation.

6. The method of claim 5, wherein the reference pattern is a baseline methylation pattern obtained in the same test system before application of an intervention, or a level or range obtained earlier in time in the same test system, or a level or range in a reference system that represents the level or range of methylation in the absence of an intervention.

7. The method of claim 1, comprising determining methylation of a plurality of causal CpG sites, and calculating a score using an algorithm to calculate the cumulative effect on aging or aging-related outcomes, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; or neural networks.

8. The method of claim 7, comprising calculating a predicted age using the determined methylation and applying an algorithm to the levels.

9. The method of claim 8, wherein the algorithm comprises:
$$PredictedAge = intercept + b 1 * CpG 1 + b 2 * CpG 2 + … + bn * CpGn$$
Where b1−bn are the model coefficient ‘estimate’ from Tables A, B, and/or C and CpG1−CpGn are the methylation level of given CpG sites (on a scale of 0-1).

10. The method of claim 1, further comprising identifying an intervention as having a protective effect when changes in methylation are observed that are consistent with protection, and/or identifying an intervention as having a damaging effect when changes in methylation are observed that are consistent with damage.

11. The method of claim 10, further comprising:
selecting an intervention that has been identified as having a protective effect as a candidate intervention;
applying the candidate intervention to an in vivo model of a disorder or condition associated with aging, optionally wherein the model is a non-human test animal or a human subject in a clinical trial; and
determining whether the candidate intervention has a protective effect on the disorder or condition related to aging.

12. A method of predicting an effect of an intervention on aging, the method comprising:
providing a biological test system, optionally a cell, tissue, organ, or organism; and
determining methylation of one or more causal CpG sites identified in Tables A, B, and/or C;
applying an intervention to the system,
determining methylation of the one or more causal CpG sites during and/or after an application of an intervention;
comparing the methylation of the one or more causal CpG sites to a reference pattern of methylation; and
identifying an intervention as having a protective effect on aging when changes in methylation are observed that are consistent with protection, and/or identifying an intervention as having a damaging effect on aging when changes in methylation are observed that are consistent with damage.

13. The method of claim 12, comprising determining methylation of 5, 10, 20, 30, 40, 50, 60, 70, 80, 90, 100, 125, 150, or more of the causal CpG sites.

14. The method of claim 13, comprising determining methylation of up to 100, 125, 150, 175, 200, 250, 300, 350, 400, 500, or 1,000 CpG sites, including determining methylation of at least 5, 10, 20, 30, 40, 50, 60, 70, 80, 90, 100, 125, 150, or all of the causal CpG sites.

15. The method of claim 12, wherein the reference pattern is a baseline methylation pattern obtained in the same test system before application of an intervention, or a level or range obtained earlier in time in the same test system, or a level or range in a reference system that represents the level or range of methylation in the absence of an intervention.

16. The method of claim 12, comprising determining methylation of a plurality of causal CpG sites, and calculating a score using an algorithm to calculate the cumulative effect on aging or age-related outcomes, optionally wherein the algorithm comprises using a manual or software-based modeling algorithm, optionally wherein the algorithm comprises a linear algorithm; principal component analysis (PCA); classification or decision trees; elastic net analysis; linear and polynomial support vector machines (SMV); shrunken centroids; random forest algorithms; support vector machines; or neural networks.

17. The method of claim 16, comprising calculating a predicted age using the determined methylation and applying an algorithm to the levels.

18. The method of claim 17, wherein the algorithm comprises:
$$PredictedAge = intercept + b 1 * CpG 1 + b 2 * CpG 2 + … + bn * CpGn$$
where b1−bn are the model coefficient ‘estimate’ from Tables A, B, and/or C and CpG1−CpGn are the methylation level of given CpG sites (on a scale of 0-1).

19. The method of claim 18, further comprising:
selecting an intervention that has been identified as having a protective effect as a candidate intervention;
applying the candidate intervention to an in vivo model of a disorder or condition associated with aging, optionally wherein the model is a non-human test animal or a human subject in a clinical trial; and
determining whether the candidate intervention has a protective effect on the disorder or condition related to aging.
